(19) United States
(12) Patent Application Publication
(10) Pub. No.: US 2006/0236427 A1
(43) Pub. Date: Oct. 19, 2006

(54) MicroRNAs (miRNAs) for plant growth and development

(75) Inventors: Vincent Lee Chuan Chiang, Cary, NC (US); Shanfa Lu, Raleigh, NC (US); Ying-Hsuan Sun, Raleigh, NC (US); Laigeng Li, Cary, NC (US)

Correspondence Address: JENKINS, WILSON, TAYLOR & HUNT, P. A., 3100 TOWER BLVD, SUITE 1200, DURHAM, NC, 27707, US

(73) Assignee: North Carolina State University, Raleigh, NC (US)

(21) Appl. No.: 11/231,318

(22) Filed: Sep. 20, 2005

Related U.S. Application Data
(60) Provisional application No. 60/611,290, filed on Sep. 20, 2004.

(51) Int. Cl. A01H 1/00; C12N 15/82; C12N 15/87

(52) U.S. Cl. 800/284; 800/294

(57) ABSTRACT

The presently disclosed subject matter provides methods and compositions for modulating gene expression in plants. Also provided are plants and cells comprising the compositions of the presently disclosed subject matter.

### CROSS REFERENCE TO RELATED APPLICATIONS

This application is based on and claims priority to U.S. Provisional Application Ser. No. 60/611,290, filed Sep. 20, 2004, the disclosure of which is herein incorporated by reference in its entirety.

### GRANT STATEMENT

This work was supported by grant DE-FG02-03ER15442 from the United States Department of Energy. Thus, the U.S. government has certain rights in the presently disclosed subject matter.

### TECHNICAL FIELD

The presently disclosed subject matter relates, in general, to methods and compositions for modulating gene expression in a plant. More particularly, the presently disclosed subject matter relates to a method of using a microRNA (miRNA) to modulate the expression level of a gene in a plant, and to compositions comprising miRNAs.

### BACKGROUND

Trees are a major natural resource of the biosphere and have shown outstanding ecological and economic importance. A key physiological process of tree development is the formation of wood, which is composed of a variety of cell types.

Wood is made up of plant cell wall lignins, which occur exclusively in higher plants and represent the second most abundant organic compound on the earth's surface after cellulose, accounting for about 25% of plant biomass. Cell wall lignification involves the deposition of phenolic polymers (lignins) on the extracellular polysaccharide matrix. The polymers arise from the oxidative coupling of three cinnamyl alcohols. The main functions of lignins are to strengthen the plant vascular body, provide mechanical support for stems and leaf blades, and to provide resistance to diseases, insects, cold temperatures, and other biotic and abiotic stresses.

Although lignins play many important roles in vascular plants, their resistance to degradation greatly complicates various agricultural and industrial uses of plants. For example, animals lack the enzymes necessary for degrading the polysaccharides in plant cell walls, and thus must depend on microbial fermentation to break down plant fibers. High lignin concentration and methoxyl content reduce the digestibility of forage crops (for example, alfalfa), with cattle (for example) able to digest only 40-50% of legume fibers and 60-70% of grass fibers. Thus, lignins have been implicated in limiting forage digestibility, possibly by interfering with microbial degradation of fiber polysaccharides. Small decreases in lignin content of plants, however, can have a significant positive impact on forage digestibility.

High lignin content also is problematic in the wood products industries, which is an important component of both the United States' and global economies. Up to thirty-six percent of the dry weight of wood is lignin. During pulp and papermaking, lignin must be separated from cellulose. This process consumes large amounts of energy and imposes a high environmental cost due to the requirement for using chemicals such as chlorine bleach. The availability of wood with reduced lignin content or with a modified lignin that is more amenable to extraction would increase the efficiency of pulp and papermaking processes and would decrease chemical consumption and disposal. Thus, both the digestibility of forage crops and the pulping properties of trees can be adversely affected by high lignin content.

Genetic engineering has great promise for agriculture because it can accelerate traditional breeding programs, cross reproductive barriers, and introduce specific desired traits. Genetic engineering can be particularly advantageous to forestry because traditional methods are hampered by the long generation times of trees. Yet, the manipulation of a plant's genome can have undesirable effects.

Thus, there is a long-felt and continuing need in the art for new methods for identifying genes that specifically regulate important developmental pathways of plants. Also needed are new methods for genetically modifying cultivated vascular plants to manipulate the expression of genes of interest. Such methods would improve the ability of vascular plants to be used in agriculture, in the pulp and paper industry, and in other industries. The presently disclosed subject matter addresses this and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for stably modulating expression of a plant gene. In some embodiments, the method comprises (a) providing a vector encoding a microRNA (miRNA) targeted to the plant gene; and (b) transforming a plant cell with the vector, whereby stable expression of the miRNA in the plant cell is provided. In some embodiments, the method comprises (a) transforming a plurality of plant cells with a vector comprising a nucleic acid sequence encoding a microRNA (miRNA) operatively linked to a promoter and a transcription termination sequence; (b) growing the plant cells under conditions sufficient to select for a plurality of transformed plant cells that have integrated the vector into their genomes; (c) screening the plurality of transformed plant cells for expression of the miRNA encoded by the vector; (d) selecting a transformed plant cell that expresses the miRNA; and (e) regenerating the plant from the transformed plant cell that expresses the miRNA, whereby expression of the plant gene is stably modulated.

In some embodiments of the disclosed methods, the modulating expression of a plant gene is inhibiting expression of the plant gene. In some embodiments, a method of stably inhibiting the expression of a gene in a plant cell comprises stably transforming the plant cell with a vector encoding a microRNA (miRNA) molecule, wherein the miRNA molecule comprises a nucleotide sequence at least 70% identical to a contiguous 17-24 nucleotide subsequence of the gene.

Any expression vector that can be used to express nucleic acids encoding miRNAs and/or siRNAs in plants can be used in conjunction with the presently disclosed subject matter. In some embodiments, the vector is an Agrobacterium binary vector. In some embodiments, the vector comprises (a) a promoter operatively linked to a nucleic acid molecule encoding the miRNA molecule; and (b) a transcription termination sequence.

The nucleic acids of the presently disclosed subject matter can be expressed from any promoter that shows activity in plants. In some embodiments, the promoter is a DNA-dependent RNA polymerase III promoter. In some embodiments, the promoter is selected from the group consisting of an RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, a tRNA gene promoter, and functional derivatives thereof. In some embodiments, the Arabidopsis thaliana 7SL RNA gene promoter comprises the sequence presented in SEQ ID NO: 164.

In some embodiments, promoters are chosen that direct tissue-, cell-type-, or stage-specific expression of the miRNAs. In some embodiments, the stable expression of the microRNA (miRNA) in the plant occurs in a location or tissue selected from the group consisting of epidermis, root, vascular tissue, xylem, meristem, cambium, cortex, pith, leaf, flower, seed, and combinations thereof.

In some embodiments of the disclosed methods, an miRNA is used to modulate the expression of a target gene. In some embodiments, the nucleic acid sequence encoding the microRNA (miRNA) molecule comprises a sense region, an antisense region, and a loop region, positioned in relation to each other such that upon transcription, a resulting RNA transcript is capable of forming a hairpin structure via intramolecular hybridization of the sense strand and the antisense strand. In some embodiments, the nucleic acid sequence encoding the microRNA (miRNA) molecule comprises a nucleotide sequence selected from the group consisting of SEQ ID NOs: 1-59, 1247-1295, and 1662-1712, and nucleotide sequences at least 70% identical to SEQ ID NOs: 1-59, 1247-1295, and 1662-1712.

The methods and compositions of the presently disclosed subject matter can be used to modulate the expression of a gene in any plant. In some embodiments, the plant is a dicot. In some embodiments, the plant is a monocot. In some embodiments, the plant is a tree. In some embodiments, the tree is an angiosperm. In some embodiments, the tree is a gymnosperm. In some embodiments, the tree is a member of the genus Populus. In some embodiments, the tree is a Populus trichocarpa tree. In some embodiments, the tree is a member of the genus Pinus. In some embodiments, the tree is a Pinus taeda tree.

The methods and compositions of the presently disclosed subject matter can be used to modulate the expression of any gene in a plant. In some embodiments, the plant gene has a nucleotide sequence comprising one of SEQ ID NOs: 176-781, 1376-1553, and 1749-1837, or a nucleotide sequence at least 80% identical to any of SEQ ID NOs: 176-781, 1376-1553, and 1749-1837. In some embodiments, the gene is selected from the group consisting of coniferaldehyde-5-hydroxylase (Cald5H), a lignin-related gene, a cellulose-related gene, a hemicellulose-related gene, a hormone-related gene, a stress-related gene, a disease-related gene, a growth-related gene, and a transcription factor gene. In some embodiments, the lignin-related gene is selected from the group consisting of sinapyl alcohol dehydrogenase (SAD), cinnamyl alcohol dehydrogenase (CAD), 4-coumarate:coenzyme A (CoA) ligase (4CL), cinnamoyl CoA O-methyltransferase (CCoAOMT), caffeate O-methyltransferase (COMT), ferulate-5-hydroxylase (F5H), cinnamate-4-hydroxylase (C4H), p-coumarate-3-hydroxylase (C3H), and phenylalanine ammonia lyase (PAL). In some embodiments, the cellulose-related gene is selected from the group consisting of cellulose synthase, cellulose synthase-like, glucosidase, glucan synthase, and sucrose synthase. In some embodiments, the hormone-related gene is selected from the group consisting of isopentyl transferase (ipt), gibberellic acid (GA) oxidase, auxin (AUX), and a rooting locus (ROL) gene.

The presently disclosed subject matter also provides vectors that can be used for performing the disclosed methods. In some embodiments, the vector for stably expressing a microRNA (miRNA) molecule in a plant comprises (a) a promoter operatively linked to a nucleic acid molecule encoding the miRNA molecule; and (b) a transcription termination sequence. In some embodiments, the vector is an Agrobacterium binary vector. In some embodiments, the Agrobacterium binary vector comprises a nucleic acid encoding a selectable marker operatively linked to a promoter.

The presently disclosed subject matter also provides kits comprising the disclosed vectors and at least one reagent for introducing the disclosed vectors into a plant cell. In some embodiments, the kit further comprises instructions for introducing the vector into a plant cell.

The presently disclosed subject matter also provides plant cells, transgenic plants, transgenic seed, and transgenic progeny comprising the disclosed vectors. In some embodiments, the plant cell is from a plant selected from the group consisting of poplar, pine, eucalyptus, sweetgum, other tree species, tobacco, Arabidopsis, rice, corn, wheat, cotton, potato, and cucumber.

The presently disclosed subject matter also provides a method for stably inhibiting the expression of a gene in a plant cell. In some embodiments, the method comprises stably transforming the plant cell with a vector encoding a microRNA (miRNA) molecule comprising a nucleotide sequence at least 70% identical to a contiguous 17-24 nucleotide subsequence of the gene.

The presently disclosed subject matter also provides a method for enhancing the expression of a gene in a plant cell. In some embodiments, the method comprises introducing into the plant cell a vector encoding a short interfering RNA (siRNA) molecule comprising a sequence that hybridizes under physiological conditions to a loop region or a stem region of a pre-microRNA that comprises a microRNA (miRNA) that modulates expression of the gene, thereby resulting in downregulation of expression of the miRNA and enhanced expression of the gene. In some embodiments, the microRNA (miRNA) comprises a nucleotide sequence selected from the group consisting of any of SEQ ID NOs: 1-59, 1247-1295, and 1662-1712 and nucleotide sequences at least 70% identical to any of SEQ ID NOs: 1-59, 1247-1295, and 1662-1712.

The presently disclosed subject matter also provides expression vectors for use with the disclosed methods. In some embodiments, an expression vector comprises a nucleic acid sequence encoding a microRNA (miRNA) molecule that stably downregulates expression of a plant gene. In some embodiments of the disclosed expression vectors, the nucleic acid sequence encoding the microRNA (miRNA) molecule comprises a nucleotide sequence selected from the group consisting of SEQ ID NOs: 1-59, 1247-1295, and 1662-1712 nucleotide sequences at least 70% identical to SEQ ID NOs: 1-59, 1247-1295, and 1662-1712. In some embodiments, the miRNA is at least 70% identical to about 17-24 contiguous nucleotides of a ribonucleic acid (RNA) transcribed from a gene selected from the group consisting of a lignin-related gene, a cellulose-related gene, a hemicellulose-related gene, a hormone-related gene, a stress-related gene, a disease-related gene, a growth-related gene, and a transcription factor gene. In some embodiments, the vector comprises a promoter for expressing the miRNA, a transcription termination sequence, and a cloning site between the promoter and the transcription termination sequence into which a nucleic acid molecule encoding the miRNA can be cloned. In some embodiments, the vector is a plasmid vector. In some embodiments, the vector further comprises a selectable marker. In some embodiments, the cloning site comprises a recognition sequence for at least one restriction enzyme that is not present elsewhere in the plasmid vector.

In some embodiments of the presently disclosed subject matter, the nucleic acid sequence encoding the microRNA (miRNA) comprises (a) a sense region; (b) an antisense region; and (c) a loop region, wherein the sense, antisense, and loop regions are positioned in relation to each other such that upon transcription, the resulting RNA molecule is capable of forming a hairpin structure via intramolecular hybridization of the sense strand and the antisense strand.

Accordingly, it is an object of the presently disclosed subject matter to provide a method for manipulating gene expression in plants using an miRNA-mediated approach. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting EXAMPLES.

### BRIEF DESCRIPTION OF THE DRAWINGS

FIG. 1 depicts a general structure for an siRNA molecule of the presently disclosed subject matter, wherein N is any nucleotide, provided that in the loop structure identified as N₅₋₉, all 5-9 nucleotides remain in a single-stranded conformation. Similarly, N₁₋₈ can be any sequence of 1-8 nucleotides or modified nucleotides, provided that the nucleotides remain in a single-stranded conformation in the siRNA molecule.

FIGS. 2A and 2B depict potential hairpin configurations for exemplary miRNA precursors. FIG. 2A depicts a miRNA precursor derived from the PtMIR 115a gene (SEQ ID NO: 95) comprising the nucleotide sequence of miRNA PtmiR 115 (SEQ ID NO: 24). FIG. 2B depicts an miRNA precursor derived from the PtMIR 61a gene (SEQ ID NO: 71) comprising the nucleotide sequence of miRNA PtmiR 61 (SEQ ID NO: 10). In each Figure, the miRNA sequence is underlined.

FIGS. 3A-3C depict potential hairpin configurations for a transcript of an exemplary miRNA precursor gene, PtMIR 156-1a (SEQ ID NO: 132). FIG. 3A depicts a hairpin configuration where the PtmiR 156-1 sequence (SEQ ID NO: 47 in RNA fdrm) is present in the 5′ arm of the hairpin. FIGS. 3B and 3C depict two hairpin configurations where the PtmiR 156-1 sequence (SEQ ID NO: 47 in RNA form) is present in the 3′ arm of the hairpin. FIG. 3B depicts a shorter stem-loop structure, and FIG. 3C depicts a longer (one is shorter (B) and another is longer stem-loop structure. FIG. 3C also shows the position of a 19-nucleotide side stem-loop, the nucleotides of which are not depicted for clarity. For each of FIGS. 3A-3C, the sequence of PtmiR 156-1 (SEQ ID NO: 47 in RNA form) is underlined.

FIG. 4 depicts Northern analysis of the expression of exemplary miRNAs in leaf (L), phloem (Ph), and developing xylem (X), tension wood (X_{TW}), and opposite wood (X_{OW}) stem xylems. 5S rRNA is included as an RNA quantity loading control.

FIGS. 5A-5E depict human H1 promoter-mediated siRNA silencing of GUS gene expression in transgenic tobacco. FIG. 5A depicts GUS staining of cross-sections of the stems, of the leaves, and of the roots of one month old siRNA-transgenic (GT1 and GT2) and GUS-expressing control (C) tobacco plants. FIG. 5B is a graph of GUS protein activity (Jefferson et al., 1987) in the leaves of control plants and of ten GT2 transgenic plants. Mean values were calculated from three independent measurements per line. FIG. 5C depicts a loading control for gel blot analysis of RNA transcript level using a 25S ribosomal RNA probe. FIG. 5D depicts the same gel blot as shown in FIG. 5C, but is used to characterize the level of GUS miRNA using a GUS cDNA probe. FIG. 5E depicts gel blot detection of siRNAs of about 21 nucleotides (nt) (position indicated) using a GUS cDNA probe as described in Hutvagner et al., 2000. RNA was isolated from a portion of the leaves used for the GUS protein activity assay depicted in FIG. 5B.

FIG. 6 depicts a schematic representation of plasmid pUCSL1. The plasmid contains a promoter fragment (289 basepairs; P_{7SL-RNA}) containing USE and TATA elements and a 3′-non-transcribed sequence (3′-NTS) fragment (267 basepairs) from the Arabidopsis thaliana At7SL4 gene, cloned into pUC19. Between the promoter and 3′-NTS sequences is a multiple cloning site (MCS) containing recognition sequences for Sma I, Bam HI, and Xba I, which can be used to clone siRNA sequences. The promoter:MCS:3′-NTS cassette can be excised from pUCSL1 using Eco RI and Hind III sites that are present at the 5′ and 3′ ends of the cassette, respectively.

FIG. 7 depicts a schematic representation of plasmid pSIT. The plasmid contains the promoter:MCS:3′-NTS cassette from pUCSL1 in the opposite transcriptional orientation and downstream of a selectable marker cassette, the latter consisting of a promoter, selectable marker gene, and terminator sequence. pSIT represents a binary vector transformation system mediated by Agrobacterium.

FIG. 8 depicts a representation of the multiple cloning site (MCS) of pSIT. Between the Sma I and Xba I sites of the MCS is cloned a sequence comprising 17-26 nt from the sense strand of the gene of interest, followed by a 9 nt spacer, and then the reverse complement of the 17-26 nt sequence (i.e., the antisense sequence cloned in the opposite direction). Downstream of the antisense sequence is the sequence TTTTTTT, which serves to terminate transcription from the promoter for siRNA transcription present in pSIT (see FIG. 7).

FIG. 9 depicts the preparation of siRNA expression constructs. The 19 nucleotide (nt) GUS gene-specific sequence (GT1 represented nucleotide positions 80-98 and GT2 89-107) separated by a 9 nt spacer from the reverse complement of the same sequence followed by a termination signal of five thymidines was cloned into pSUPER (available from OligoEngine, Inc., Seattle, Wash., United States of America) downstream of the H1 promoter (H1-P). The H1-P::GT expression construct was then excised and cloned into the binary vector pGPTV-HPT (Becker et al., 1992) to replace the pAnos-uidA fragment. The resulting vector, pGPH1-HPT, which contained a hygromycin phosphotransferase selectable marker gene (hpt), was then mobilized into Agrobacterium tumefaciens C58 for transforming tobacco. The predicted secondary siRNA structures of GT1 and GT2 are depicted at the bottom of the Figure. Considered in the 5′ to 3′ direction, FIG. 9 shows the sequences of GT1 and GT2 that form the hairpin as follows. For GT1, the hairpin is produced by the intramolecular hybridization of SEQ ID NO: 174 and SEQ ID NO: 175, with a 9 nt spacer between. For GT2, the hairpin is produced by the intramolecular hybridization of SEQ ID NO: 176 and SEQ ID NO: 177, with a 9 nt spacer between. FIG. 9 depicts these hairpins with the “top” strand in the 5′ to 3′ direction, and thus the “bottom” strand is depicted in the 3′ to 5′ direction.

### BRIEF DESCRIPTION OF THE SEQUENCE LISTING

The Sequence Listing discloses, inter alia, the sequences of various miRNAs, genes encoding miRNA precursors, and sequences derived from the genomes of Populus sp. and Pinus sp. that are targets for the disclosed miRNAs. While the sequences are presented in the form of DNA (i.e. with thymidine present instead of uracil), it is understood that the sequences are also intended to correspond to the RNA transcripts of these DNA sequences (i.e. with each T replaced by a U).

SEQ ID NOs: 1-59 and 1247-1295 are the nucleic acid sequences of various miRNAs from Populus trichocarpa.

SEQ ID NOs: 60-156 and 1296-1375 are the nucleic acid sequences of various miRNA precursor genes. The relationships between the sequences disclosed as SEQ ID NOs: 1-59 and 1247-1295 and those disclosed as 60-156 and 1296-1375 are presented Table 1 below.

SEQ ID NO: 155 is the nucleic acid sequence of a 5′-phosphorylated-3′-adaptor oligonucleotide used to clone a population of small RNAs predicted to include miRNAs.

SEQ ID NO: 156 is the nucleic acid sequence of a second adaptor molecule used during the isolation and cloning of small RNAs.

SEQ ID NOs: 157-159 are the nucleotide sequences of oligonucleotide primers used during the reverse transcription and amplification by PCR of the small RNAs to which the adaptors of SEQ ID NOs: 155 and 156 had been added.

SEQ ID NOs: 160 and 161 are primer sequences used to PCR-amplify a region of the Arabidopsis At7SL4 promoter.

SEQ ID NO: 162 is the nucleic acid sequence of the product of a PCR reaction using the primers identified in SEQ ID NOs: 160 and 161.

SEQ ID NOs: 163 and 164 are primer used to amplify the 3′-NTS of the At7SL4 gene.

SEQ ID NO: 165 is the nucleic acid sequence of the product of a PCR reaction using the primers identified in SEQ ID NOs: 163 and 164.

SEQ ID NOs: 166-171 are the sequences of complementary oligonucleotides that were used to generate siRNAs targeted to the GUS gene. Three different regions of the GUS gene were targeted. For the production of pGSGT1, SEQ ID NOs: 166 and 167 were hybridized to each other. For the production of pGSGT2, SEQ ID NOs: 168 and 169 were hybridized to each other. For the production of pGSGT3, SEQ ID NOs: 170 and 171 were hybridized to each other.

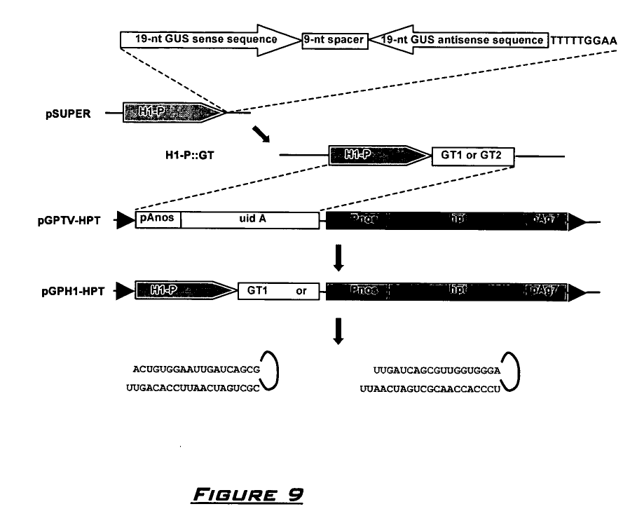

SEQ ID NOs: 172-175 are presented in FIG. 9, and correspond to the sense and antisense sequences for representative siRNA-like molecules targeting the GUS gene. SEQ ID NO: 172 is a nucleic acid sequence that corresponds to bases 80-98 of GENBANK® Accession No. AY100472, and is a sense strand sequence. SEQ ID NO: 173 is a nucleic acid sequence that hybridizes to SEQ ID NO: 174 and includes a one nucleotide 3′ overhang (U). SEQ ID NO: 174 is a nucleic acid sequence that corresponds to bases 89-107 of GENBANK® Accession No. AY100472, and is a sense strand sequence. SEQ ID NO: 175 is a nucleic acid sequence that hybridizes to SEQ ID NO: 174 and includes a two nucleotide 3′ overhangs (UU).

SEQ ID NOs: 176-781 and 1376-1553 are the nucleotide sequences of various genes and/or RNA transcripts (disclosed in “DNA form’” i.e. with T instead of U) identified in Populus spp. as targets for one or more of the miRNAs disclosed in SEQ ID NOs: 1-59 and 1247-1295.

SEQ ID NOs: 782-1246 are the amino acid sequences encoded by the nucleotide sequences disclosed in SEQ ID NOs: 176-781. Given that some of the nucleotide sequences disclosed in SEQ ID NOs: 176-781 encode the same amino acid sequence, there are fewer SEQ ID NOs. assigned to amino acid sequences than to nucleotide sequences. The relationships between the sequences disclosed as SEQ ID NOs: 176-1246 and 1376-1661 are presented Table 3 below.

SEQ ID NOs: 1662-1712 are the nucleic acid sequences of various miRNAs from Pinus taeda. SEQ ID NOs: 1713-1748 are the nucleic acid sequences of various miRNA precursor genes. The relationships between the sequences disclosed as SEQ ID NOs: 1662-1712 and 1713-1748 are presented Table 4 below.

SEQ ID NOs: 1749-1837 are the nucleotide sequences of various genes and/or RNA transcripts (disclosed in “DNA form’” i.e. with T instead of U) identified in Pinus sp. as targets for one or more of the miRNAs disclosed in SEQ ID NOs: 1662-1712.

SEQ ID NOs: 1838-1907 are the amino acid sequences encoded by the nucleotide sequences disclosed in SEQ ID NOs: 1749-1837. Given that some of the nucleotide sequences disclosed in SEQ ID NOs: 1749-1837 encode the same amino acid sequence, there are fewer SEQ ID NOs. assigned to amino acid sequences than to nucleotide sequences. The relationships between the sequences disclosed as SEQ ID NOs: 1749-1837 and 1838-1907 are presented Table 5 below.

### DETAILED DESCRIPTION

I. General Considerations

In studies of C. elegans development it was found that the lin-4 gene produced small RNAs of about 22 nucleotides (nt), instead of protein. It was further discovered that these small RNAs imperfectly paired to multiple sites in the 3′-untranslated region (3′-UTR) of lin-14 gene, mediating the translational repression of lin-14 message as part of the regulatory network that triggers the transition of developmental stages in the nematode (Lee R C et al., 1993; Wightman et al., 1993). These studies have led to the discovery of a new class of small, non-coding regulatory RNAs, termed microRNAs (miRNAs), and, thus, of a new paradigm of gene expression regulation in eukaryotes (Lagos-Quintana et al., 2001; Lau et al., 2001; Lee & Ambros, 2001).

In a recent review, Bartel summarized the current knowledge of the biogenesis and functions of miRNAs in eukaryotes (Bartel, 2004). Briefly, the miRNA gene is presumably processed by RNA polymerase II or RNA polymerase III to the primary miRNA stem-loop transcript, called pri-miRNA (Lee, N. S., et al., 2002). In mammals, the pri-miRNA is cleaved by the Drosha RNase III endonuclease at both stem strands near the stem-loop base, releasing an miRNA precursor (pre-miRNA) as an about 60-70 nt stem-loop RNA molecule (Lee, Y., et al., 2002; Zeng & Cullen, 2003). The pre-miRNA is then transported into the cytoplasm where it is cleaved at both stem strands by Dicer, also an RNase III endonuclease, liberating the loop portion of the pre-miRNA and the stem portion of the duplex that comprises the mature miRNA of about 22 nt and the similar size miRNA* fragment derived from the opposing arm of the pre-miRNA (Lau et al., 2001; Lagos-Quintana et al., 2002; Aravin et al., 2003; Lim et al., 2003b). In plants, the nuclear cleavage of the pri-miRNA is mediated by a Dicer-like protein, DCL1, having a similar functionality as mammal Drosha (Reinhart et al., 2002; Lim et al., 2003b; Lee, Y., et al., 2002; Lee, Y., et al., 2003). The resulting plant pre-miRNA stem-loop transcripts are, however, generally more variable in size, ranging from about 60 to about 300 nt (Bartel & Bartel, 2003; Bartel, 2004; Lim et al., 2003b). It is believed that in plants, DCL1 performs a second cut in the nucleus on the pre-miRNA to liberate the miRNA:miRNA* duplex (Reinhart et al., 2002; Lim et al., 2003b; Lee Y et al., 2002; Lee, Y., et al., 2003).

After the export of the miRNA:miRNA* duplex to the cytoplasm, the miRNA pathway in plants and mammals appears to be quite similar, both involving helicase-like protein-mediated unwinding of the duplex to release the single-stranded mature miRNA (Bartel & Bartel, 2003; Bartel, 2004; Rhoades et al., 2002). The mature miRNA then recruits a ribonucleoprotein complex known as the RNA-induced silencing complex (RISC), while the miRNA* appears to be degraded. The miRNA guides the RISC to identify target messages based on perfect or near perfect complementarity between the miRNA and the target miRNA. Once such an miRNA is found, an endonuclease within the RISC cleaves the miRNA at a site near the middle of the miRNA complementarity, resulting in gene silencing (Hutvágner et al., 2000; Elbashir et al., 2001a; Elbashir et al., 2001b; Llave et al., 2002; Kasschau et al., 2003). In general, the miRNA in RISC will direct cleavage of the target miRNA if the complementarity between the target miRNA and the miRNA is sufficiently high. If such complementarity is not sufficiently high, however, the miRNA will direct the repression of protein translation rather than target miRNA cleavage (Bartel & Bartel, 2003; Bartel, 2004).

This miRNA-guided gene silencing pathway is highly similar to the key steps of siRNA-mediated gene silencing known as posttranscriptional gene silencing (PTGS) in plants and RNA interference (RNAi) in animals (Hamilton & Baulcombe, 1999; Hutvágner & Zamore, 2002). There is a distinction between miRNA and siRNA, however. siRNAs, which can be exogenous sequences (for example, transgenes), mediate the silencing of the same genes from which they are derived. miRNAs, on the other hand, are typically endogenous and encoded by their own genes, and target different genes, setting up the gene regulation circuitry.

miRNAs have been cloned from various animals, including Drosophila melanogaster (Lagos-Quintana et al., 2001; Aravin et al., 2003), C. elegans (Lee & Ambros, 2001; Lim et al., 2003b; Ambros et al., 2003), fish (Lim et al., 2003a), mouse (Dostie et al., 2003; Houbaviy et al., 2003; Lagos-Quintana et al., 2003; Michael et al., 2003), and human (Lagos-Quintana et al., 2001; Mourelatos et al., 2002; Lagos-Quintana et al., 2003). Thus far, plant miRNAs have been isolated only from two non-woody plant species. The isolation is straightforward but the multitude of other small RNAs often complicates the initial classification (Llave et al., 2002; Park et al., 2002; Reinhart et al., 2002; Rhoades et al., 2002; Elbashir et al., 2001a; Ambros et al., 2003). Of the more than 300 small RNAs isolated from Arabidopsis, only about 20 unique sequences have been reliably identified as miRNAs (Reinhart et al., 2002; Rhoades et al., 2002; Bartel & Bartel, 2003). In rice, 20 unique miRNAs that met the relevant criteria were identified from over 200 small RNAs (Wang et al., 2004).

The more challenging task, however, is to identify targets of miRNAs in order to determine the functions of the miRNAs. The observation that Arabidopsis miR171 has perfect antisense complementarity to three miRNAs encoding SCARECROW-like transcription factors (Llave et al., 2002; Reinhart et al., 2002) led Rhoades et al. to successfully identify annotated Arabidopsis miRNAs having perfect or near perfect complementarity to the cloned Arabidopsis miRNAs (Rhoades et al., 2002). Seventy-four Arabidopsis target genes were identified, representing 61 unique miRNAs (Reinhart et al., 2002; Rhoades et al., 2002; Bartel & Bartel, 2003). When the same computational analysis was applied to animals, animal miRNAs had significantly lower miRNA hits, suggesting that perfect or near perfect miRNA:miRNA pairing might be specific to plants and, thus, that miRNA cleavage is the prevalent mechanism for miRNA-guided gene silencing in plants.

Furthermore, miRNA:miRNA pairings were conserved between Arabidopsis and rice (Reinhart et al., 2002; Rhoades et al., 2002; Bartel & Bartel, 2003; Wang et al., 2004). The most striking discovery was that, in the 61 predicted targets, 40 are known or putative transcription factors. Most of these transcription factors are known to regulate or are associated with development, suggesting that miRNAs might help coordinate a wide range of cell division and differentiation associated activities throughout the plant (Bartel & Bartel, 2003; Bartel, 2004).

The approach to gene function characterization through the use of microRNAs (miRNAs) offers the potential for agriculture and tree crop improvement. The ability to modulate the expression of genes involved in important biochemical pathways (for example, lignin synthesis) allows for the manipulation of the plant genome to produce plants with advantageous characteristics (for example, lower lignin content). miRNAs provide a general approach to modulating gene expression in plants that can potentially be applied to any plant gene. Thus, some embodiments the presently disclosed subject matter provide methods and compositions for modulating gene expression (for example, genes involved in lignin and/or cellulose synthesis) in plants (for example, trees, including but not limited to Populus trichocarpa and Pinus taeda).

II. Definitions

For convenience, certain terms employed in the specification, examples, and appended claims are collected here. While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, the articles “a”, “an”, and “the” are used herein to refer to one or to more than one (i.e., to at least one) of the grammatical object of the article. By way of example, “an element” refers to one element or more than one element.

As used herein, the term “about”, when referring to a value or to an amount of mass, weight, time, volume, concentration, or percentage is meant to encompass variations of in some embodiments ±20% or ±10%, in some embodiments ±5%, in some embodiments ±1%, in some embodiments ±0.5%, and in some embodiments ±0.1% from the specified amount, as such variations are appropriate to practice the presently disclosed subject matter. Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the terms “amino acid” and “amino acid residue” are used interchangeably and refer to any of the twenty naturally occurring amino acids, as well as analogs, derivatives, and congeners thereof; amino acid analogs having variant side chains; and all stereoisomers of any of the foregoing. Thus, the term “amino acid” is intended to embrace all molecules, whether natural or synthetic, which include both an amino functionality and an acid functionality and are capable of being included in a polymer of naturally occurring amino acids.

An amino acid is formed upon chemical digestion (hydrolysis) of a polypeptide at its peptide linkages. The amino acid residues described herein are in some embodiments in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature, abbreviations for amino acid residues are shown in tabular form presented hereinabove.

It is noted that all amino acid residue sequences represented herein by formulae have a left-to-right orientation in the conventional direction of amino terminus to carboxy terminus. In addition, the phrases “amino acid” and “amino acid residue” are broadly defined to include modified and unusual amino acids.

Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the term “cell” is used in its usual biological sense. In some embodiments, the cell is present in an organism, for example, a plant including, but not limited to poplar, pine, eucalyptus, sweetgum, and other tree species; tobacco; Arabidopsis; rice; corn; wheat; cotton; potato; and cucumber. The cell can be eukaryotic (e.g., a plant cell, such as a tobacco cell or a cell from a tree) or prokaryotic (e.g. a bacterium). The cell can be of somatic or germ line origin, totipotent, pluripotent, or differentiated to any degree, dividing or non-dividing. The cell can also be derived from or can comprise a gamete or embryo, a stem cell, or a fully differentiated cell.

As used herein, the terms “host cells” and “recombinant host cells” are used interchangeably and refer to cells (for example, plant cells) into which the compositions of the presently disclosed subject matter (for example, an expression vector) can be introduced. Furthermore, the terms refer not only to the particular plant cell into which an expression construct is initially introduced, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “gene” refers to a nucleic acid that encodes an RNA, for example, nucleic acid sequences including, but not limited to, structural genes encoding a polypeptide. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

As is understood in the art, a gene typically comprises a coding strand and a non-coding strand. As used herein, the terms “coding strand” and “sense strand” are used interchangeably, and refer to a nucleic acid sequence that has the same sequence of nucleotides as an miRNA from which the gene product is translated. As is also understood in the art, when the coding strand and/or sense strand is used to refer to a DNA molecule, the coding/sense strand includes thymidine residues instead of the uridine residues found in the corresponding miRNA. Additionally, when used to refer to a DNA molecule, the coding/sense strand can also include additional elements not found in the miRNA including, but not limited to promoters, enhancers, and introns. Similarly, the terms “template strand” and “antisense strand” are used interchangeably and refer to a nucleic acid sequence that is complementary to the coding/sense strand. It should be noted, however, that for those genes that do not encode polypeptide products (for example, an miRNA gene), the term “coding strand” is used to refer to the strand comprising the miRNA. In this usage, the strand comprising the miRNA is a sense strand with respect to the miRNA precursor, but it would be antisense with respect to its target RNA (i.e. the miRNA hybridizes to the target RNA because it comprises a sequence that is antisense to the target RNA).

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, ribonuclease activity. For example, the degree of complementarity between the sense and antisense strands of an miRNA precursor can be the same or different from the degree of complementarity between the miRNA-containing strand of an miRNA precursor and the target nucleic acid sequence. Determination of binding free energies for nucleic acid molecules is well known in the art. See e.g., Freier et al., 1986; Turner et al., 1987.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence. As miRNAs are about 17-24 nt, and up to 5 mismatches (e.g., 1, 2, 3, 4, or 5 mismatches) are tolerated during miRNA-directed modulation of gene expression, a percent complementarity of at least about 70% between a target RNA and an miRNA should be sufficient for the miRNA to modulate the expression of the gene from which the target RNA was derived.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA synthesis, processing, and transport, as well as polypeptide synthesis, transport, and post-translational modification of polypeptides. Additionally, processes that affect protein-protein interactions within the cell can also affect gene expression as defined herein.

However, in the case of genes that do not encode protein products, for example miRNA genes, the term “gene expression” refers to the processes by which a precursor miRNA is produced from the gene. Typically, this process is referred to as transcription, although unlike the transcription directed by RNA polymerase II for protein-coding genes, the transcription products of an miRNA gene are not translated to produce a protein. Nonetheless, the production of a mature miRNA from an miRNA gene is encompassed by the term “gene expression” as that term is used herein.

As used herein, the term “isolated” refers to a molecule substantially free of other nucleic acids, proteins, lipids, carbohydrates, and/or other materials with which it is normally associated, such association being either in cellular material or in a synthesis medium. Thus, the term “isolated nucleic acid” refers to a ribonucleic acid molecule or a deoxyribonucleic acid molecule (for example, a genomic DNA, cDNA, miRNA, miRNA, etc.) of natural or synthetic origin or some combination thereof, which (1) is not associated with the cell in which the “isolated nucleic acid” is found in nature, or (2) is operatively linked to a polynucleotide to which it is not linked in nature. Similarly, the term “isolated polypeptide” refers to a polypeptide, in some embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated”, when used in the context of an “isolated cell”, refers to a cell that has been removed from its natural environment, for example, as a part of an organ, tissue, or organism.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into a molecule, such as a polypeptide. Various methods of labeling polypeptides are known in the art and can be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for antibodies, metal binding domains, epitope tags). In some embodiments, labels are attached by spacer arms of various lengths to reduce potential steric hindrance.

As used herein, the term “modulate” refers to an increase, decrease, or other alteration of any, or all, chemical and biological activities or properties of a biochemical entity, e.g., a wild-type or mutant nucleic acid molecule. For example, the term “modulate” can refer to a change in the expression level of a gene or a level of an RNA molecule or equivalent RNA molecules encoding one or more proteins or protein subunits; or to an activity of one or more proteins or protein subunits that is upregulated or downregulated, such that expression, level, or activity is greater than or less than that observed in the absence of the modulator. For example, the term “modulate” can mean “inhibit” or “suppress”, but the use of the word “modulate” is not limited to this definition.

As used herein, the terms “inhibit”, “suppress”, “down regulate”, and grammatical variants thereof are used interchangeably and refer to an activity whereby gene expression or a level of an RNA encoding one or more gene products is reduced below that observed in the absence of a nucleic acid molecule of the presently disclosed subject matter. In some embodiments, inhibition with an miRNA molecule results in a decrease in the steady state expression level of a target RNA. In some embodiments, inhibition with an miRNA molecule results in an expression level of a target gene that is below that level observed in the presence of an inactive or attenuated molecule that is unable to downregulate the expression level of the target. In some embodiments, inhibition of gene expression with an miRNA molecule of the presently disclosed subject matter is greater in the presence of the miRNA molecule than in its absence. In some embodiments, inhibition of gene expression is associated with an enhanced rate of degradation of the miRNA encoded by the gene (for example, by miRNA-mediated inhibition of gene expression).

The term “modulation” as used herein refers to both upregulation (i.e., activation or stimulation) and downregulation (i.e., inhibition or suppression) of a response. Thus, the term “modulation”, when used in reference to a functional property or biological activity or process (e.g., enzyme activity or receptor binding), refers to the capacity to upregulate (e.g., activate or stimulate), downregulate (e.g., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like (naturally occurring or non-naturally occurring), or an extract made from biological materials such as bacteria, plants, fungi, or animal cells or tissues, that can be capable of causing modulation. Modulators can be evaluated for potential activity as inhibitors or activators (directly or indirectly) of a functional property, biological activity or process, or a combination thereof (e.g., agonist, partial antagonist, partial agonist, inverse agonist, antagonist, anti-microbial agents, inhibitors of microbial infection or proliferation, and the like), by inclusion in assays. In such assays, many modulators can be screened at one time. The activity of a modulator can be known, unknown, or partially known.

Modulators can be either selective or non-selective. As used herein, the term “selective” when used in the context of a modulator (e.g. an inhibitor) refers to a measurable or otherwise biologically relevant difference in the way the modulator interacts with one molecule (e.g. a target RNA of interest) versus another similar but not identical molecule (e.g. an RNA derived from a member of the same gene family as the target RNA of interest).

It must be understood that for a modulator to be considered a selective modulator, the nature of its interaction with a target need entirely exclude its interaction with other molecules related to the target (e.g. transcripts from family members other than the target itself). Stated another way, the term selective modulator is not intended to be limited to those molecules that only bind to miRNA transcripts from a gene of interest and not to those of related family members. The term is also intended to include modulators that can interact with transcripts from genes of interest and from related family members, but for which it is possible to design conditions under which the differential interactions with the targets versus the family members has a biologically relevant outcome. Such conditions can include, but are not limited to differences in the degree of sequence identity between the modulator and the family members, and the use of the modulator in a specific tissue or cell type that expresses some but not all family members. Under the latter set of conditions, a modulator might be considered selective to a given target in a given tissue if it interacts with that target to cause a biologically relevant effect despite the fact that in another tissue that expresses additional family members the modulator and the target would not interact to cause a biological effect at all because the modulator would be “soaked out” of the tissue by the presence of other family members.

When a selective modulator is identified, the modulator binds to one molecule (for example an miRNA transcript of a gene of interest) in a manner that is different (for example, stronger) from the way it binds to another molecule (for example, an miRNA transcript of a gene related to the gene of interest). As used herein, the modulator is said to display “selective binding” or “preferential binding” to the molecule to which it binds more strongly as compared to some other possible molecule to which the modulator might bind.

As used herein, the term “mutation” carries its traditional connotation and refers to a change, inherited, naturally occurring, or introduced, in a nucleic acid or polypeptide sequence, and is used in its sense as generally known to those of skill in the art.

The term “naturally occurring”, as applied to an object, refers to the fact that an object can be found in nature. For example, a polypeptide or polynucleotide sequence that is present in an organism (including bacteria) that can be isolated from a source in nature and which has not been intentionally modified by man in the laboratory is naturally occurring. It must be understood, however, that any manipulation by the hand of man can render a “naturally occurring” object an “isolated” object as that term is used herein.

As used herein, the terms “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids”, which comprise naturally occurring or modified nucleic acid bases attached to a polyamide backbone. Nucleic acids can be either single stranded or double stranded.

The term “operatively linked”, when describing the relationship between two nucleic acid regions, refers to a juxtaposition wherein the regions are in a relationship permitting them to function in their intended manner. For example, a control sequence “operatively linked” to a coding sequence can be ligated in such a way that expression of the coding sequence is achieved under conditions compatible with the control sequences, such as when the appropriate molecules (e.g., inducers and polymerases) are bound to the control or regulatory sequence(s). Thus, in some embodiments, the phrase “operatively linked” refers to a promoter connected to a coding sequence in such a way that the transcription of that coding sequence is controlled and regulated by that promoter. Techniques for operatively linking a promoter to a coding sequence are well known in the art; the precise orientation and location relative to a coding sequence of interest is dependent, inter alia, upon the specific nature of the promoter.

Thus, the term “operatively linked” can refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operatively linked. Techniques for operatively linking a promoter region to a nucleotide sequence are known in the art.

The term “operatively linked” can also refer to a transcription termination sequence that is connected to a nucleotide sequence in such a way that termination of transcription of that nucleotide sequence is controlled by that transcription termination sequence. In some embodiments, a transcription termination sequence comprises a sequence that causes transcription by an RNA polymerase III to terminate at the third or fourth T in the terminator sequence, TTTTTTT. Therefore the nascent small transcript has 3 or 4 U's at the 3′ terminus.

The phrases “percent identity” and “percent identical,” in the context of two nucleic acid or protein sequences, refer to two or more sequences or subsequences that have in some embodiments at least 60%, in some embodiments at least 700%, in some embodiments at least 80%, in some embodiments at least 85%, in some embodiments at least 90%, in some embodiments at least 95%, in some embodiments at least 98%, and in some embodiments at least 99% nucleotide or amino acid residue identity, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments the percent identity exists over at least about 150 residues. In some embodiments, the percent identity exists over the entire length of a given region, such as a coding region.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman, 1981, by the homology alignment algorithm described in Needleman & Wunsch, 1970, by the search for similarity method described in Pearson & Lipman, 1988, by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection. See generally, Ausubel et al., 1989.

One example of an algorithm that is suitable for determining percent sequence identity and sequence similarity is the BLAST algorithm, which is described in Altschul et al., 1990. Software for performing BLAST analyses is publicly available through the National Center for Biotechnology Information via the World Wide Web. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “substantially identical”, in the context of two nucleotide sequences, refers to two or more sequences or subsequences that have in some embodiments at least about 70% nucleotide identity, in some embodiments at least about 75% nucleotide identity, in some embodiments at least about 80% nucleotide identity, in some embodiments at least about 85% nucleotide identity, in some embodiments at least about 90% nucleotide identity, in some embodiments at least about 95% nucleotide identity, in some embodiments at least about 97% nucleotide identity, and in some embodiments at least about 99% nucleotide identity, when compared and aligned for maximum correspondence, as measured using one of the following sequence comparison algorithms or by visual inspection. In one example, the substantial identity exists in nucleotide sequences of at least 17 residues, in some embodiments in nucleotide sequence of at least about 18 residues, in some embodiments in nucleotide sequence of at least about 19 residues, in some embodiments in nucleotide sequence of at least about residues, in some embodiments in nucleotide sequence of at least about 21 residues, in some embodiments in nucleotide sequence of at least about 22 residues, in some embodiments in nucleotide sequence of at least about 23 residues, in some embodiments in nucleotide sequence of at least about 24 residues, in some embodiments in nucleotide sequence of at least about residues, in some embodiments in nucleotide sequence of at least about 26 residues, in some embodiments in nucleotide sequence of at least about 27 residues, in some embodiments in nucleotide sequence of at least about 30 residues, in some embodiments in nucleotide sequence of at least about 50 residues, in some embodiments in nucleotide sequence of at least about 75 residues, in some embodiments in nucleotide sequence of at least about 100 residues, in some embodiments in nucleotide sequences of at least about 150 residues, and in yet another example in nucleotide sequences comprising complete coding sequences. In some embodiments, polymorphic sequences can be substantially identical sequences. The term “polymorphic” refers to the occurrence of two or more genetically determined alternative sequences or alleles in a population. An allelic difference can be as small as one base pair. Nonetheless, one of ordinary skill in the art would recognize that the polymorphic sequences correspond to the same gene.

Another indication that two nucleotide sequences are substantially identical is that the two molecules specifically or substantially hybridize to each other under stringent conditions. In the context of nucleic acid hybridization, two nucleic acid sequences being compared can be designated a “probe sequence” and a “test sequence”. A “probe sequence” is a reference nucleic acid molecule, and a “‘test sequence” is a test nucleic acid molecule, often found within a heterogeneous population of nucleic acid molecules.

An exemplary nucleotide sequence employed for hybridization studies or assays includes probe sequences that are complementary to or mimic in some embodiments at least an about 14 to 40 nucleotide sequence of a nucleic acid molecule of the presently disclosed subject matter. In one example, probes comprise 14 to 20 nucleotides, or even longer where desired, such as 30, 40, 50, 60, 100, 200, 300, or 500 nucleotides or up to the full length of a given gene. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

By way of non-limiting example, hybridization can be carried out in 5×SSC, 4×SSC, 3×SSC, 2×SSC, 1×SSC, or 0.2×SSC for at least about 1 hour, 2 hours, 5 hours, 12 hours, or 24 hours (see Sambrook & Russell, 2001, for a description of SSC buffer and other hybridization conditions). The temperature of the hybridization can be increased to adjust the stringency of the reaction, for example, from about 25° C. (room temperature), to about 45° C., 50° C., 55° C., 60° C., or 65° C. The hybridization reaction can also include another agent affecting the stringency; for example, hybridization conducted in the presence of 50% formamide increases the stringency of hybridization at a defined temperature.

The hybridization reaction can be followed by a single wash step, or two or more wash steps, which can be at the same or a different salinity and temperature. For example, the temperature of the wash can be increased to adjust the stringency from about 25° C. (room temperature), to about 45° C., 50° C., 55° C., 60° C., 65° C., or higher. The wash step can be conducted in the presence of a detergent, e.g., SDS. For example, hybridization can be followed by two wash steps at 65° C. each for about 20 minutes in 2×SSC, 0.1% SDS, and optionally two additional wash steps at 65° C. each for about 20 minutes in 0.2×SSC, 0.1% SDS.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm ethylenediamine tetraacetic acid (EDTA) at 50° C. followed by washing in 2×SSC, 0.1% SDS at 50° C.; in some embodiments, a probe and test sequence hybridize in 7% sodium dodecyl sulfate (SDS), 0.5M NaPO₄, 1 mm EDTA at 50° C. followed by washing in 1×SSC, 0.1% SDS at 50° C.; in some embodiments, a probe and test sequence hybridize in 7% sodium dodecyl sulfate (SDS), 0.5M NaPO₄, 1 mm EDTA at 50° C. followed by washing in 0.5×SSC, 0.1% SDS at 50° C.; in some embodiments, a probe and test sequence hybridize in 7% sodium dodecyl sulfate (SDS), 0.5M NaPO₄, 1 mm EDTA at 50° C. followed by washing in 0.1×SSC, 0.1% SDS at 50° C.; in yet another example, a probe and test sequence hybridize in 7% sodium dodecyl sulfate (SDS), 0.5M NaPO₄, 1 mm EDTA at 50° C. followed by washing in 0.1×SSC, 0.1% SDS at 65° C.

Additional exemplary stringent hybridization conditions include overnight hybridization at 42° C. in a solution comprising or consisting of 50% formamide, 10× Denhardt's (0.2% Ficoll, 0.2% polyvinylpyrrolidone, 0.2% bovine serum albumin) and 200 mg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization can include hybridizing two nucleic acids in solution, or a nucleic acid in solution to a nucleic acid attached to a solid support, e.g., a filter. When one nucleic acid is on a solid support, a prehybridization step can be conducted prior to hybridization. Prehybridization can be carried out for at least about 1 hour, 3 hours, or 10 hours in the same solution and at the same temperature as the hybridization (but without the complementary polynucleotide strand).

Thus, upon a review of the present disclosure, stringency conditions are known to those skilled in the art or can be determined experimentally by the skilled artisan. See e.g., Ausubel et al., 1989; Sambrook & Russell, 2001; Agrawal, 1993; Tijssen, 1993; Tibanyenda et al., 1984; and Ebel et al., 1992.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

The term “phenotype” refers to the entire physical, biochemical, and physiological makeup of a cell or an organism, e.g., having any one trait or any group of traits. As such, phenotypes result from the expression of genes within a cell or an organism, and relate to traits that are potentially observable or assayable.

As used herein, the terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide”, and “peptide” are used interchangeably herein when referring to a gene product. The term “polypeptide” encompasses proteins of all functions, including enzymes. Thus, exemplary polypeptides include gene products, naturally occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. Further, fragments can include a sub-fragment of a specific region, which sub-fragment retains a function of the region from which it is derived.

As used herein, the term “primer” refers to a sequence comprising in some embodiments two or more deoxyribonucleotides or ribonucleotides, in some embodiments more than three, in some embodiments more than eight, and in some embodiments at least about 20 nucleotides of an exonic or intronic region. Such oligonucleotides are in some embodiments between ten and thirty bases in length.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species can be purified to essential homogeneity (contaminant species cannot be detected in the composition by conventional detection methods) wherein the composition consists essentially of a single species. A skilled artisan can purify a polypeptide of the presently disclosed subject matter using standard techniques for protein purification in light of the teachings herein. Purity of a polypeptide can be determined by a number of methods known to those of skill in the art, including for example, amino-terminal amino acid sequence analysis, gel electrophoresis, and mass-spectrometry analysis.

A “reference sequence” is a defined sequence used as a basis for a sequence comparison. A reference sequence can be a subset of a larger sequence, for example, as a segment of a full-length nucleotide or amino acid sequence, or can comprise a complete sequence. Generally, when used to refer to a nucleotide sequence, a reference sequence is at least 200, 300 or 400 nucleotides in length, frequently at least 600 nucleotides in length, and often at least 800 nucleotides in length. Because two proteins can each (1) comprise a sequence (i.e., a portion of the complete protein sequence) that is similar between the two proteins, and (2) can further comprise a sequence that is divergent between the two proteins, sequence comparisons between two (or more) proteins are typically performed by comparing sequences of the two proteins over a “comparison window” (defined hereinabove) to identify and compare local regions of sequence similarity.

The term “regulatory sequence” is a generic term used throughout the specification to refer to polynucleotide sequences, such as initiation signals, enhancers, regulators, promoters, and termination sequences, which are necessary or desirable to affect the expression of coding and non-coding sequences to which they are operatively linked. Exemplary regulatory sequences are described in Goeddel, 1990, and include, for example, the early and late promoters of simian virus 40 (SV40), adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. The nature and use of such control sequences can differ depending upon the host organism. In prokaryotes, such regulatory sequences generally include promoter, ribosomal binding site, and transcription termination sequences. The term “regulatory sequence” is intended to include, at a minimum, components the presence of which can influence expression, and can also include additional components the presence of which is advantageous, for example, leader sequences and fusion partner sequences.

In certain embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide. In some embodiments, a promoter sequence is a DNA-dependent RNA polymerase III promoter (e.g. a promoter for an H1, 5S, or U6 gene, or an Arabidopsis thaliana At7SL4 gene promoter, such as that disclosed as SEQ ID NO: 162). In some embodiments, a promoter sequence is selected from the group consisting of an adenovirus VA1 promoter sequence, a Vault promoter sequence, a telomerase RNA promoter sequence, and a tRNA gene promoter sequence. It is understood that the entire promoter identified for any promoter (for example, the promoters listed herein) need not be employed, and that a functional derivative thereof can be used. As used herein, the phrase “functional derivative” refers to a nucleic acid sequence that comprises sufficient sequence to direct transcription of another operatively linked nucleic acid molecule. As such, a “functional derivative” can function as a minimal promoter, as that term is defined herein.

Termination of transcription of a polynucleotide sequence is typically regulated by an operatively linked transcription termination sequence (for example, an RNA polymerase III termination sequence). In certain instances, transcriptional terminators are also responsible for correct mRNA polyadenylation. The 3′ non-transcribed regulatory DNA sequence includes from in some embodiments about 50 to about 1,000, and in some embodiments about 100 to about 1,000, nucleotide base pairs and contains plant transcriptional and translational termination sequences. Appropriate transcriptional terminators and those that are known to function in plants include the cauliflower mosaic virus (CaMV) ³⁵S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, a gamma coixin, oleosin 3, or other terminator from the genus Coix can be used. In some embodiments, an RNA polymerase III termination sequence comprises the nucleotide sequence TTTTTTT.

The term “reporter gene” refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, β-lactamase, phosphatases, peroxidases, proteases (tissue plasminogen activator or urokinase), and other enzymes whose function can be detected by appropriate chromogenic or fluorogenic substrates known to those skilled in the art or developed in the future.

As used herein, the term “sequencing” refers to determining the ordered linear sequence of nucleic acids or amino acids of a DNA, RNA, or protein target sample, using conventional manual or automated laboratory techniques.

As used herein, the term “substantially pure” refers to that the polynucleotide or polypeptide is substantially free of the sequences and molecules with which it is associated in its natural state, and those molecules used in the isolation procedure. The term “substantially free” refers to that the sample is in some embodiments at least 50%, in some embodiments at least 70%, in some embodiments 80% and in some embodiments 90% free of the materials and compounds with which is it associated in nature.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “target gene” refers to a gene expressed in a cell the expression of which is targeted for modulation using the methods and compositions of the presently disclosed subject matter. A target gene, therefore, comprises a nucleic acid sequence the expression level of which is downregulated by an miRNA. Similarly, the terms “target RNA” or “target mRNA” refers to the transcript of a target gene to which the miRNA is intended to bind, leading to modulation of the expression of the target gene. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus.

As used herein, the term “transcription” refers to a cellular process involving the interaction of an RNA polymerase with a gene that directs the expression as RNA of the structural information present in the coding sequences of the gene. The process includes, but is not limited to, the following steps: (a) the transcription initiation; (b) transcript elongation; (c) transcript splicing; (d) transcript capping; (e) transcript termination; (f) transcript polyadenylation; (g) nuclear export of the transcript; (h) transcript editing; and (i) stabilizing the transcript.

As used herein, the term “transcription factor” refers to a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of a “transcription factor for a gene” pertains to a factor that alters the level of transcription of the gene in some way.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell can express a recombinant form of a polypeptide of the presently disclosed subject matter.

The transformation of a cell with an exogenous nucleic acid (for example, an expression vector) can be characterized as transient or stable. As used herein, the term “stable” refers to a state of persistence that is of a longer duration than that which would be understood in the art as “transient”. These terms can be used both in the context of the transformation of cells (for example, a stable transformation), or for the expression of a transgene (for example, the stable expression of a vector-encoded miRNA) in a transgenic cell. In some embodiments, a stable transformation results in the incorporation of the exogenous nucleic acid molecule (for example, an expression vector) into the genome of the transformed cell. As a result, when the cell divides, the vector DNA is replicated along with plant genome so that progeny cells also contain the exogenous DNA in their genomes.

In some embodiments, the term “stable expression” relates to expression of a nucleic acid molecule (for example, a vector-encoded miRNA) over time. Thus, stable expression requires that the cell into which the exogenous DNA is introduced express the encoded nucleic acid at a consistent level over time. Additionally, stable expression can occur over the course of generations. When the expressing cell divides, at least a fraction of the resulting daughter cells can also express the encoded nucleic acid, and at about the same level. It should be understood that it is not necessary that every cell derived from the cell into which the vector was originally introduced express the nucleic acid molecule of interest. Rather, particularly in the context of a whole plant, the term “stable expression” requires only that the nucleic acid molecule of interest be stably expressed in tissue(s) and/or location(s) of the plant in which expression is desired. In some embodiments, stable expression of an exogenous nucleic acid is achieved by the integration of the nucleic acid into the genome of the host cell.

The term “vector” refers to a nucleic acid capable of transporting another nucleic acid to which it has been linked. One type of vector that can be used in accord with the presently disclosed subject matter is an Agrobacterium binary vector, i.e., a nucleic acid capable of integrating the nucleic acid sequence of interest into the host cell (for example, a plant cell) genome. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of plasmids. In the present specification, “plasmid” and “vector” are used interchangeably as the plasmid is the most commonly used form of vector. However, the presently disclosed subject matter is intended to include such other forms of expression vectors which serve equivalent functions and which become known in the art subsequently hereto.

The term “expression vector” as used herein refers to a DNA sequence capable of directing expression of a particular nucleotide sequence in an appropriate host cell, comprising a promoter operatively linked to the nucleotide sequence of interest which is operatively linked to transcription termination sequences. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression. The nucleotide sequence of interest, including any additional sequences designed to effect proper expression of the nucleotide sequences, can also be referred to as an “expression cassette”.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements. In some embodiments, a method of the presently disclosed subject matter employs a RNA polymerase III promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the cytomegalovirus (CMV) minimal promoter, the herpes simplex virus thymidine kinase (HSV-tk) minimal promoter, the simian virus 40 (SV40) minimal promoter, the human β-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operatively linked gene. For example, cell-type-specific or tissue-specific transcriptional regulatory elements can be added to minimal promoters to create recombinant promoters that direct transcription of an operatively linked nucleotide sequence in a cell-type-specific or tissue-specific manner. As used herein, the term “minimal promoter” also encompasses a functional derivative of a promoter disclosed herein, including, but not limited to an RNA polymerase III promoter (for example, an H1, 7SL, 5S, or U6 promoter), an adenovirus VA1 promoter, a Vault promoter, a telomerase RNA promoter, and a tRNA gene promoter.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types (in some embodiments, in all cell types) of an organism. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues or cell types of an organism but are inactive in some or all others tissues or cell types. Exemplary tissue-specific promoters include those promoters described in more detail hereinbelow, as well as other tissue-specific and cell-type specific promoters known to those of skill in the art.

When used in the context of a promoter, the term “linked” as used herein refers to a physical proximity of promoter elements such that they function together to direct transcription of an operatively linked nucleotide sequence

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element. In some embodiments, a transcriptional regulatory sequence is a transcription termination sequence, alternatively referred to herein as a transcription termination signal.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. In one example, a p-value less than or equal to 0.05, in some embodiments less than 0.01, in some embodiments less than 0.005, and in some embodiments less than 0.001, are regarded as significant.

As used herein, the phrase “target RNA” refers to an RNA molecule (for example, an mRNA molecule encoding a plant gene product) that is a target for modulation. Similarly, the phrase “target site” refers to a sequence within a target RNA that is “targeted” for cleavage mediated by an miRNA or siRNA construct that contains sequences within its antisense strand that are complementary to the target site. Also similarly, the phrase “target cell” refers to a cell that expresses a target RNA and into which an miRNA is intended to be introduced. A target cell is in some embodiments a cell in a plant. For example, a target cell can comprise a target RNA expressed in a plant.

An miRNA or an siRNA is “targeted to” an RNA molecule if it has sufficient nucleotide similarity to the RNA molecule that it would be expected to modulate the expression of the RNA molecule under conditions sufficient for the miRNA/siRNA and the RNA molecule to interact. In some embodiments, the interaction occurs within a plant cell. In some embodiments the interaction occurs under physiological conditions. As used herein, the phrase “physiological conditions” refers to in vivo conditions within a plant cell, whether that plant cell is part of a plant or a plant tissue, that plant cell is being grown in vitro. Thus, as used herein, the phrase “physiological conditions” refers to the conditions within a plant cell under any conditions that the plant cell can be exposed to, either as part of a plant or when grown in vitro.

As used herein, the phrase “detectable level of cleavage” refers to a degree of cleavage of target RNA (and formation of cleaved product RNAs) that is sufficient to allow detection of cleavage products above the background of RNAs produced by random degradation of the target RNA. Production of miRNA-mediated cleavage products from at least 1-5% of the target RNA is sufficient to allow detection above background for most detection methods.

The terms “microRNA” and “miRNA” are used interchangeably and refer to a nucleic acid molecule of about 17-24 nt that is produced from a pri-miRNA, a pre-miRNA, or a functional equivalent. As discussed in more detail herein, miRNAs are to be contrasted with siRNAs described hereinbelow, although in the context of exogenously supplied miRNAs and siRNAs, this distinction might be somewhat artificial. The distinction to keep in mind is that an miRNA is necessarily the product of nuclease activity on a hairpin molecule such as has been described herein, and an siRNA can be generated from a fully double-stranded RNA molecule or a hairpin molecule. Thus, while the distinction might be to some extent artificial, as used herein an miRNA is designed to hybridize to an mRNA derived from a gene of interest and an siRNA is designed to hybridize to an miRNA precursor such as a pri-miRNA or a pre-miRNA. miRNAs isolated from P. trichocarpa as disclosed herein are named using the general formula “PtmiR X”, where X is a number. This is in contrast to P. trichocarpa genes encoding miRNAs, which are named using the general formula “PtMIR X”, wherein X is a number sometimes followed by a lowercase letter. Thus, as referred to herein, miRNA names and miRNA-encoding gene names have the “MI” in lowercase and uppercase, respectively.

The terms “small interfering RNA”, “short interfering RNA”, and “siRNA” are used interchangeably and refer to a ribonucleic acid or a modified ribonucleic acid that is designed to hybridize to a single-stranded loop region of an miRNA precursor. As used herein, the term “miRNA precursor” refers to any ribonucleic acid derived from a DNA sequence encoding an miRNA. Exemplary miRNA precursors include pri-miRNAs and pre-miRNAs, although the term is not limited to only these species. In some embodiments, the siRNA comprises a single stranded polynucleotide having self-complementary sense and antisense regions, wherein either the sense or the antisense region comprises a sequence complementary to a loop region of a pri-miRNA or a pre-miRNA. In some embodiments, the siRNA comprises a single stranded polynucleotide having one or more loop structures and a stem comprising self complementary sense and antisense regions, wherein the antisense region comprises a sequence complementary to a loop region of a pri-miRNA or a pre-miRNA, and wherein the polynucleotide can be processed either in vivo or in vitro to generate an active siRNA capable of mediating cleavage of the miRNA precursor.

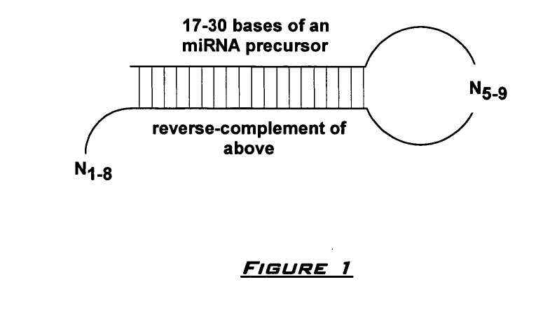

The methods of the presently disclosed subject matter can employ siRNA molecules of the general structure shown in FIG. 1, wherein N is any nucleotide, provided that in the loop structure identified as N₅₋₉ above, all 5-9 nucleotides remain in a single-stranded conformation. Similarly, N₁₋₈ can be any sequence of 1-8 nucleotides or modified nucleotides, provided that the nucleotides remain in a single-stranded conformation in the siRNA molecule. The duplex represented in FIG. 1 as 17-30 bases of an miRNA precursor” can be formed using any contiguous 17-30 base sequence of a transcription product of an miRNA-encoding nucleic acid sequence. In some embodiments, a contiguous 17-30 base sequence of a transcription product of an miRNA-encoding nucleic acid sequence comprises a subsequence that is predicted to hybridize to a single-stranded region of an miRNA precursor (for example, the loop region of a stem-loop conformation). In constructing an siRNA molecule of the presently disclosed subject matter, this 17-30 base sequence is followed (in a 5′ to 3′ direction) by 5-9 random nucleotides (N₅₋₉ above), the reverse-complement of the 17-30 base sequence, and finally 1-8 random nucleotides (N₁₋₈ above).

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, and recombinantly produced RNA. Thus, RNAs include, but are not limited to mRNA transcripts, miRNAs and miRNA precursors, and siRNAs. As used herein, the term “RNA” is also intended to encompass altered RNA, or analog RNA, which are RNAs that differ from naturally occurring RNA by the addition, deletion, substitution, and/or alteration of one or more nucleotides. Such alterations can include addition of non-nucleotide material, such as to the end(s) of the RNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 300 basepairs, and in some embodiments between 15 and about 60 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin” or a “stem-loop structure”.

III. Methods of Modulating Gene Expression

The presently disclosed subject matter provides in some embodiments methods for modulating gene expression in a plant. In some embodiments, the presently disclosed subject matter provides a method for stably modulating expression of a plant gene comprising (a) providing a vector encoding a microRNA (miRNA) targeted to the plant gene; and (b) transforming a plant cell with the vector, whereby stable expression of the miRNA in the plant cell is provided. Thus, in some embodiments the presently disclosed subject matter concerns stably transforming a plant cell (for example, a cell from a tree) with a vector encoding a miRNA under the control of a promoter (an other transcriptional regulatory elements as necessary, such as a transcription termination signal) that is functional in that cell. In some embodiments, an miRNA precursor is produced via the activity of the promoter in the plant cell, which is then processed using endogenous miRNA pathways to generate an miRNA target in the plant cell. This promoter can be capable of binding any RNA polymerase, including, for example, an RNA polymerase II and an RNA polymerase III. Representative promoters are disclosed hereinbelow, and include, but are not limited to an RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, a tRNA gene promoter, and functional derivatives thereof. These promoters can be naturally occurring or artificially produced. An exemplary promoter has the sequence disclosed in SEQ ID NO: 162.

In some embodiments, a method for stably modulating expression of a plant gene comprises (a) transforming a plurality of plant cells with a vector comprising a nucleic acid sequence encoding a microRNA (miRNA) operatively linked to a promoter and a transcription termination sequence; (b) growing the plant cells under conditions sufficient to select for a plurality of transformed plant cells that have integrated the vector into their genomes; (c) screening the plurality of transformed plant cells for expression of the miRNA encoded by the vector; (d) selecting a transformed plant cell that expresses the miRNA; and (e) regenerating the plant from the transformed plant cell that expresses the miRNA, whereby expression of the plant gene is stably modulated.

The presently disclosed subject matter also provides methods for enhancing the expression of a gene in a plant cell. In some embodiments, the method comprises introducing into the plant cell a vector encoding a short interfering RNA (siRNA) molecule comprising a sequence that hybridizes to a loop region, stem region, or antisense sequence of an miRNA of a pre-microRNA that comprises a microRNA (miRNA) that modulates expression of the gene, thereby resulting in downregulation of expression of the miRNA and enhanced expression of the gene.

In some embodiments, the disclosed methods are employed to modulate the expression of a gene in a tree cell. Representative, non-limiting tree species for which the disclosed methods can be employed include trees of the genus Populus and of the genus Pinus, including, but not limited to Populus trichocarpa and Pinus taeda.

IV. Target Genes

The presently disclosed subject matter provides methods for stably modulating expression of plant genes using miRNAs. The methods are applicable to any gene expressed in the plant. In some embodiments, the methods are used to modulate the expression of genes in trees. In some embodiments, the methods are used to modulate the expression of genes in members of the genus Populus, including, but not limited to Populus trichocarpa. In some embodiments, the methods are used to modulate the expression of genes in members of the genus Pinus, including, but not limited to Pinus taeda.

Representative P. trichocarpa miRNAs are presented in SEQ ID NOs: 1-59 and 1247-1295. These miRNA were identified using the techniques disclosed in Examples 1-6, and are summarized in Table 1. Additionally, using the techniques disclosed in the Examples, miRNA precursor sequences present in a representative plant, P. trichocarpa were identified, and these sequences (SEQ ID NOs: 60-156 and 1296-1375) are also summarized in Table 1. Further analysis of the P. trichocarpa genome revealed target genes that the miRNAs of SEQ ID NOs: 1-59 and 1247-1295 modulate, which are summarized in Table 2.

Representative Pinus taeda miRNAs are presented in SEQ ID NOs: 1662-1712. These miRNA were also identified using the techniques disclosed in Examples 1-6, and are summarized in Table 4. Additionally, using the techniques disclosed in the Examples, miRNA precursor sequences present in a second representative plant, Pinus taeda, were identified, and these sequences (SEQ ID NOs: 1713-1748) are also summarized in Table 4. Further analysis of the P. taeda genome revealed target genes that the miRNAs of SEQ ID NOs: 1662-1712 can modulate, which are also summarized in Table 2.

By comparing the nucleotide sequences of SEQ ID NOs: 1-59 and 1247-1295 to genomic and EST sequence data, plant gene sequences (for example, gene sequences from Populus sp. including, but not limited to Populus trichocarpa) that can be targeted by the miRNAs of SEQ ID NOs: 1-59 and 1247-1295 can be identified. In view of the ability of miRNAs to tolerate various degrees of mismatches between the miRNA molecule and the target molecule (for example, 1, 2, 3, 4 or 5 mismatches between the miRNA and the target), numerous particular target gene sequences were identified. These target gene sequences are presented in SEQ ID NOs: 176-781 and 1376-1553, and are summarized in Table 3.

Similarly, by comparing the nucleotide sequences of SEQ ID NOs: 1662-1712 to genomic and EST sequence data, plant gene sequences (for example, gene sequences from Pinus sp. including, but not limited to Pinus taeda) that can be targeted by the miRNAs of SEQ ID NOs: 1662-1712 can be identified. In view of the ability of miRNAs to tolerate various degrees of mismatches between the miRNA molecule and the target molecule (for example, 1, 2, 3, 4 or 5 mismatches between the miRNA and the target), numerous particular target gene sequences were identified. These target gene sequences are presented in SEQ ID NOs: 1749-1837, and are summarized in Table 5.

TABLE 1
Comparisons of P. trichocarpa and Arabidopsis miRNAs and miRNA Genes

| miRNA | Arabidopsis | | | | gene sequence |
|---|---|---|---|---|---|
| gene family | family name | Expressed | name of miRNA | name of gene | (SEQ ID NO:) |
| PtMIR 6 | | detected | PtmiR 6 | PtMIR 6 | 60 |
| | | | (SEQ ID NO: 1) | | |
| | | | PtmiR 6-1 | PtMIR 6-1 | 61 |
| | | | (SEQ ID NO: 2) | | |
| PtMIR 13 | AthMIR 408 | detected | PtmiR 13 | PtMIR 13 | 62 |
| | | | (SEQ ID NO: 3) | | |
| PtMIR 17 | | not detected | PtmiR 17 | PtMIR 17 | 63 |
| | | | (SEQ ID NO: 4) | | |
| | | | PtmiR 17-1 | PtMIR 17-1 | 64 |
| | | | (SEQ ID NO: 5) | | |
| | | | PtmiR 17-2 | PtMIR 17-2 | 65 |
| | | | (SEQ ID NO: 6) | | |
| PtMIR 29 | AthMIR 29 | detected | PtmiR 29 | PtMIR 29a | 66 |
| | | | (SEQ ID NO: 7) | PtMIR 29b | 67 |
| PtMIR 56 | AthMIR 168 | detected | PtmiR 56 | PtMIR 56a | 68 |
| | | | (SEQ ID NO: 8) | PtMIR 56b | 69 |
| | | | PtmiR 56-1 | PtMIR 56-1 | 70 |
| | | | (SEQ ID NO: 9) | | |
| PtMIR 61 | AthMIR 164 | detected | PtmiR 61 | PtMIR 61a | 71 |
| | | | (SEQ ID NO: 10) | PtMIR 61b | 72 |
| | | | | PtMIR 61c | 73 |
| | | | | PtMIR 61d | 74 |
| | | | | PtMIR 61e | 75 |
| | | | PtmiR 61-1 | PtMIR 61-1 | 76 |
| | | | (SEQ ID NO: 11) | | |
| PtMIR 69 | | detected | PtmiR 69 | PtMIR 69a | 77 |
| | | | (SEQ ID NO: 12) | PtMIR 69b | 78 |
| | | | PtmiR 69-1 | PtMIR 69-1 | 79 |
| | | | (SEQ ID NO: 13) | | |
| | | | PtmiR 69-2 | PtMIR 69-2 | 80 |
| | | | (SEQ ID NO: 14) | | |
| PtMIR 71 | AthMIR 319 | detected | PtmiR 71 | PtMIR 71a/ | 81 |
| | | | (SEQ ID NO: 15) | PtMIR 142-1a | |
| | | | | PtMIR 71b/ | 82 |
| | | | | PtMIR 142-1b | |
| | | | | PtMIR 71c/ | 83 |
| | | | | PtMIR 142-1c | |
| | | | | PtMIR 71d/ | 84 |
| | | | | PtMIR 142-1d | |
| | | | PtmiR 71-1 | PtMIR 71-1a/ | 85 |
| | | | (SEQ ID NO: 16) | PtMIR 142-2 | |
| | | | | PtMIR 71-1b/ | 86 |
| | | | | PtMIR 142-3a | |
| | | | | PtMIR 71-1c/ | 87 |
| | | | | PtMIR 142-3b | |
| | | | PtmiR 71-2 | PtMIR 71-2 | 88 |
| | | | (SEQ ID NO: 17) | | |
| | | | PtmiR 71-3 | PtMIR 71-3 | 89 |
| | | | (SEQ ID NO: 18) | | |
| PtMIR 73 | | detected | PtmiR 73 | PtMIR 73 | 90 |
| | | | (SEQ ID NO: 19) | | |
| | | | PtmiR 73-1 | PtMIR 73-1 | 91 |
| | | | (SEQ ID NO: 20) | | |
| PtMIR 104 | AthMIR 162 | detected | PtmiR 104 | PtMIR 104 | 92 |
| | | | (SEQ ID NO: 21) | | |
| PtMIR 109 | | detected | PtmiR 109 | PtMIR 109 | 93 |
| | | | (SEQ ID NO: 22) | | |
| | | | PtmiR 109-1 | PtMIR 109-1 | 94 |
| | | | (SEQ ID NO: 23) | | |
| PtMIR 115 | AthMIR 160 | detected | PtmiR 115 | PtMIR 115a | 95 |
| | | | (SEQ ID NO: 24) | PtMIR 115b | 96 |
| | | | | PtMIR 115c | 97 |
| | | | | PtMIR 115d | 98 |
| | | | PtmiR 115-1 | PtMIR 115-1 | 99 |
| | | | (SEQ ID NO: 25) | | |
| | | | PtmiR 115-2 | PtMIR 115-2 | 100 |
| | | | (SEQ ID NO: 26) | | |
| | | | PtmiR 115-3 | PtMIR 115-3a | 101 |
| | | | (SEQ ID NO: 27) | PtMIR 115-3b | 102 |
| | | | PtmiR 115-4 | PtMIR 115-4 | 103 |
| | | | (SEQ ID NO: 28) | | |
| PtMIR 122 | | detected | PtmiR 122 | PtMIR 122a | 104 |
| | | | (SEQ ID NO: 29) | PtMIR 122b | 105 |
| PtMIR 132 | | not detected | PtmiR 132 | PtMIR 132a | 106 |
| | | | (SEQ ID NO: 30) | PtMIR 132b | 107 |
| PtMIR 133 | similar to | detected | PtmiR 133 | | 108 |
| | AthMIR 172 | | (SEQ ID NO: 31) | | |
| | | | PtmiR 133-1 | PtMIR 133-1a | 109 |
| | | | (SEQ ID NO: 32) | PtMIR 133-1b | 110 |
| | | | PtmiR 133-2 | PtMIR 133-2 | 111 |
| | | | (SEQ ID NO: 33) | | |
| PtMIR 139 | | not detected | PtmiR 139 | PtMIR 139a | 112 |
| | | | (SEQ ID NO: 34) | PtMIR 139b | 113 |
| | | | | PtMIR 139c | 114 |
| | | | PtmiR 139-1 | PtMIR 139-1 | 115 |
| | | | (SEQ ID NO: 35) | | |
| | | | PtmiR 139-2 | PtMIR 139-2 | 116 |
| | | | (SEQ ID NO: 36) | | |
| | | | PtmiR 139-3 | PtMIR 139-3 | 117 |
| | | | (SEQ ID NO: 37) | | |
| PtMIR 140 | | detected | PtmiR 140 | PtMIR 140 | 118 |
| | | | (SEQ ID NO: 38) | | |
| PtMIR 142 | similar to | detected | PtmiR 142 | | 119 |
| | AthMIR 319 | | (SEQ ID NO: 39) | | |
| | | | PtmiR 142-1 | PtMIR 142-1a/ | 120 |
| | | | (SEQ ID NO: 40) | PtMIR 71-1a | |
| | | | | PtMIR 142-1b/ | 121 |
| | | | | PtMIR 71-1b | |
| | | | | PtMIR 142-1c/ | 122 |
| | | | | PtMIR 71-1c | |
| | | | | PtMIR 142-1d/ | 123 |
| | | | | PtMIR 71-1d | |
| | | | PtmiR 142-2 | PtMIR 142-2/ | 124 |
| | | | (SEQ ID NO: 41) | PtMIR 71-1a | |
| | | | PtmiR 142-3 | PtMIR 142-3a/ | 125 |
| | | | (SEQ ID NO: 42) | PtMIR 71-1b | |
| | | | | PtMIR 142-3b/ | 126 |
| | | | | PtMIR 71-1c | |
| PtMIR 145 | | not detected | PtmiR 145 | PtMIR 145 | 127 |
| | | | (SEQ ID NO: 43) | | |
| PtMIR 155 | | not detected | PtmiR 155 | PtMIR 155 | 128 |
| | | | (SEQ ID NO: 44) | | |
| | | | PtmiR 155-1 | PtMIR 155-1 | 129 |
| | | | (SEQ ID NO: 45) | | |
| PtMIR 156 | AthMIR 157 | detected | PtmiR 156 | PtMIR 156a | 130 |
| | | | (SEQ ID NO: 46) | PtMIR 156b | 131 |
| | | | | PtMIR 156c | 132 |
| | | | | PtMIR 156d | 133 |
| | | | Ptmir156-1 | PtMIR 156-1a | 134 |
| | | | (SEQ ID NO: 47) | PtMIR 156-1b | 135 |
| PtMIR 160 | | not detected | PtmiR 160 | PtMIR 160 | 136 |
| | | | (SEQ ID NO: 48) | | |
| | | | PtmiR 160-1 | PtMIR 160-1a | 137 |
| | | | (SEQ ID NO: 49) | PtMIR 160-1b | 138 |
| | | | | PtMIR 160-1c | 139 |
| | | | PtmiR 160-2 | PtMIR 160-2 | 140 |
| | | | (SEQ ID NO: 50) | | |
| | | | PtmiR 160-3 | PtMIR 160-3 | 141 |
| | | | (SEQ ID NO: 51) | | |
| | | | PtmiR 160-4 | PtMIR 160-4 | 142 |
| | | | (SEQ ID NO: 52) | | |
| PtMIR 172 | | not detected | PtmiR 172 | PtMIR 172 | 143 |
| | | | (SEQ ID NO: 53) | | |
| PtMIR 177 | | not detected | PtmiR 177 | PtMIR 177 | 144 |
| | | | (SEQ ID NO: 54) | | |
| PtMIR 180 | | | PtmiR 180 | PtMIR 180 | 145 |
| | | | (SEQ ID NO: 55) | | |
| PtMIR 181 | | not detected | PtmiR 181 | PtMIR 181 | 146 |
| | | | (SEQ ID NO: 56) | | |
| PtMIR 183 | similar to | detected | PtmiR 183 | PtMIR 183a | 147 |
| | AthMIR 170/171 | | (SEQ ID NO: 57) | PtMIR 183b | 148 |
| | | | | PtMIR 183c | 149 |
| | | | | PtMIR 183d | 150 |
| | | | | PtMIR 183e | 151 |
| | | | | PtMIR 183f | 152 |
| | | | | PtMIR 183g | 153 |
| | | | PtmiR 183-1 | PtMIR 183-1a | 154 |
| | | | (SEQ ID NO: 58) | PtMIR 183-1b | 155 |
| | | | PtmiR 183-2 | PtMIR 183-2 | 156 |
| | | | (SEQ ID NO: 59) | (antisense of | |
| | | | | PtMIR 183d) | |
| PtMIR184 | | N.A. | PtmiR184 | PtMIR184 | — |
| | | | (SEQ ID NO: 1247) | | |
| PtMIR185 | | N.A. | PtmiR185 | PtMIR185 | — |
| | | | (SEQ ID NO: 1248) | | |
| PtMIR186 | | N.A. | PtmiR186-1 | PtMIR186 | 1296 |
| | | | (SEQ ID NO: 1249) | | |
| | | | PtmiT186-2 | | |
| | | | (SEQ ID NO: 1250) | | |
| PtMIR241 | AthMIR397 | N.A. | PtmiR241 | PtMIR241 | 1297 |
| | | | (SEQ ID NO: 1251) | | |
| | | | PtmiR241-1 | PtMIR241-1 | 1298 |
| | | | (SEQ ID NO: 1252) | | |
| | | | PtmiR241-2 | PtMIR241-2 | 1299 |
| | | | (SEQ ID NO: 1253) | | |
| | | | PtmiR241-3 | PtMIR241-3 | 1300 |
| | | | (SEQ ID NO: 1254) | | |
| | | | PtmiR241-4 | PtMIR241-4 | 1301 |
| | | | (SEQ ID NO: 1255) | | |
| | | | PtmiR241-5 | PtMIR241-5 | 1302 |
| | | | (SEQ ID NO: 1256) | | |
| PtMIR244 | | N.A. | PtmiR244 | PtMIR244 | 1303 |
| | | | (SEQ ID NO: 1257) | | |
| | | | PtmiR244-1 | PtMIR244-1a | 1304 |
| | | | (SEQ ID NO: 1258) | PtMIR244-1b | 1305 |
| | | | PtmiR244-2 | PtMIR244-2 | — |
| | | | (SEQ ID NO: 1259) | | |
| PtMIR245 | | N.A. | PtmiR245 | PtMIR245 | — |
| | | | (SEQ ID NO: 1260) | | |
| | | | PtmiR245-1 | PtMIR245-1 | 1306 |
| | | | (SEQ ID NO: 1261) | | |
| PtMIR252 | AthMIR398 | N.A. | PtmiR252 | PtMIR252a | 1307 |
| | | | (SEQ ID NO: 1262) | PtMIR252b | 1308 |
| | | | PtmiR252-1 | PtMIR252-1 | 1309 |
| | | | (SEQ ID NO: 1263) | | |
| PtMIR253 | | N.A. | PtmiR253 | PtMIR253 | — |
| | | | (SEQ ID NO: 1264) | | |
| | | | PtmiR253-1 | PtMIR253-1 | 1310 |
| | | | (SEQ ID NO: 1265) | | |
| PtMIR255 | | N.A. | PtmiR255 | PtMIR255 | 1311 |
| | | | (SEQ ID NO: 1266) | | |
| PtMIR257 | | N.A. | PtmiR257 | PtMIR257a | 1312 |
| | | | (SEQ ID NO: 1267) | PtMIR257b | 1313 |
| | | | | PtMIR257c | 1314 |
| | | | | PtMIR257d | 1315 |
| | | | | PtMIR257e | 1316 |
| PtMIR274 | AthMIR166 | N.A. | PtmiR274 | PtMIR274a | 1317 |
| | | | (SEQ ID NO: 1268) | PtMIR274b | 1318 |
| | | | | PtMIR274c | 1319 |
| | | | | PtMIR274d | 1320 |
| | | | | PtMIR274e | 1321 |
| | | | | PtMIR274f | 1322 |
| | | | | PtMIR274g | 1323 |
| | | | | PtMIR274h | 1324 |
| | | | | PtMIR274i | 1325 |
| | | | | PtMIR274j | 1326 |
| | | | | PtMIR274k | 1327 |
| | | | | PtMIR274l | 1328 |
| | | | | PtMIR274m | 1329 |
| | | | PtmiR274-1 | PtMIR274-1a | 1330 |
| | | | (SEQ ID NO: 1269) | PtMIR274-1b | 1331 |
| | | | | PtMIR274-1c | 1332 |
| | | | PtmiR274-2 | PtMIR274-2 | 1333 |
| | | | (SEQ ID NO: 1270) | | |
| PtMIR275 | AthMIR167 | N.A. | PtmiR275 | PtMIR275a | 1334 |
| | | | (SEQ ID NO: 1271) | PtMIR275b | 1335 |
| | | | | PtMIR275c | 1336 |
| | | | | PtMIR275d | 1337 |
| | | | PtmiR275-1 | PtMIR275-1 | 1338 |
| | | | (SEQ ID NO: 1272) | | |
| | | | PtmiR275-2 | PtMIR275-2a | 1339 |
| | | | (SEQ ID NO: 1273) | PtMIR275-2b | 1340 |
| | | | PtmiR275-3 | PtMIR275-3 | 1341 |
| | | | (SEQ ID NO: 1274) | | |
| PtMIR277 | AthMIR396 | N.A. | PtmiR277 | PtMIR277a | 1342 |
| | | | (SEQ ID NO: 1275) | PtMIR277b | 1343 |
| | | | | PtMIR277c | 1344 |
| | | | | PtMIR277d | 1345 |
| | | | | PtMIR277e | 1346 |
| | | | PtmiR277-1 | PtMIR277-1a | 1347 |
| | | | (SEQ ID NO: 1276) | PtMIR277-1b | 1348 |
| | | | | PtMIR277-1c | 1349 |
| | | | | (antisense of | |
| | | | | PtMIR277a) | |
| | | | PtmiR277-2 | PtMIR277-2 | 1350 |
| | | | (SEQ ID NO: 1277) | (antisense of | |
| | | | | PtMIR277e) | |
| | | | PtmiR277-3 | PtMIR277-3 | — |
| | | | (SEQ ID NO: 1278) | | |
| PtMIR282 | AthMIR422 | N.A. | PtmiR282 | PtMIR282 | 1351 |
| | | | (SEQ ID NO: 1279) | | |
| | | | PtmiR282-1 | PtMIR282-1 | 1352 |
| | | | (SEQ ID NO: 1280) | | |
| PtMIR283 | | N.A. | PtmiR283 | PtMIR283 | — |
| | | | (SEQ ID NO: 1281) | | |
| PtMIR284 | AthMIR390 | N.A. | PtmiR284 | PtMIR284a | 1353 |
| | | | (SEQ ID NO: 1282) | PtMIR284b | 1354 |
| | | | | PtMIR284c | 1355 |
| | | | | PtMIR284d | 1356 |
| | | | PtmiR284-1 | PtMIR284-1a | 1357 |
| | | | (SEQ ID NO: 1283) | (antisense of | |
| | | | | PtMIR284b) | |
| | | | | PtMIR284-1b | 1358 |
| | | | | (antisense of | |
| | | | | PtMIR284d) | |
| PtMIR287 | | N.A. | PtmiR287 | PtMIR287 | 1359 |
| | | | (SEQ ID NO: 1284) | | |
| PtMIR291 | similar to | N.A. | PtmiR291 | PtMIR291a | 1360 |
| | AthMIR171 | | (SEQ ID NO: 1285) | PtMIR291b | 1361 |
| | | | | PtMIR291c | 1362 |
| PtMIR295 | | N.A. | PtmiR295 | PtMIR295 | — |
| | | | (SEQ ID NO: 1286) | | |
| PtMIR297 | | N.A. | PtmiR297 | PtMIR297a | 1363 |
| | | | (SEQ ID NO: 1287) | PtMIR297b | 1364 |
| PtMIR298 | | N.A. | PtmiR298 | PtMIR298 | 1365 |
| | | | (SEQ ID NO: 1288) | | |
| PtMIR302 | | N.A. | PtmiR302 | PtMIR302 | — |
| | | | (SEQ ID NO: 1289) | | |
| PtMIR304 | | N.A. | PtmiR304 | PtMIR304a | 1366 |
| | | | (SEQ ID NO: 1290) | PtMIR304b | 1367 |
| | | | | PtMIR304c | 1368 |
| | | | | PtMIR304d | 1369 |
| | | | | PtMIR304e | 1370 |
| | | | PtmiR304-1 | PtMIR304-1a | 1371 |
| | | | (SEQ ID NO: 1291) | PtMIR304-1b | 1372 |
| | | | PtmiR304-2 | PtMIR304-2 | 1373 |
| | | | (SEQ ID NO: 1292) | | |
| PtMIR310 | | N.A. | PtmiR310 | PtMIR310 | 1374 |
| | | | (SEQ ID NO: 1293) | | |
| PtMIR315 | | N.A. | PtmiR315 | PtMIR315 | — |
| | | | (SEQ ID NO: 1294) | | |
| | | | PtmiR315-1 | PtMIR315-1 | 1375 |
| | | | (SEQ ID NO: 1295) | | |

TABLE 2
Potential Targets of Populus trichopcarpa and Pinus taeda miRNAs

| P. trichopcarpa | A. thaliana | |
|---|---|---|
| miRNA ID | miRNA ID | Putative Function of Predicted Targets |
| PtMIR 133 | AtMIR 172 | APETAL2-like protein |
| PtMIR 104 | AtMIR 162 | DEAD/DEAH box helicase carpel factory/CAF identical to RNA |
| | | helicase/RNAseIII CAF protein |
| PtMIR 29 | AtMIR 159, 40 | MYB-related proteins |
| PtMIR 71/ | AtMIR 319 | MYB-related proteins |
| PtMIR 142 | | |
| PtMIR 183 | AtMIR 170, 171, 179 | scarecrow-like transcription factor |
| PtMIR 156 | AtMIR 157 | squamosa promoter binding protein |
| PtMIR 61 | AtMIR 164 | transcription activator contain NAC1 domain |
| PtMIR 115 | AtMIR 160 | transcriptional factor B3 family protein/similar to auxin-responsive factor |
| | | (ARF10) |
| PtMIR 56 | AtMIR 168 | AGRONAUTE |
| PtMIR 6 | — | (UVR8) UVB-resistance protein |
| PtMIR 13 | — | (ERD4) early-responsive to dehydration protein-related |
| | | plastocyanin |
| PtMIR 69 | — | pentatricopeptide (PPR) repeat-containing protein/F-box protein |
| | | UDP-glucoronosyl/UDP-glucosyl transferase family protein |
| | | protein kinase family protein |
| PtMIR 73 | — | disease resistance protein (TIR-NBS-LRR class) |
| PtMIR 109 | — | pentatricopeptide (PPR) repeat-containing protein |
| | | UDP-glucoronosyl/UDP-glucosyl transferase family protein |
| | | protein kinase family protein |
| PtMIR 122 | — | GARS domain transcription factor/similar to (RGL1) gibberellin regulatory |
| | | protein |
| PtMIR 139 | — | putative sulfate transporter |
| PtMIR 160 | — | disease resistance protein (TIR-NBS-LRR class) |
| PtMIR 180 | — | Intron of ubiquitin activating enzyme, putative (ECR1) |
| | | clathrin adaptor complex small chain family protein |
| PtMIR 181 | — | putative bifunctional aspartate kinase/homoserine dehydrogenase |
| | | lectin protein kinase family protein |
| PtMIR 172 | — | (CAD) cinnamyl-alcohol dehydrogenase |
| | | disease resistance protein-related LIM domain-containing protein |
| | | putative TCP family transcription factor |
| PtMIR 184 | — | lipase class 3 family protein |
| PtMIR 185 | — | UDP-glucoronosyl/UDP-glucosyl transferase |
| | | protein kinase family protein |
| | | mitogen-activated protein kinase |
| | | luminal binding protein 1 (BiP-1) |
| | | lipase class 3 family protein |
| | | ABC transporter family protein |
| PtMIR 186 | — | disease resistance protein |
| PtMIR 241 | — | Flavoprotein monooxygenase |
| | | laccase |
| | | pseudo-response regulator 5 |
| | | SPIa/RYanodine receptor (SPRY) domain-containing protein |
| | | polyphenol oxidase |
| | | SET domain-containing protein |
| | | KH domain-containing protein |
| PtMIR 245 | — | isoflavone reductase family protein |
| | | trehalose-6-phosphate phosphatase |
| PtMIR 252 | AthMIR 398 | selenium-binding protein, putative |
| PtMIR 255 | — | SEC14 cytosolic factor family protein |
| PtMIR 257 | — | GCN5-related N-acetyltransferase |
| | | gibberellin regulatory protein (RGL1) |
| | | homeodomain transcription factor (KNAT7) |
| PtMIR 274 | AthMIR 166 | homeobox-leucine zipper family protein |
| | | no apical meristem (NAM) family protein |
| PtMIR 275 | AthMIR 167 | auxin-responsive factor (ARF8) |
| | | Squamosa promoter binding protein |
| | | auxin-responsive factor (ARF6) |
| | | multi-copper oxidase |
| | | S-adenosylmethionine synthetase 2 (SAM2) |
| PtMIR 277 | AthMIR 396 | beta-fructofuranosidase, putative |
| | | DNAJ heat shock protein |
| | | PPR |
| | | trypsin and protease inhibitor family protein |
| | | calcium-binding EF hand family protein |
| | | calcium-transporting ATPase 4 |
| | | disease resistance protein |
| | | transcription activator GRL1 and GRL5 |
| | | expressed protein similar to auxin down-regulated protein ARG10 |
| | | malate synthase |
| | | protein kinase family protein |
| | | short vegetative phase protein (SVP) |
| | | SWAP (Suppressor-of-White-APricot)/surp domain-containing protein |
| PtMIR 282 | — | homeobox protein knotted-1 like 1 (KNAT1) |
| | | ribosomal protein L1 family protein |
| | | two-component responsive regulator family protein |
| PtMIR 283 | — | indigoidine synthase A family protein |
| | | pectate lyase family protein |
| | | eukaryotic release factor 1 family protein |
| PtMIR 284 | AthMIR 390 | auxin transport protein |
| | | leucine-rich repeat family protein |
| | | phosphate transporter (PT2) |
| | | subtilase family protein |
| PtMIR 287 | — | ankyrin repeat family protein |
| | | beta-fructosidase |
| | | disease resistance protein |
| | | leucine-rich repeat family protein |
| | | oxidoreductase, 2OG-Fe(II) oxygenase family protein |
| | | translationally controlled tumor family protein |
| PtMIR 291 | AthMIR 171 | acyl-CoA: 1-acylglycerol-3-phosphate acyltransferase |
| | | phosphatidylinositol-4-phosphate 5-kinase family protein |
| | | scarecrow transcription factor |
| PtMIR 295 | — | F-box family protein |
| PtMIR 298 | — | ATP-binding cassette transport protein |
| | | disease resistance protein |
| | | glutathione S-conjugate ABC transporter (MRP2) |
| PtMIR 302 | — | cytochrome P450 71B36 |
| | | rhomboid family protein |
| PtMIR 315 | — | BAG domain-containing protein |
| | | leucine-rich repeat family protein |
| LpMIR 100 | — | AMP-dependent synthetase |
| | | elongation factor Tu, putative/EF-Tu |
| | | expressed protein contains 3 transmembrane domains |
| | | peroxidase family protein similar to cationic peroxidase |
| LpMIR 119 | — | DEAD box RNA helicase, putative (RH20) |
| | | disease resistance protein |
| | | lipase |
| | | MYB transcription factor |
| | | ubiquitin activating enzyme |
| | | zinc finger (C2H2 type) |
| LpMIR 176 | — | ABC transporter family protein |
| | | AWPM-19-like membrane family protein |
| | | fructose-bisphosphate aldolase |
| | | osmotin-like protein (OSM34) |
| | | pyrophosphate-energized vacuolar membrane proton pump |
| LpMIR 178 | AthMIR 156 | F-box family protein (FBX1) E3 ubiquitin ligase |
| | | actin |
| | | aspartyl protease family protein |
| | | cellulose synthase |
| | | endo-(1,3)-alpha-glucanase |
| | | homeobox-leucine zipper protein 13 (HB-13) |
| | | lateral organ boundaries domain protein 4 (LBD4) |
| | | nitrate reductase 2 (NR2) |
| | | peptidyl-tRNA hydrolase |
| | | protein kinase family protein |
| | | Squamosa promoter binding protein |
| LpMIR 26 | — | disease resistance protein |
| | | leucine-rich repeat family protein |
| | | mob1/phocein family protein |
| | | oxidoreductase family protein |
| | | RuBisCO subunit binding-protein alpha subunit |
| LpMIR 27 | — | 3-deoxy-D-manno-octulosonic acid transferase |
| | | chlorophyll A-B binding family protein |
| | | hydrolase, alpha/beta fold family protein |
| | | nodulin MtN3 family protein |
| | | thioredoxin family protein |
| | | zinc finger (CCCH-type/C3HC4-type RING finger) family protein |
| LpMIR 28 | — | 60S ribosomal protein L24, putative |
| | | abscisic acid-responsive HVA22 family protein |
| | | aspartyl protease family protein |
| | | lipase class 3 family protein |
| | | microtubule organization 1 protein (MOR1) |
| | | SAR DNA-binding protein |
| LpMIR 7 | AthMIR 159, 319 | acyl-ACP thioesterase |
| | | ERF domain protein |
| | | MYB transcription factor |
| | | ethylene-responsive protein |
| | | ubiquitin carboxyl-terminal hydrolase family protein |
| | | 17.8 kDa class I heat shock protein |
| | | calcium-dependent protein kinase |
| | | GDSL-motif lipase/hydrolase family protein |
| LpMIR 77 | — | chloroplast nucleoid DNA-binding protein |
| | | protein kinase family protein |
| LpMIR 82 | — | disease resistance protein |
| | | leucine-rich repeat family protein |
| LpMIR 89 | — | protein phosphatase 2C family protein |
| | | sterol isomerase |
| LpMIR 9 | AthMIR 160 | auxin-responsive AUX/IAA family protein |
| | | transcriptional factor B3 family protein |
| LpMIR 95 | — | auxin-responsive GH3 protein |
| | | C2 domain-containing protein |
| | | MYB transcription factor |
| | | PQ-loop repeat family protein |
| | | glycosyl hydrolase family 29 |
| | | YbaK/prolyl-tRNA synthetase-related |
| | | zinc finger (C3HC4-type RING finger) |

TABLE 3

Populus trichocarpa miRNA Target Sequences

| | | | | |
|---|---|---|---|---|
| | | | Encoded | |
| miRNA gene | | SEQ ID | peptide | SEQ ID |
| family | Target sequence | NO: | sequence | NO: |

| | | | | |
|---|---|---|---|---|
| PtMIR 6 | ATAGATGCCTTGAAGGAGAGT | 176 | IDALKES | 782 |
| | CTGGATGCCTTCAGGGTGAGT | 177 | LDAFRVS | 783 |
| | TTGGATGCCCTGAGAGAGAGT | 178 | LDALRES | 784 |
| | TTGGAAGACTTGAAGGAGAGG | 179 | LEDLKER | 785 |
| | TTGGAACAATTGAGGGAGAGT | 180 | LEQLRES | 786 |
| | TTGGTAGCCTTGAGGGTGATT | 181 | LVALRVI | 787 |
| | ATGGAAGCATTGTGGGAGATT | 182 | MEALWEI | 788 |
| | AATGGAAGCATTGTGGGAGATTTT | 183 | NGSIVGDF | 789 |
| | GTGGATGGCTTGAGAGAGAGT | 184 | VDGLRES | 790 |
| | GTGGAAGCCTTGCGGGATAGT | 185 | VEALRDS | 791 |
| | GTTGAGGCCTTGAGGGAGGGT | 186 | VEALREG | 792 |
| | TGGAAACCTGCAGGGAGAGTT | 187 | WKPAGRV | 793 |
| PtMIR 13 | GCCAGGGTAGAGGCAGTGCTC | 188 | ARVEAVL | 794 |
| | GACAGGGAAGAGGCAATGGAT | 189 | DREEAMD | 795 |
| | TTCAGGGAAGAGGCAGTGCAA | 190 | FREEAVQ | 796 |
| | AAGACAGGGAAGAGGCAATGGATC | 191 | KTGKRQWI | 797 |
| | CGCCAGGGAAGATGCAGTGCGATC | 192 | RQGRCSAI | 798 |
| | AGCCAAGGATCAGGCAGTGCATGT | 193 | SQGSGSAC | 799 |
| | ACTCCAGTGAAGAGGCTGTGCATA | 194 | TPVKRLCI | 800 |
| | GTTCAGGGAAGAGGCAGTGCAATG | 195 | VQGRGSAM | 801 |
| PtMIR 29 | TTTGAGCTCCCTTCACTCCAATAT | 196 | FELPSLQY | 802 |
| | GGGAGCTCTCTTCAATCCATT | 197 | GSSLQSI | 803 |
| | AAGAGCTCCTTTCAATCCACT | 198 | KSSFQST | 804 |
| | AAGAGCTCTCTTCAATCCATT | 199 | KSSLQSI | 805 |
| | AAGAGCTCCCTTCAATCCACT | 200 | KSSLQST | 806 |
| | AAGACCTCCCTTCAATTCATA | 201 | KTSLQFI | 807 |
| | AAGACCTCCCTTCAATCCATA | 202 | KTSLQSI | 808 |
| | AAGACCTCCCTTCAATCCATT | 203 | KTSLQSI | 808 |
| | AAGACCTCCCTTCAATCCATG | 204 | KTSLQSM | 809 |
| | TTAGAGCTCCCTTCACTCCAATAT | 205 | LELPSLQY | 810 |
| | TTGGAGCTCCCTTCACTCCAATAT | 206 | LELPSLQY | 810 |
| | TTAGAGCTACCTTCAAACAAAAAT | 207 | LELPSNKN | 811 |
| | AGAGCTCCCTCCACTCCCAAC | 208 | RAPSTPN | 812 |
| | AGGGCTCAGTTCAATCCAAAC | 209 | RAQFNPN | 813 |
| | AGATCCTCCTTCAATCCAAAA | 210 | RSSFNPK | 814 |
| | TGGAGCTCCATTCGATCCAAA | 211 | WSSIRSK | 815 |
| PtMIR 61 | GCCTACGTGCCCTGCTTCTCCAAT | 212 | AYVPCFSN | 816 |
| | GAGCACGTGTCCTGTTTCTCCACC | 213 | EHVSCFST | 817 |
| | GAGCAAGTGCCCTGCTTCTCCATT | 214 | EQVPCFSI | 818 |
| | CTGCACGTGGCCTGCATCGCCATC | 215 | LHVACIAI | 819 |
| | CGAGCAAGTGCCCTGCTTCTCCAT | 216 | RASALLLH | 820 |
| | TCTCACGTGACCTGCTTCTCCAAT | 217 | SHVTCFSN | 821 |
| | AGCAAGTGCCCTGCTTCTCCA | 218 | SKCPASP | 822 |
| PtMIR 69 | TGCTTGATCAATGGGCTTTGTAAA | 219 | CLINGLCK | 823 |
| | ATCTTCATCAATGGGTACTGCAAG | 220 | IFINGYCK | 824 |
| | ATATTGATCAAGGGGCACTGTAAG | 221 | ILIKGHCK | 825 |
| | ATCTTAATCAATGGATGCTGTAAG | 222 | ILINGCCK | 826 |
| | ATACTAATCAATGGGCACTGTAAG | 223 | ILINGHCK | 827 |
| | ATATTGATCAACGGGCACTGTAAG | 224 | ILINGHCK | 827 |
| | ATCTTAATCAATGGATCTTGTAAG | 225 | ILINGSCK | 828 |
| | ATCTTAATCAATGGATATTGTAAG | 226 | ILINGYCK | 829 |
| | ATCTTAATTAATGGATATTGTAAG | 227 | ILINGYCK | 829 |
| | ACCTTGATCATTGGGCACTGTAAG | 228 | TLIIGHCK | 830 |
| | ACCTTAATCAATGGGCTCTGTAAA | 229 | TLINGLCK | 831 |
| | ACGTTAATTAATGGGCTCTGTAAA | 230 | TLINGLCK | 831 |
| | ACCTTAATCAATGGCCTCTGTACA | 231 | TLINGLCT | 832 |
| | ACCTTAATCAATGGGCTCGGTAAG | 232 | TLINGLGK | 833 |
| | ACCTTAATCAATTGGCTCTGTAAA | 233 | TLINWLCK | 834 |
| | ACCTTAACCAATGGGCTCTGTAAA | 234 | TLTNGLCK | 835 |
| PtMIR 71 | TTTGAGCTCCCTTCACTCCAA | 235 | FELPSLQ | 836 |
| | GGGGGCCCCCTTCAGTCCAGT | 236 | GGPLQSS | 837 |
| | GGGAGCTCTCTTCAATCCATT | 237 | GSSLQSI | 838 |
| | AAGAGCTCCCTTCAATCCACT | 238 | KSSLQST | 839 |
| | TTAGAGCTCCCTTCACTCCAA | 239 | LELPSLQ | 840 |
| | TTGGAGCTCCCTTCACTCCAA | 240 | LELPSLQ | 840 |
| | AGGGAACTCCATTCTGTCCAA | 241 | RELHSVQ | 841 |
| | AGGGGGCCCCCTTCAGTCCAG | 242 | RGPPSVQ | 842 |
| | TGGAGCTCCATTCGATCCAAA | 243 | WSSIRSK | 843 |
| PtMIR 73 | GGGCATGGGTGGAATAGGCAAGAC | 244 | GHGWNRQD | 844 |
| | GGCATTGCTGGAGTAGGGAAAACA | 245 | GIAGVGKT | 845 |
| | GGGATTGGTGGAGTAGGGAAGAAA | 246 | GIGGVGKK | 846 |
| | GGAATTGGTGGAGTTGGGAAGACA | 247 | GIGGVGKT | 847 |
| | GGGATTGGTGGAGTAGGGAAGACA | 248 | GIGGVGKT | 847 |
| | GGGATTGGTGGAGTTGGGAAGACA | 249 | GIGGVGKT | 847 |
| | GGGTTGTGTGGAGTAGGGAATAAG | 250 | GLCGVGNK | 848 |
| | GGGTTGTGTGGTGTAGGGAATAAG | 251 | GLCGVGNK | 848 |
| | GGGTTGAGTGGAGTAGGGAATAAG | 252 | GLSGVGNK | 849 |
| | GGTATGTGTGGAGTCGGGAAAACC | 253 | GMCGVGKT | 850 |
| | GGGATGGGAGAAGTTGGTAAAACG | 254 | GMGEVGKT | 851 |
| | GGAATGGGAGGCATAGGGAAAACA | 255 | GMGGIGKT | 852 |
| | GGAATGGGTGGAATAGGGAAGACA | 256 | GMGGIGKT | 852 |
| | GGAATGGGTGGTATAGGCAAAACA | 257 | GMGGIGKT | 852 |
| | GGCATGGGTGGAATAGGCAAGACA | 258 | GMGGIGKT | 852 |
| | GGCATGGGTGGTATAGGGAAAACA | 259 | GMGGIGKT | 852 |
| | GGGATGGGAGGAATAGGAAAGACA | 260 | GMGGIGKT | 852 |
| | GGGATGGGAGGTATAGGGAAGACA | 261 | GMGGIGKT | 852 |
| | GGGATGGGTGGAATAGGTAAGACG | 262 | GMGGIGKT | 852 |
| | GGAATGGGAGGGTTAGGGAAAACA | 263 | GMGGLGKT | 853 |
| | GGAATGGGGGGACTAGGGAAAACA | 264 | GMGGLGKT | 853 |
| | GGAATGGGGGGACTCGGGAAAACA | 265 | GMGGLGKT | 853 |
| | GGTATGGGTGGATTAGGTAAGACC | 266 | GMGGLGKT | 853 |
| | GGGATGGGAGGAGTTGGTAAATCC | 267 | GMGGVGKS | 854 |
| | GGGATGGGAGGAGTTGGTAAATCG | 268 | GMGGVGKS | 854 |
| | GGGATGGGGGGAGTTGGTAAATCC | 269 | GMGGVGKS | 854 |
| | GGAATGGGAGGAGTCGGTAAAACA | 270 | GMGGVGKT | 855 |
| | GGAATGGGAGGAGTGGGAAAAACC | 271 | GMGGVGKT | 855 |
| | GGAATGGGAGGAGTTGGTAAAACA | 272 | GMGGVGKT | 855 |
| | GGAATGGGAGGAGTTGGTAAAACG | 273 | GMGGVGKT | 855 |
| | GGAATGGGGGGAGTCGGGAAGACA | 274 | GMGGVGKT | 855 |
| | GGAATGGGGGGAGTCGGTAAAACA | 275 | GMGGVGKT | 855 |
| | GGAATGGGGGGAGTCGGTAAAACG | 276 | GMGGVGKT | 855 |
| | GGAATGGGGGGAGTTGGTAAAACA | 277 | GMGGVGKT | 855 |
| | GGAATGGGGGGAGTTGGTAAAACG | 278 | GMGGVGKT | 855 |
| | GGAATGGGTGGAGTTGGCAAAACG | 279 | GMGGVGKT | 855 |
| | GGCATGGGAGGAGTGGGTAAAACC | 280 | GMGGVGKT | 855 |
| | GGCATGGGGGGAGTTGGTAAAACG | 281 | GMGGVGKT | 855 |
| | GGGATGGGAGGAGTTGGGAAGACG | 282 | GMGGVGKT | 855 |
| | GGGATGGGAGGAGTTGGTAAAACA | 283 | GMGGVGKT | 855 |
| | GGGATGGGAGGGGTCGGTAAAACG | 284 | GMGGVGKT | 855 |
| | GGGATGGGAGGTGTGGGTAAAACA | 285 | GMGGVGKT | 855 |
| | GGGATGGGAGGTGTGGGTAAAACT | 286 | GMGGVGKT | 855 |
| | GGGATGGGCGGAGTGGGAAAGACC | 287 | GMGGVGKT | 855 |
| | GGGATGGGCGGAGTGGGAAAGACG | 288 | GMGGVGKT | 855 |
| | GGGATGGGCGGAGTGGGTAAGACC | 289 | GMGGVGKT | 855 |
| | GGGATGGGCGGAGTGGGTAAGACG | 290 | GMGGVGKT | 855 |
| | GGGATGGGGGGAGTTGGTAAAACA | 291 | GMGGVGKT | 855 |
| | GGGATGGGGGGAGTTGGTAAAACT | 292 | GMGGVGKT | 855 |
| | GGGATGGGTGGAGTGGGAAAGACG | 293 | GMGGVGKT | 855 |
| | GGGATGGGTGGTGTGGGGAAGACC | 294 | GMGGVGKT | 855 |
| | GGGATGAGAGGAGTAGGCAAGAAA | 295 | GMRGVGKK | 856 |
| | ATGGGATTGGTGGAGTTGGGAAGA | 296 | MGLVELGR | 857 |
| PtMIR 104 | CACTGGATGCAGAGCTTTATTAAA | 297 | HWMQSFIK | 858 |
| | CTGGATGCAGAGGTATATCAA | 298 | LDAEVYQ | 859 |
| | CTGGATCCAGAGTATTATCGA | 299 | LDPEYYR | 860 |
| PtMIR 109 | GCTATGCAAAGAAGGATTTCAACC | 300 | AMQRRIST | 861 |
| | TGCTATGCAAAGAAGGATTTCAAC | 301 | CYAKKDFN | 862 |
| | CTATGCAAAGAAGGATTTCAA | 302 | LCKEGFQ | 863 |
| | CTTTGCAAAGAAGGACTAATA | 303 | LCKEGLI | 864 |
| | CTTTGCAAAGAAGGATTGCTA | 304 | LCKEGLL | 865 |
| | CTTTGTAAAGAAGGATTATTA | 305 | LCKEGLL | 865 |
| | CTTTGTAAAGAAGGATTGTTA | 306 | LCKEGLL | 865 |
| | CTTTGCAAAGAAGGATTGGTA | 307 | LCKEGLV | 866 |
| | CTTTGCAAAGTAAGATTACAA | 308 | LCKVRLQ | 867 |
| | CTTTGCAGAGAAGGATTGCTA | 309 | LCREGLL | 868 |
| | CTTTGCAGAGAGGGATTGCTA | 310 | LCREGLL | 869 |
| | CTTTGCAGAGAAGGATCAATA | 311 | LCREGSI | 870 |
| | CTTTGCAGAGAAGGATCACTA | 312 | LCREGSL | 871 |
| | AATTTGGAAAGAAGTATTACTATT | 313 | NLERSITI | 872 |
| | CCTTTGCAAAGTAAGATTACAAGT | 314 | PLQSKITS | 873 |
| | TCTATCCAAAAAAGGATTACTAGC | 315 | SIQKRITS | 874 |
| | ACTTTGCAGAGAGGGATTGCTAGA | 316 | TLQRGIAR | 875 |
| | ACTTTGCAGAGAAGGATTGCTAGA | 317 | TLQRRIAR | 876 |
| PtMIR 115 | GCAGGCATACAGGGAGCCAGGCAT | 318 | AGIQGARH | 877 |
| | GCTGGCATGCAGGGAGCCAGGCAT | 319 | AGMQGARH | 878 |
| | GCTGGCATGCAGGGAGCCAGGCAA | 320 | AGMQGARQ | 879 |
| | TTGGCATACATGGACCCAGGAAGG | 321 | LAYMDPGR | 880 |
| PtMIR 122 | TTTTGGAAGCATCTGACGGAGTTT | 322 | FWKHLTEF | 881 |
| | TTGGATGCTTCTGAGCGAGAT | 323 | LDASERD | 882 |
| | TTGGAAGCCTTTGAGGGAGAG | 324 | LEAFEGE | 883 |
| | GTTTGGAAAGCACTGAGGGAGATT | 325 | VWKALREI | 884 |
| PtMIR 133 | GCTGCAGCATCATCAGGATTCCAA | 326 | AAASSGFQ | 885 |
| | GCTGCAGCATCATCAGGATTCCnn | 327 | AAASSGFX | 886 |
| | TGCTGCAGGATCATCAGGATTCCA | 328 | CCSIIRIP | 887 |
| | ATGCTGCAGCATCATCAGGATTCC | 329 | MLQHHQDS | 888 |
| PtMIR 139 | GTGCTTAAAAATAGAAGACACATCAAT | 330 | VLKNRRHIN | 889 |
| PtMIR 142 | GCAAAGGACCACTCTTCAGTCCAA | 331 | AKDHSSVQ | 890 |
| | AAGTTGGAGCTCCCTTCACTCCAA | 332 | KLELPSLQ | 891 |
| | AATAAGAGCTCCCTTCAATCCACT | 333 | NKSSLQST | 892 |
| PtMIR 156 | GCATGCTCTCTCTCTTCTGTCAAA | 334 | ACSLSSVK | 893 |
| | TGTGCTCTCTCTCTTCTGTCAAAT | 335 | CALSLLSN | 894 |
| | TGTGCTCTCTCTCTTCTGTCATCA | 336 | CALSLLSS | 895 |
| | TGTGCTCGCTCTCTTCTGTCATGC | 337 | CARSLLSC | 896 |
| | TGTGGTCTCTATATTCTGTCTAAG | 338 | CGLYILSK | 897 |
| | GATTGCTCTCTCTCTTCTGTCATC | 339 | DCSLSSVI | 898 |
| | CATGCTCTCTCTCTTCTGTCAATC | 340 | HALSLLSI | 899 |
| | CCTGCTCTCTGTCATCTGACAATC | 341 | PALCHLTI | 900 |
| | CGTGCTCTCTCTCTTCTGTCATCT | 342 | RALSLLSS | 901 |
| | CGTGCTCTCTCTCTTCTGTCAACC | 343 | RALSLLST | 902 |
| | GTGTTCTCTTTCTTCTGCCAA | 344 | VFSFFCQ | 903 |
| PtMIR 172 | GCGGAAGGGGAGAGGAAGGAA | 345 | AEGERKE | 904 |
| | GCGGAATGGGAGGAGAAGAGG | 346 | AEWEEKR | 905 |
| | GCCGAATGGGAGGAATGGGTA | 347 | AEWEEWV | 906 |
| | GCAATGGAAGAAGTAGGC | 348 | AMEEVG | 907 |
| | GCAATGGAAGGATTAGGA | 349 | AMEGLG | 908 |
| | GCAATGGGAGGGTTAGGT | 350 | AMGGLG | 909 |
| | GCAATGCAAGGAGTAGGA | 351 | AMQGVG | 910 |
| | GCGGTATGGGTGGAGGAGGAC | 352 | AVWVEED | 911 |
| | TGTGAATGGGAGAAGGAGGTA | 353 | CEWEKEV | 912 |
| | TGTAATGGGAAGAGTGGT | 354 | CNGKSG | 913 |
| | GATGAAGGGGAGGAGGAGGAG | 355 | DEGEEEE | 914 |
| | GATGAATGGGAGAAGTGGGTG | 356 | DEWEKWV | 915 |
| | GAGGACTGGGATGAGGAGGAG | 357 | EDWDEEE | 916 |
| | GAGGATTGGGATGAGGAGGAA | 358 | EDWDEEE | 916 |
| | GAGGATTGGGATGAGGAGGGA | 359 | EDWDEEG | 917 |
| | GAGGACTGGGACGAGCAGGCA | 360 | EDWDEQA | 918 |
| | GAGGATTGGGGGGAGTATGTT | 361 | EDWGEYV | 919 |
| | GAGGAGGAGGAGGAGGAGGAT | 362 | EEEEEED | 920 |
| | GAGGAAGAGGAGGAGGAGGAA | 363 | EEEEEEE | 921 |
| | GAGGAAGAGGAGGAGGAGGAG | 364 | EEEEEEE | 921 |
| | GAGGAGGAGGAGGAGGAGGAG | 365 | EEEEEEE | 921 |
| | GAGGAAGAGGAGGAGAAGGCG | 366 | EEEEEKA | 922 |
| | GAGGAGGGGGAGGAGGAGGAG | 367 | EEGEEEE | 923 |
| | GAGGAAGGGGAGGAGGAGCCG | 368 | EEGEEEP | 924 |
| | GAAGAAGGGGAGGAGTATGAA | 369 | EEGEEYE | 925 |
| | GAGGAATTGGAGGCGTTGGAT | 370 | EELEALD | 926 |
| | GAGGAGTTGGAGGAGGAGGCG | 371 | EELEEEA | 927 |
| | GAGGAAATGGAGGAGAAGGCT | 372 | EEMEEKA | 928 |
| | GAGGAAATGGAGGAGAAGGAA | 373 | EEMEEKE | 929 |
| | GAGGAACGGGAGGATTTGGCC | 374 | EEREDLA | 930 |
| | GAGGAGAGGGAGGAGGAGGAG | 375 | EEREEEE | 931 |
| | GAGGAAGTGGAGGAAGAGGAA | 376 | EEVEEEE | 932 |
| | GAGGAATGGGAGGAGGAAAAC | 377 | EEWEEEN | 933 |
| | GAGGAATGGGAGGAGTTCAGA | 378 | EEWEEFR | 934 |
| | GAGGAATGGGAGGAGTTTAGA | 379 | EEWEEFR | 934 |
| | GAGGAATGGGAGGAGAAGCAC | 380 | EEWEEKH | 935 |
| | GAGGAATGGGAGGAGAAAAAC | 381 | EEWEEKN | 936 |
| | GAGGAATGGGAGGAGAAGAAC | 382 | EEWEEKN | 936 |
| | GAAGAATGGGAGGAATACGGA | 383 | EEWEEYG | 937 |
| | GAGGAATGGGAGCAGCTGGTT | 384 | EEWEQLV | 938 |
| | GAAGGATGGGAGGAGTATGAA | 385 | EGWEEYE | 939 |
| | GAGGGATGGGAGAAGGAGGCT | 386 | EGWEKEA | 940 |
| | GAAAAGGGAGGACTAGGG | 387 | EKGGLG | 941 |
| | GAGAAATGGGAGGAGCAGCAG | 388 | EKWEEQQ | 942 |
| | GAAATGGGAGGAGCAGCA | 389 | EMGGAA | 943 |
| | GAAATGGGAGGGGTAGCA | 390 | EMGGVA | 944 |
| | GAAATGGGACTTGTAGGT | 391 | EMGLVG | 945 |
| | GAAATGCGAGGATTAGGT | 392 | EMRGLG | 946 |
| | GAAATGAGAGGAGTAAGC | 393 | EMRGVS | 947 |
| | GAGAATGCAAGGAGAAGG | 394 | ENARRR | 948 |
| | GAGAATTGGAGGAGAAGG | 395 | ENWRRR | 949 |
| | GAGCAATGGCAGGAGGAGGAT | 396 | EQWQEED | 950 |
| | GGAGATGGGAGGAGTAAG | 397 | GDGRSK | 951 |
| | GGGGAGGGGGAGGAGGAGGAG | 398 | GEGEEEE | 952 |
| | GGAGAGTGGGATGAGGAGGAG | 399 | GEWDEEE | 953 |
| | GGGGAATGGGACCAGAAGGGT | 400 | GEWDQKG | 954 |
| | GGGGAATGGGAGGAGGACTGG | 401 | GEWEEDW | 955 |
| | GGAGGAGGAGGAGTAGGA | 402 | GGGGVG | 956 |
| | GGGCATGGGTGGAATAGG | 403 | GHGWNR | 957 |
| | GGCATTGCTGGAGTAGGG | 404 | GIAGVG | 958 |
| | GGGATTGAGAGGAGTAGA | 405 | GIERSR | 959 |
| | GGAATAGGAGCAGCAGGT | 406 | GIGAAG | 960 |
| | GGAATAGGAGGAGCTGGT | 407 | GIGGAG | 961 |
| | GGAATTGGAGGAGGAGAG | 408 | GIGGGE | 962 |
| | GGAATTGGCGGAATAGGC | 409 | GIGGIG | 963 |
| | GGAATTGGAGGAAAAGGA | 410 | GIGGKG | 964 |
| | GGAATTGGAGGAAAAGGC | 411 | GIGGKG | 964 |
| | GGAATAGGTGGAGTTGGA | 412 | GIGGVG | 965 |
| | GGAATTGGCGGCGTAGGT | 413 | GIGGVG | 965 |
| | GGAATTGGTGGAGTTGGA | 414 | GIGGVG | 965 |
| | GGAATTGGTGGAGTTGGG | 415 | GIGGVG | 965 |
| | GGAATTGGTGGAGTTGGT | 416 | GIGGVG | 965 |
| | GGGATTGGTGGAGTAGGG | 417 | GIGGVG | 965 |
| | GGGATTGGTGGAGTTGGG | 418 | GIGGVG | 965 |
| | GGGATTGGGAGGAGTTGC | 419 | GIGRSC | 966 |
| | GGAATCGGAAGCGTCGGT | 420 | GIGSVG | 967 |
| | GGAATTAGAGGAGGAGGA | 421 | GIRGGG | 968 |
| | GGAATCGTAGGAGTGGGA | 422 | GIVGVG | 969 |
| | GGAAAAGCAGGAGTAGGT | 423 | GKAGVG | 970 |
| | GGCAAGGGAGAAGTAGTT | 424 | GKGEVV | 971 |
| | GGAAAGGGAGGATTTGGA | 425 | GKGGFG | 972 |
| | GGAAAGGGAGGGGGAGGG | 426 | GKGGGG | 973 |
| | GGAAAGGGAGGAGGAAGA | 427 | GKGGGR | 974 |
| | GGAAAAGGAGGAGTTGGA | 428 | GKGGVG | 975 |
| | GGGAAGGGAGGTGTAGGA | 429 | GKGGVG | 975 |
| | GGAAAGGGGAGAGCAGGT | 430 | GKGRAG | 976 |
| | GGAAAAGGGAGGACTAGG | 431 | GKGRTR | 977 |
| | GGGAAAGGGAGCAGCAGG | 432 | GKGSSR | 978 |
| | GGAAAGGGAGTAGTAAGT | 433 | GKGVVS | 979 |
| | GGAAAGAGAGGAGGAGGG | 434 | GKRGGG | 980 |
| | GGGTTGTGTGGAGTAGGG | 435 | GLCGVG | 981 |
| | GGACTAGGAGCAGTAGGC | 436 | GLGAVG | 982 |
| | GGACTAGGAGCAGTAGGT | 437 | GLGAVG | 982 |
| | GGACTGGGAGCTGTAGGC | 438 | GLGAVG | 982 |
| | GGATTGGGAGGAGTTGCC | 439 | GLGGVA | 983 |
| | GGACTTGGAGGAGTAGGA | 440 | GLGGVG | 984 |
| | GGACTTGGAGGAGTAGGG | 441 | GLGGVG | 984 |
| | GGATTGGGAGGAGTGCGC | 442 | GLGGVR | 985 |
| | GGGTTGAGTGGAGTAGGG | 443 | GLSGVG | 986 |
| | GGAATGGCTGGAGGAGGG | 444 | GMAGGG | 987 |
| | GGTATGTGTGGAGTCGGG | 445 | GMCGVG | 988 |
| | GGAATGGATGGAGAAGGT | 446 | GMDGEG | 989 |
| | GGAATGGAAGCAGCAGGC | 447 | GMEAAG | 990 |
| | GGAATGGAAGGAGAAGGG | 448 | GMEGEG | 991 |
| | GGAATGGAAGGAGAGGGT | 449 | GMEGEG | 991 |
| | GGAATGGAAGGAGTGGGC | 450 | GMEGVG | 992 |
| | GGGATGGAGAGGAGTAGG | 451 | GMERSR | 993 |
| | GGAATGGAAAGAGTAAGG | 452 | GMERVR | 994 |
| | GGAATGGGAGCAGTTGCC | 453 | GMGAVA | 995 |
| | GGAATGGGAGCTGTTGGC | 454 | GMGAVG | 996 |
| | GGAATGGGAGCTGTTGGT | 455 | GMGAVG | 996 |
| | GGAATGGGAGCAGTACTA | 456 | GMGAVL | 997 |
| | GGAATGGGAGATGTTGGC | 457 | GMGDVG | 998 |
| | GGAATGGGAGAAGAAGTA | 458 | GMGEEV | 999 |
| | GGAATGGGAGAATTTGGA | 459 | GMGEFG | 1000 |
| | GGAATGGGAGAAATGGGA | 460 | GMGEMG | 1001 |
| | GGGATGGGAGAAGTTGGT | 461 | GMGEVG | 1002 |
| | GGCATGGGAGAAGTAGTT | 462 | GMGEVV | 1003 |
| | GGAATGGGAGGTGCAGAT | 463 | GMGGAD | 1004 |
| | GGAATGGGGGGAGCACGA | 464 | GMGGAR | 1005 |
| | GGAATGGGGGGAGCATGG | 465 | GMGGAW | 1006 |
| | GGAATGGGAGGAGAGGCT | 466 | GMGGEA | 1007 |
| | GGAATGGGCGGAGAAGCA | 467 | GMGGEA | 1007 |
| | GGAATGGGAGGTGAGGGT | 468 | GMGGEG | 1008 |
| | GGAATGGGAGGAGAAAAA | 469 | GMGGEK | 1009 |
| | GGAATGGGAGGATTTGTA | 470 | GMGGFV | 1010 |
| | GGAATGGGAGGAGGTGGT | 471 | GMGGGG | 1011 |
| | GGAATGGGAGGTGGTGGT | 472 | GMGGGG | 1011 |
| | GGGATGGGAGGAGGTGGT | 473 | GMGGGG | 1011 |
| | GGTATGGGTGGAGGAGGA | 474 | GMGGGG | 1011 |
| | GGAATGGGAGGTGGAGTT | 475 | GMGGGV | 1012 |
| | GGAATGGGAGGCATAGGG | 476 | GMGGIG | 1013 |
| | GGAATGGGAGGCATAGGT | 477 | GMGGIG | 1013 |
| | GGAATGGGAGGCATCGGA | 478 | GMGGIG | 1013 |
| | GGAATGGGAGGGATTGGA | 479 | GMGGIG | 1013 |
| | GGAATGGGCGGGATAGGT | 480 | GMGGIG | 1013 |
| | GGAATGGGTGGAATAGGG | 481 | GMGGIG | 1013 |
| | GGAATGGGTGGCATAGGT | 482 | GMGGIG | 1013 |
| | GGAATGGGTGGTATAGGA | 483 | GMGGIG | 1013 |
| | GGAATGGGTGGTATAGGC | 484 | GMGGIG | 1013 |
| | GGAATGGGTGGTATAGGT | 485 | GMGGIG | 1013 |
| | GGCATGGGTGGAATAGGC | 486 | GMGGIG | 1013 |
| | GGCATGGGTGGTATAGGG | 487 | GMGGIG | 1013 |
| | GGGATGGGAGGAATAGGA | 488 | GMGGIG | 1013 |
| | GGGATGGGAGGGATAGGA | 489 | GMGGIG | 1013 |
| | GGGATGGGAGGTATAGGG | 490 | GMGGIG | 1013 |
| | GGGATGGGTGGAATAGGT | 491 | GMGGIG | 1013 |
| | GGAATGGGAGGACTGGGG | 492 | GMGGLG | 1014 |
| | GGAATGGGAGGATTGGGG | 493 | GMGGLG | 1014 |
| | GGAATGGGAGGCTTGGGA | 494 | GMGGLG | 1014 |
| | GGAATGGGAGGCTTGGGG | 495 | GMGGLG | 1014 |
| | GGAATGGGAGGGTTAGGG | 496 | GMGGLG | 1014 |
| | GGAATGGGAGGGTTGGGG | 497 | GMGGLG | 1014 |
| | GGAATGGGCGGACTAGGA | 498 | GMGGLG | 1014 |
| | GGAATGGGGGGACTAGGG | 499 | GMGGLG | 1014 |
| | GGAATGGGGGGACTCGGG | 500 | GMGGLG | 1014 |
| | GGAATGGGGGGCTTAGGT | 501 | GMGGLG | 1014 |
| | GGAATGGGTGGCTTAGGT | 502 | GMGGLG | 1014 |
| | GGAATGGGTGGTTTAGGA | 503 | GMGGLG | 1014 |
| | GGTATGGGTGGATTAGGT | 504 | GMGGLG | 1014 |
| | GGAATGGGAGGAACAGTT | 505 | GMGGTV | 1015 |
| | GGAATGGGAGGAGTCGGT | 506 | GMGGVG | 1016 |
| | GGAATGGGAGGAGTGGGA | 507 | GMGGVG | 1016 |
| | GGAATGGGAGGAGTTGGT | 508 | GMGGVG | 1016 |
| | GGAATGGGAGGGGTGGGT | 509 | GMGGVG | 1016 |
| | GGAATGGGAGGTGTGGGA | 510 | GMGGVG | 1016 |
| | GGAATGGGCGGGGTTGGT | 511 | GMGGVG | 1016 |
| | GGAATGGGGGGAGTCGGG | 512 | GMGGVG | 1016 |
| | GGAATGGGGGGAGTCGGT | 513 | GMGGVG | 1016 |
| | GGAATGGGGGGAGTTGGT | 514 | GMGGVG | 1016 |
| | GGAATGGGGGGTGTCGGA | 515 | GMGGVG | 1016 |
| | GGAATGGGGGGTGTGGGA | 516 | GMGGVG | 1016 |
| | GGAATGGGTGGAGTTGGC | 517 | GMGGVG | 1016 |
| | GGAATGGGTGGTGTGGGA | 518 | GMGGVG | 1016 |
| | GGAATGGGTGGTGTTGGG | 519 | GMGGVG | 1016 |
| | GGCATGGGAGGAGTGGGT | 520 | GMGGVG | 1016 |
| | GGCATGGGAGGGGTGGGC | 521 | GMGGVG | 1016 |
| | GGCATGGGAGGGGTGGGT | 522 | GMGGVG | 1016 |
| | GGCATGGGAGGGGTTGGT | 523 | GMGGVG | 1016 |
| | GGCATGGGCGGAGTGGGT | 524 | GMGGVG | 1016 |
| | GGCATGGGGGGAGTTGGT | 525 | GMGGVG | 1016 |
| | GGGATGGGAGGAGTTGGG | 526 | GMGGVG | 1016 |
| | GGGATGGGAGGAGTTGGT | 527 | GMGGVG | 1016 |
| | GGGATGGGAGGGGTCGGT | 528 | GMGGVG | 1016 |
| | GGGATGGGAGGTGTGGGT | 529 | GMGGVG | 1016 |
| | GGGATGGGCGGAGTGGGA | 530 | GMGGVG | 1016 |
| | GGGATGGGCGGAGTGGGT | 531 | GMGGVG | 1016 |
| | GGGATGGGGGGAGTTGGT | 532 | GMGGVG | 1016 |
| | GGGATGGGTGGAGTGGGA | 533 | GMGGVG | 1016 |
| | GGGATGGGTGGTGTGGGG | 534 | GMGGVG | 1016 |
| | GGTATGGGAGGGGTTGGT | 535 | GMGGVG | 1016 |
| | GGTATGGGTGGAGTTGGG | 536 | GMGGVG | 1016 |
| | GGAATGGGAAGAGGATGC | 537 | GMGRGC | 1017 |
| | GGAATGGGAGTAGAAGAC | 538 | GMGVED | 1018 |
| | GGAATGGGAGTAGTGGGT | 539 | GMGVVG | 1019 |
| | GGAATGATAGGAGGAGGA | 540 | GMIGGG | 1020 |
| | GGGATGCCAGGAATAGGA | 541 | GMPGIG | 1021 |
| | GGAATGCGAGCAGTAGAG | 542 | GMRAVE | 1022 |
| | GGCATGAGAGGAGCAAGG | 543 | GMRGAR | 1023 |
| | GGAATGAGAGGAAAAGGG | 544 | GMRGKG | 1024 |
| | GGAATGAGAGGACTTGGT | 545 | GMRGLG | 1025 |
| | GGGATGAGAGGAGTAGGC | 546 | GMRGVG | 1026 |
| | GGAATGAGAGGAGTGCGG | 547 | GMRGVR | 1027 |
| | GGAATGGTAGCAATAGGA | 548 | GMVAIG | 1028 |
| | GGAATGGTGGGAGAAGGA | 549 | GMVGEG | 1029 |
| | GGGAATGCGATGAGAAGG | 550 | GNAMRR | 1030 |
| | GGGAATGACAGGATTAGG | 551 | GNDRIR | 1031 |
| | GGGAATGAGATGAGAAGG | 552 | GNEMRR | 1032 |
| | GGGAATGAGAGGAATGGG | 553 | GNERNG | 1033 |
| | GGAAATGAGAGGAGTAAG | 554 | GNERSK | 1034 |
| | GGAAATGGAGGAGCAGGA | 555 | GNGGAG | 1035 |
| | GGAAATGGAGGAATGGGG | 556 | GNGGMG | 1036 |
| | GGGAATGGGATTAGAAGG | 557 | GNGIRR | 1037 |
| | GGGAATGGGAGGAATGTG | 558 | GNGRNV | 1038 |
| | GGGAATGGAAGGAGAAGG | 559 | GNGRRR | 1039 |
| | GGGAATGGAAGGAGCAAG | 560 | GNGRSK | 1040 |
| | GGTAATGGAAGGAGTTGG | 561 | GNGRSW | 1041 |
| | GGGAATGGGAGTAATGGG | 562 | GNGSNG | 1042 |
| | GGGAATCGGAGGAGTATT | 563 | GNRRSI | 1043 |
| | GGGAATGTGAGCAGTAGC | 564 | GNVSSS | 1044 |
| | GGAAATTGGAGGAGCAGG | 565 | GNWRSR | 1045 |
| | GGGCAGGGGAGGGGTAGG | 566 | GQGRGR | 1046 |
| | GGAAGGGGAGAAGGAGGT | 567 | GRGEGG | 1047 |
| | GGAAGGGGAGGAGTGGAA | 568 | GRGGVE | 1048 |
| | GGAAGGGGTGGTGTAGGG | 569 | GRGGVG | 1049 |
| | GGAAGGGGAAGAGAAGGA | 570 | GRGREG | 1050 |
| | GGAAGCGGAGGAGGAGGA | 571 | GSGGGG | 1051 |
| | GGAAGTGGAGGAGGAGGC | 572 | GSGGGG | 1051 |
| | GGGAGTGGAAGGAGGAGG | 573 | GSGRRR | 1052 |
| | GGGAGTGGGAGCAGTTGG | 574 | GSGSSW | 1053 |
| | GGGAGTGGGAGTAGTTGG | 575 | GSGSSW | 1053 |
| | GGAACTGGAGGAGGAGGC | 576 | GTGGGG | 1054 |
| | GGGACTGGAGGAGTAGTG | 577 | GTGGVV | 1055 |
| | GGGACTGTGAAGAGTAGG | 578 | GTVKSR | 1056 |
| | GGAGTAGGAGGAGGAGGA | 579 | GVGGGG | 1057 |
| | GGAGTGGGAGGTGGAGGT | 580 | GVGGGG | 1057 |
| | CATGAAAGGGAGGAGTATGCA | 581 | HEREEYA | 1058 |
| | ATTGAAAGGGAGGAGTTGATA | 582 | IEREELI | 1059 |
| | AAGGATGCGAGGAGTAGG | 583 | KDARSR | 1060 |
| | AAGGAATGTGAGGAGAAGTAT | 584 | KECEEKY | 1061 |
| | AAGGAAGGCGAGGAGGAGGAG | 585 | KEGEEEE | 1062 |
| | AAGGAAGGGGAAGAGAAGGAG | 586 | KEGEEKE | 1063 |
| | AAGGAAGGGGAGAAGGAGGTG | 587 | KEGEKEV | 1064 |
| | AAGGAATTGGAGGAGTACCAC | 588 | KELEEYH | 1065 |
| | AAGGAATGGGGGGAGCATGGA | 589 | KEWGEHG | 1066 |
| | AAGCATGCGAGGAGTAGG | 590 | KHARSR | 1067 |
| | AAGAAAGGGAAGAGTAGG | 591 | KKGKSR | 1068 |
| | AAAATGGGAGAGGTAGGC | 592 | KMGEVG | 1069 |
| | AAGAATGAGAGGATTCGG | 593 | KNERIR | 1070 |
| | AAGAATGGGAGAAGTAGG | 594 | KNGRSR | 1071 |
| | AAGGTATGGGAGGAGGATGCT | 595 | KVWEEDA | 1072 |
| | CTGGCAATGGAGGAGGAGGAA | 596 | LAMEEEE | 1073 |
| | TTGGATGGGGAGGAGTGGGCT | 597 | LDGEEWA | 1074 |
| | TTGGACAGGGAGGAGAAGGTG | 598 | LDREEKV | 1075 |
| | TTGGAATGCGAGAAGAAGGCA | 599 | LECEKKA | 1076 |
| | CTGGAATTGGAGGATGAGGTT | 600 | LELEDEV | 1077 |
| | TTGGAAAGGGAGGATTTGGAC | 601 | LEREDLD | 1078 |
| | TTGGAAAGGGAAGAGAAGGAG | 602 | LEREEKE | 1079 |
| | TTGGAAAGGGTGGAGAAGGAT | 603 | LERVEKD | 1080 |
| | TTGGAATGGGAGGAGGCAGGG | 604 | LEWEEAG | 1081 |
| | TTGGAGTGGGAGGAAAAGGTA | 605 | LEWEEKV | 1082 |
| | TTAGAATGGGAGAAGAAGGAG | 606 | LEWEKKE | 1083 |
| | TTAGAATGGGAGAAGAAGGTA | 607 | LEWEKKV | 1084 |
| | TTAGAATGGGAGAAGAAGGTG | 608 | LEWEKKV | 1084 |
| | TTGGAATGGGAGAAAAAGGTG | 609 | LEWEKKV | 1084 |
| | TTGGAGTGGGAGAAAAAGGTG | 610 | LEWEKKV | 1084 |
| | TTGGGATGGCACGAGCAGGTT | 611 | LGWHEQV | 1085 |
| | TTGAAATTGGAGGAGTATGAC | 612 | LKLEEYD | 1086 |
| | ATGGACTGGGAGGAGTATGTT | 613 | MDWEEYV | 1087 |
| | ATGGAATGTGAGGATTCGGAG | 614 | MECEDSE | 1088 |
| | ATGGAATGTGAGGAAGAGAGG | 615 | MECEEER | 1089 |
| | ATGGAGGAGGAGGAGGAGGAT | 616 | MEEEEED | 1090 |
| | ATGGAAGGGGCGGAGAAGGAG | 617 | MEGAEKE | 1091 |
| | ATGGGATTGGTGGAGTTGGGA | 618 | MGLVELG | 1092 |
| | ATGCAATGGGAGGTGTTGGAG | 619 | MQWEVLE | 1093 |
| | CAGGAATTGGATGAGTATGAT | 620 | QELDEYD | 1094 |
| | CAGGAATTGGAGGAGCAGAAA | 621 | QELEEQK | 1095 |
| | CAGGAATTGAAGGAGAAGGCT | 622 | QELKEKA | 1096 |
| | CAGGAGTGGGAAGAGTACGTA | 623 | QEWEEYV | 1097 |
| | CAGAAGGGGAGGAGTGGG | 624 | QKGRSG | 1098 |
| | CAGAAATGGAAGGAGTATGGC | 625 | QKWKEYG | 1099 |
| | CAGAAATGGCAGGAGTATGGC | 626 | QKWQEYG | 1100 |
| | CAAATGAGAGGAGTAGGG | 627 | QMRGVG | 1101 |
| | CAAATGAGAGGAGTAGGT | 628 | QMRGVG | 1101 |
| | CGTGATTTGGAGGAGGAGGAT | 629 | RDLEEED | 1102 |
| | AGGGATTGGGAGGAGTTGCCG | 630 | RDWEELP | 1103 |
| | AGGGAAAAGGAGGAGAAGGTA | 631 | REKEEKV | 1104 |
| | AGGGAAAGGGAGCAGCAGGAA | 632 | REREQQE | 1105 |
| | AGGGAGTGGGAGGAGGAGGAA | 633 | REWEEEE | 1106 |
| | CGGGAGTGGGAAGAGTTGGCC | 634 | REWEELA | 1107 |
| | AGGGAATGGGAGGAACAGTTA | 635 | REWEEQL | 1108 |
| | AGGGAATGGGAGAAATGGGAA | 636 | REWEKWE | 1109 |
| | AGGGAATGGGAGGTTAAGGTT | 637 | REWEVKV | 1110 |
| | AGGGAATGGAAGGAGAAGGGT | 638 | REWKEKG | 1111 |
| | AGGGAATGGAAGGAGAGGGTT | 639 | REWKERV | 1112 |
| | AGGATTGGGATGAGGAGG | 640 | RIGMRR | 1113 |
| | AGAAAGGGAGGAGTAGCT | 641 | RKGGVA | 1114 |
| | AGGAAGGGGAGGAGTGGA | 642 | RKGRSG | 1115 |
| | AGGAAATTGGAGGAGCAGGCA | 643 | RKLEEQA | 1116 |
| | CGGAAGCTGAGGAGTAGG | 644 | RKLRSR | 1117 |
| | AGGAAAAGGAGGAGGAGG | 645 | RKRRRR | 1118 |
| | AGGAAACGGAGGAGGAGG | 646 | RKRRRR | 1118 |
| | AGAATGGGAGCAGAAGGT | 647 | RMGAEG | 1119 |
| | CGAATGGGAGGAGCAGCT | 648 | RMGGAA | 1120 |
| | AGAATGGGAGGAGAAGAT | 649 | RMGGED | 1121 |
| | AGAATGGGAGGAGGTGGT | 650 | RMGGGG | 1122 |
| | CGAATGAGAGGAGAAGGG | 651 | RMRGEG | 1123 |
| | AGGAATGAAAGGAGGAGG | 652 | RNERRR | 1124 |
| | AGAAATGAGAGGAGTAAG | 653 | RNERSK | 1125 |
| | AGGAATGGGTGCAGTGGG | 654 | RNGCSG | 1126 |
| | AGGAATGGGAAGATAAGG | 655 | RNGKIR | 1127 |
| | AGGAATGGGAAGAATAAG | 656 | RNGKNK | 1128 |
| | AGGAATGGGATGAAGAGG | 657 | RNGMKR | 1129 |
| | CGCAATGGGAGGGCTAGG | 658 | RNGRAR | 1130 |
| | CGGAATGGGAGAGGTAAG | 659 | RNGRGK | 1131 |
| | AGGAATGGGAGGATTAGA | 660 | RNGRIR | 1132 |
| | AGGAATGGGAGGCTTGGG | 661 | RNGRLG | 1133 |
| | CGGAATGGGAGGCTTGGG | 662 | RNGRLG | 1133 |
| | AGGAATGGGAGGAGAAAC | 663 | RNGRRN | 1134 |
| | AGAAATGGTAGAAGTAGG | 664 | RNGRSR | 1135 |
| | AGAAATGGGAGGAGCAGC | 665 | RNGRSS | 1136 |
| | AGGAATGGAAGGAGTGTG | 666 | RNGRSV | 1137 |
| | CGGAATGGAAGCAGCAGG | 667 | RNGSSR | 1138 |
| | AGGAATGGGACATGTAGG | 668 | RNGTCR | 1139 |
| | AGGAATGGCTGGAGGAGG | 669 | RNGWRR | 1140 |
| | CGGAATCGGATGAGTCGG | 670 | RNRMSR | 1141 |
| | CGGAATCGTAGGAGTGGG | 671 | RNRRSG | 1142 |
| | AGGAATAGGCGGAGTAGG | 672 | RNRRSR | 1143 |
| | AGGAATGTGAGAAGCAGG | 673 | RNVRSR | 1144 |
| | AGGAATTGGAGTCGTAGG | 674 | RNWSRR | 1145 |
| | AGAAGGGGAGGAGTGGGC | 675 | RRGGVG | 1146 |
| | AGGAGTAGGAGGAGGAGG | 676 | RSRRRR | 1147 |
| | CGGACTGGGAAGAGTACG | 677 | RTGKST | 1148 |
| | CGGACTGGGAGCTGTAGG | 678 | RTGSCR | 1149 |
| | CGGACTCGGAGGAGTTGG | 679 | RTRRSW | 1150 |
| | AGGACTTGGAGGAGTAGG | 680 | RTWRSR | 1151 |
| | AGGTATGGGAGGATTAGT | 681 | RYGRIS | 1152 |
| | CGGTATGGGTGGAGGAGG | 682 | RYGWRR | 1153 |
| | AGTGAATGGGAGGAGGATGAT | 683 | SEWEEDD | 1154 |
| | TCGGAATGGAAGCAGCAGGCA | 684 | SEWKQQA | 1155 |
| | TCGAAGGGAAGGAGTAGG | 685 | SKGRSR | 1156 |
| | AGCATGGGAGGAGGAGGA | 686 | SMGGGG | 1157 |
| | AGCAATGGAAGGAGTAGA | 687 | SNGRSR | 1158 |
| | AGTAATGGGAGGTATAGG | 688 | SNGRYR | 1159 |
| | AGCAATGGGAGCAGGAGG | 689 | SNGSRR | 1160 |
| | ACAGAATGGGAAGACTATGGT | 690 | TEWEDYG | 1161 |
| | ACGGAATGGAAGGAGAAGGGT | 691 | TEWKEKG | 1162 |
| | GTGGAATTGGAGGACATGGTC | 692 | VELEDMV | 1163 |
| | GTGGAACTGGAGGAGAAGGGC | 693 | VELEEKG | 1164 |
| | GTGGAATCGGAGGAGATGGTG | 694 | VESEEMV | 1165 |
| | GTGGAGTGGGAGGAGTTGATG | 695 | VEWEELM | 1166 |
| | GTGGAATGGGAGGTGCAGATT | 696 | VEWEVQI | 1167 |
| | GTGGAATGGGTGGATTGGGAT | 697 | VEWVDWD | 1168 |
| | GTGATTGGTAGGAGGAGG | 698 | VIGRRR | 1169 |
| | GTGATTGGTAGGAGTAGG | 699 | VIGRSR | 1170 |
| | GTGAAATGGGAGGTGAAGGAT | 700 | VKWEVKD | 1171 |
| | GTATTGGGCGGAGTAGGT | 701 | VLGGVG | 1172 |
| | GTAATGGAAGGAGTAGCT | 702 | VMEGVA | 1173 |
| | GTAATGGAAGGAGTAGGG | 703 | VMEGVG | 1174 |
| | GTAATGGAAGGAGTAGGT | 704 | VMEGVG | 1174 |
| | GTAATGGGAGGAGGAGAC | 705 | VMGGGD | 1175 |
| | GTAATGGGAGGAGTAGCC | 706 | VMGGVA | 1176 |
| | GTAATGGGAGGCGTTGGG | 707 | VMGGVG | 1177 |
| | TGGGATGGGAGGTGTGGG | 708 | WDGRCG | 1178 |
| | TGGGATGGAAGGACTAGG | 709 | WDGRTR | 1179 |
| | TGGGATTGGGAGGAGGAAGAA | 710 | WDWEEEE | 1180 |
| | TGGGAAGAGGAGGAGAAGCAG | 711 | WEEEEKQ | 1181 |
| | TGGGAATCGGAGGAGTATTCC | 712 | WESEEYS | 1182 |
| | TGGGAATGGGTGGACTGGGAG | 713 | WEWVDWE | 1183 |
| | TGGAATGCGATGATTAGG | 714 | WNAMIR | 1184 |
| | TGGAATGACAGGAATAGG | 715 | WNDRNR | 1185 |
| | TGGAATGGGAAGAGGATG | 716 | WNGKRM | 1186 |
| | TGGAATGGGATGAGTGGC | 717 | WNGMSG | 1187 |
| | TGGAATGGGATGAGCAAG | 718 | WNGMSK | 1188 |
| | TGGAATGGGATGAGTAAA | 719 | WNGMSK | 1188 |
| | TGGAATGGGATGAGCAGG | 720 | WNGMSR | 1189 |
| | TGGAATGGGATGAGTAGG | 721 | WNGMSR | 1189 |
| | TGGAATGGGAGGCATAGG | 722 | WNGRHR | 1190 |
| | TGGAATGGAAGGAGTGGG | 723 | WNGRSG | 1191 |
| | TGGAATAGGAGGAGAAGA | 724 | WNRRRR | 1192 |
| | TGGAATTGGTGGAGTTGG | 725 | WNWWSW | 1193 |
| | TnGGAGTGGGAGGAAAAGGTA | 726 | XEWEEKV | 1194 |
| PtMIR 180 | TTGTACTTTGTCTTTGTGTTTGAT | 727 | LYFVFVFD | 1195 |
| | AGGTCCTTTGAGTTTATGGTAGAC | 728 | RSFEFMVD | 1196 |
| PtMIR 181 | GCTGCAGTTTGCCTTCTGGTA | 729 | AAVCLLV | 1197 |
| | GCTGCAGTACAGCTTCTGGAT | 730 | AAVQLLD | 1198 |
| | GCAGCAGTAAGGTTTCTGAnn | 731 | AAVRFLX | 1199 |
| | GCTGCAGTTTGGTTTGTGATA | 732 | AAVWFVI | 1200 |
| | GCTGCTGTATGGCTTATGTTG | 733 | AAVWLML | 1201 |
| | GCAGCAGTATGGGTTTTGATA | 734 | AAVWVLI | 1202 |
| | GCTGCAGTATGGGTGCCGATG | 735 | AAVWVPM | 1203 |
| | GCTGGAGTATGGAATCTGAGA | 736 | AGVWNLR | 1204 |
| | TTTGCAGTAGGGCTTGTGAAC | 737 | FAVGLVN | 1205 |
| | TTTTGCAGTAATGCTTCTGAG | 738 | FCSNASE | 1206 |
| | GGCTGCAGTATGGTTACCGAA | 739 | GCSMVTE | 1207 |
| | GGCAGCAATATTGCTTCTGAA | 740 | GSNIASE | 1208 |
| | CACTTCATGATGGCTTCTGAT | 741 | HFMMASD | 1209 |
| | ATATGCAGGATGGCTTCTGTA | 742 | ICRMASV | 1210 |
| | CTGGAGTATGGCATCTGC | 743 | LEYGIC | 1211 |
| | CTCTGGAATACGGCTTCTGAA | 744 | LWNTASE | 1212 |
| | ATGGAGTATGGCTTCGGA | 745 | MEYGFG | 1213 |
| | ATGCAGAATGGCTTCTGG | 746 | MQNGFW | 1214 |
| | AACAGCAATATGGATTCTGAT | 747 | NSNMDSD | 1215 |
| | AATAGCAGTGTGGCTTCTGAG | 748 | NSSVASE | 1216 |
| | AACTGGAGGATGGCTTCAGAT | 749 | NWRMASD | 1217 |
| | CCTGCAGGATTTCTTCTGATT | 750 | PAGFLLI | 1218 |
| | CCAGCAGTCTGCCTTCTGACA | 751 | PAVCLLT | 1219 |
| | CCTGCAGTTTGTCTGCTGACT | 752 | PAVCLLT | 1219 |
| | CCGTGCAATATAGCTTCTGAC | 753 | PCNIASD | 1220 |
| | CCTAAAGAATGGCTTCTGAAG | 754 | PKEWLLK | 1221 |
| | CAGTACGGTATGGCTTCTGAG | 755 | QYGMASE | 1222 |
| | CGCTGCCGTAGTGCTTCTGAT | 756 | RCRSASD | 1223 |
| | TCTGCATTAGGGCTTCTGTTG | 757 | SALGLLL | 1224 |
| | TCGTGCAATATAGCTTCTGAC | 758 | SCNIASD | 1225 |
| | TCATGCAATATCGCTTCTGAA | 759 | SCNIASE | 1226 |
| | TCGTGCAATATAGCTTCTGAG | 760 | SCNIASE | 1226 |
| | TCATGCAATATGGCTTCTGAA | 761 | SCNMASE | 1227 |
| | TCATGCAATGTGGCTTCTGAA | 762 | SCNVASE | 1228 |
| | TCCTGCAGTAAGGGCTCTGAG | 763 | SCSKGSE | 1229 |
| | AGCAGCAGTAAGGTTTCTGAA | 764 | SSSKVSE | 1230 |
| | AGCAGCAGTAAGGTTTCTGAn | 765 | SSSKVSX | 1231 |
| | TCCAGCAGTCTGCCTTCTGAC | 766 | SSSLPSD | 1232 |
| | TCTTCCAGTATGGCTTCTAAA | 767 | SSSMASK | 1233 |
| | AGCTACACAATGGCTTCTGAG | 768 | SYTMASE | 1234 |
| | ACTGCATTGAGGCTTCTGAAT | 769 | TALRLLN | 1235 |
| | ACTGCAGTGTGTATTCTGAAT | 770 | TAVCILN | 1236 |
| | ACTGCAGTAATGCTTCTGGGA | 771 | TAVMLLG | 1237 |
| | ACAGCAGTATGGGTTTTGATA | 772 | TAVWVLI | 1238 |
| | ACTGCAGTATATCTTATGAAC | 773 | TAVYLMN | 1239 |
| | TACTGCAGTATTGCCTCTGAC | 774 | YCSIASD | 1240 |
| | TACTGCAGTATGGTTACCGAA | 775 | YCSMVTE | 1241 |
| | TACTGGAGTATGGCATCTGCA | 776 | YWSMASA | 1242 |
| | TACTGGAGTATGGCATCTGCG | 777 | YWSMASA | 1242 |
| PtMIR 183 | GCGATACTGGAACGGCTCAATCAT | 778 | AILERLNH | 1243 |
| | GGGATATTGGCGCGGCTCAATCAC | 779 | GILARLNH | 1244 |
| | GGGATATTGGCGCGGCTCAATCAA | 780 | GILARLNQ | 1245 |
| | GTGATATTGGAACGGCTCAATCAT | 781 | VILERLNH | 1246 |
| PtMIR184 | GAAGCTCATTTACACTTGGTGGAT | 1376 | EAHLHLVD | 1554 |
| PtMIR185 | ACTTGGGAGCTAACCACACTGCCT | 1377 | TWELTTLP | 1555 |
| | CAAACCAGCTCTCCACACTGCTTC | 1378 | QTSSPHCF | 1556 |
| | CAAGACCAGCAAACCACAGTGTCT | 1379 | QDQQTTVS | 1557 |
| | GAACCAACTAACCAAACTGTCTCG | 1380 | EPTNQTVS | 1558 |
| | GATGATGAGCTAATCACACTGCCT | 1381 | DDELITLP | 1559 |
| | TGGAACCAGCTGACCGAGCTGCCC | 1382 | WNQLTELP | 1560 |
| PtMIR186 | GATGGGAGGAGTAAGAAAGAG | 1383 | DGRSKKE | 1561 |
| | GGAATGGAAGGAGTGGGCAAG | 1384 | GMEGVGK | 1562 |
| | GGAATGGAAGGAGTGGGCAAGACA | 1385 | GMEGVGKT | 1563 |
| | GGAATGGGAGGACTGGGGAAG | 1386 | GMGGLGK | 1564 |
| | GGAATGGGAGGACTGGGGAAGACA | 1387 | GMGGLGKT | 1565 |
| | GGAATGGGAGGAGTCGGTAAA | 1388 | GMGGVGK | 1566 |
| | GGAATGGGAGGAGTCGGTAAAACA | 1389 | GMGGVGKT | 1567 |
| | GGAATGGGAGGAGTGGGAAAA | 1390 | GMGGVGK | 1566 |
| | GGAATGGGAGGAGTGGGAAAAACC | 1391 | GMGGVGKT | 1567 |
| | GGAATGGGAGGAGTTGGTAAA | 1392 | GMGGVGK | 1566 |
| | GGAATGGGAGGAGTTGGTAAAACA | 1393 | GMGGVGKT | 1567 |
| | GGAATGGGAGGAGTTGGTAAAACG | 1394 | GMGGVGKT | 1567 |
| | GGAATGGGAGGATTGGGGAAG | 1395 | GMGGLGK | 1564 |
| | GGAATGGGAGGATTGGGGAAGACT | 1396 | GMGGLGKT | 1565 |
| | GGAATGGGAGGGGTGGGTAAA | 1397 | GMGGVGK | 1566 |
| | GGAATGGGAGGGGTGGGTAAAACC | 1398 | GMGGVGKT | 1567 |
| | GGAATGGGAGGGTTAGGGAAA | 1399 | GMGGLGK | 1564 |
| | GGAATGGGAGGTGTGGGAAAA | 1400 | GMGGVGK | 1566 |
| | GGAATGGGGGGACTAGGGAAA | 1401 | GMGGLGK | 1564 |
| | GGAATGGGGGGAGTCGGGAAG | 1402 | GMGGVGK | 1566 |
| | GGAATGGGGGGAGTCGGGAAGACA | 1403 | GMGGVGKT | 1567 |
| | GGAATGGGGGGAGTCGGTAAA | 1404 | GMGGVGK | 1566 |
| | GGAATGGGGGGAGTTGGTAAA | 1405 | GMGGVGK | 1566 |
| | GGAATGGGTGGAGTTGGCAAA | 1406 | GMGGVGK | 1566 |
| | GGCATGGGAGGAGTGGGTAAA | 1407 | GMGGVGK | 1566 |
| | GGCATGGGAGGGGTGGGCAAA | 1408 | GMGGVGK | 1566 |
| | GGCATGGGAGGGGTGGGTAAA | 1409 | GMGGVGK | 1566 |
| | GGGATGGGAGGAGTTGGGAAG | 1410 | GMGGVGK | 1566 |
| | GGGATGGGAGGAGTTGGGAAGACG | 1411 | GMGGVGKT | 1567 |
| | GGGATGGGAGGAGTTGGTAAA | 1412 | GMGGVGK | 1566 |
| | GGGATGGGAGGGGTCGGTAAA | 1413 | GMGGVGK | 1566 |
| | GGGATGGGAGGTGTGGGTAAA | 1414 | GMGGVGK | 1566 |
| | GGGATGGGCGGAGTGGGTAAG | 1415 | GMGGVGK | 1566 |
| | GGGATGGGCGGAGTGGGTAAGACC | 1416 | GMGGVGKT | 1567 |
| | GGGATGGGCGGAGTGGGTAAGACG | 1417 | GMGGVGKT | 1567 |
| | GGGATGGGGGGAGTTGGTAAA | 1418 | GMGGVGK | 1566 |
| | GGGATGGGGGGTGTGGGCAAA | 1419 | GMGGVGK | 1566 |
| PtMIR241 | ATCAACGCAGCACTAAATGAT | 1420 | INAALND | 1568 |
| | ATCAACGCCGCACTCAATGAC | 1421 | INAALND | 1568 |
| | ATCAACGCCGCACTCAATGAG | 1422 | INAALNE | 1569 |
| | ATCAACGCGGCATTCAATCAC | 1423 | INAAFNH | 1570 |
| | ATCAACGCTGCAAGCAATGGT | 1424 | INAASNG | 1571 |
| | ATCAACGCTGCACTAAATGAA | 1425 | INAALNE | 1569 |
| | ATCAACGCTGCACTCAACGAC | 1426 | INAALND | 1568 |
| | ATCAACGCTGCACTCAATAAC | 1427 | INAALNN | 1572 |
| | ATCAACGCTGCACTCAATAAT | 1428 | INAALNN | 1572 |
| | ATCAACGCTGCCCTCGATAAC | 1429 | INAALDN | 1573 |
| | ATCAACGCTGCTCTCGATAAC | 1430 | INAALDN | 1568 |
| | ATCAATGCAGCACTCAATGAA | 1431 | INAALNE | 1569 |
| | ATCAATGCCGCACTCAATGAC | 1432 | INAALND | 1568 |
| | ATCAATGCTGCACTCAACGAA | 1433 | INAALNE | 1569 |
| | ATCAATGCTGCACTCAACGAT | 1434 | INAALND | 1568 |
| | ATCAATGCTGCACTCAATCAA | 1435 | INAALNQ | 1574 |
| | ATCAATGCTGCACTCAATGAC | 1436 | INAALND | 1568 |
| | ATCAATGCTGCACTCAATGAG | 1437 | INAALNE | 1569 |
| | ATCAATGCTGCACTCAATGAT | 1438 | INAALND | 1568 |
| | ATCAATGCTGCACTTAACGAC | 1439 | INAALND | 1568 |
| | ATCAATGCTGCCCTCAACGAC | 1440 | INAALND | 1568 |
| | ATCAATGCTGCCCTCAATGAC | 1441 | INAALND | 1568 |
| | ATCAATGCTGTACTCTATGGC | 1442 | INAVLYG | 1575 |
| | ATTGACGCTGCACTCAGTAAT | 1443 | IDAALSN | 1576 |
| PtMIR244 | GGGAACATTGACCGATTGTGGGAA | 1444 | GNIDRLWE | 1577 |
| | GGGAACATTGACCGATTGTGGGAA | 1445 | GNIDRLWE | 1577 |
| | GGGATAATGACCGAGTGTGGA | 1446 | GIMTECG | 1578 |
| | GGGATAATGACCGAGTGTGGA | 1447 | GIMTECG | 1578 |
| | TCAAATGTTGACCGAATGTGGACG | 1448 | SNVDRMWT | 1579 |
| | TCAAATGTTGACCGAATGTGGACG | 1449 | SNVDRMWT | 1579 |
| | TCGAACGTCGACCGAATGTGGGAC | 1450 | SNVDRMWD | 1580 |
| | TCGAACGTCGACCGAATGTGGGAC | 1451 | SNVDRMWD | 1580 |
| | TCGAACGTCGATCGAATGTGGGAC | 1452 | SNVDRMWD | 1580 |
| | TCGAACGTCGATCGAATGTGGGAC | 1453 | SNVDRMWD | 1580 |
| | TCGAACGTTGACCGAATGTGGTCA | 1454 | SNVDRMWS | 1581 |
| | TCGAACGTTGACCGAATGTGGTCA | 1455 | SNVDRMWS | 1581 |
| PtMIR244-2 | ATGGGGATAATGACCGAGTGTGGA | 1456 | MGIMTECG | 1582 |
| | CACTCAAATGTTGACCGAATGTGGACG | 1457 | HSNVDRMWT | 1583 |
| | CACTCGAACGTCGACCGAATGTGGGAC | 1458 | HSNVDRMWD | 1584 |
| | CACTCGAACGTCGATCGAATGTGGGAC | 1459 | HSNVDRMWD | 1584 |
| | CACTCGAACGTTGACCGAATGTGGTCA | 1460 | HSNVDRMWS | 1585 |
| | CATGGGAACATTGACCGATTGTGGGAA | 1461 | HGNIDRLWE | 1586 |
| | CTTGTTGAAGATAGACCGGATGTGAAA | 1462 | LVEDRPDVK | 1587 |
| | CTTGTTGAAGATAGACCGGATGTGACA | 1463 | LVEDRPDVT | 1588 |
| PtMIR245 | GTGTTTTTAGACTACGACGGA | 1464 | VFLDYDG | 1589 |
| PtMIR253 | GCTCGAAACCGTGGAGAGAATCGG | 1465 | ARNRGENR | 1590 |
| | GGCTTAGAACTGTGGAAAGAACTG | 1466 | GLELWKEL | 1591 |
| PtMIR255 | CTTTTTGTTGAAGGTCATCTAATG | 1467 | LFVEGHLM | 1592 |
| | CTTTTTGTTGAAGGTCATCTAATG | 1468 | LFVEGHLM | 1592 |
| | CTTTTTGTTGAAGGTCATTTAACG | 1469 | LFVEGHLT | 1593 |
| | CTTTTTGTTGAAGGTCATTTAACG | 1470 | LFVEGHLT | 1593 |
| | GCTTTTGTTGATGGTTCTCTAGTT | 1471 | AFVDGSLV | 1594 |
| | GCTTTTGTTGATGGTTCTCTAGTT | 1472 | AFVDGSLV | 1594 |
| | TATTTCGTTTATGGTCCTCTGAGC | 1473 | YFVYGPLS | 1595 |
| | TATTTCGTTTATGGTCCTCTGAGC | 1474 | YFVYGPLS | 1595 |
| PtMIR257 | TTGAGGAAGAGACTTCAGAAT | 1475 | LRKRLQN | 1596 |
| | TTGAGGGAGAGAGTATCAGAA | 1476 | LRERVSE | 1597 |
| | TTGATGGAGAGAGTTCGGCAG | 1477 | LMERVRQ | 1598 |
| | TTTGAGGGAGAGAGTTCAGTT | 1478 | FEGESSV | 1599 |
| PtMIR274 | ATTGGTATGAAGCCTGGTCCGGAT | 1479 | IGMKPGPD | 1600 |
| | ATTGGTATGAAGCCTGGTCCGGAT | 1480 | IGMKPGPD | 1600 |
| | CCTGGAATGAAGCCTGGTCCGGAT | 1481 | PGMKPGPD | 1601 |
| | CCTGGAATGAAGCCTGGTCCGGAT | 1482 | PGMKPGPD | 1601 |
| | CCTGGGATGAAGCCTGGTCCGGAT | 1483 | PGMKPGPD | 1601 |
| | CCTGGGATGAAGCCTGGTCCGGAT | 1484 | PGMKPGPD | 1601 |
| | GCGGGAGTGAAGTTTGATCCGACG | 1485 | AGVKFDPT | 1602 |
| | GCGGGAGTGAAGTTTGATCCGACG | 1486 | AGVKFDPT | 1602 |
| PtMIR275 | ATGGATGATGTTGGTAGCTTCAAA | 1487 | MDDVGSFK | 1603 |
| | CATAGATCAGGCTGGCAGCTTGTA | 1488 | HRSGWQLV | 1604 |
| | CTAGATTATGCTGGCATCTCCCTT | 1489 | LDYAGISL | 1605 |
| | GAGGTTATGCTGACAGCTTCG | 1490 | EVMLTAS | 1606 |
| PtMIR275-1 | ATGGATGATGTTGGTAGCTTCAAA | 1491 | MDDVGSFK | 1607 |
| | CATAGATCAGGCTGGCAGCTTGTA | 1492 | HRSGWQLV | 1608 |
| | GAGGTTATGCTGACAGCTTCG | 1493 | EVMLTAS | 1609 |
| PtMIR275-2 | AAAGATCAGATTGGCAGCTTCTAC | 1494 | KDQIGSFY | 1610 |
| | ATGGATGATGTTGGTAGCTTCAAA | 1495 | MDDVGSFK | 1607 |
| | CAGAGATCAGGCTGGCAGCTTGTA | 1496 | QRSGWQLV | 1611 |
| | CTGAGATCAGGCTGGCAGCTTGTA | 1497 | LRSGWQLV | 1612 |
| | GAGGTTATGCTGACAGCTTCG | 1498 | EVMLTAS | 1609 |
| PtMIR277 | AAGGTGAAGGAAGCTGTGGAA | 1499 | KVKEAVE | 1613 |
| | AAGGTGAAGGAAGCTGTGGAA | 1500 | KVKEAVE | 1613 |
| | AGATTGAGAAAGTTGTGGAAA | 1501 | RLRKLWK | 1614 |
| | AGATTGAGAAAGTTGTGGAAA | 1502 | RLRKLWK | 1614 |
| | CAGTTCAAGAAAGCTTTGAAG | 1503 | QFKKALK | 1615 |
| | CAGTTCAAGAAAGCTTTGAAG | 1504 | QFKKALK | 1615 |
| PtMIR277-3 | AATCGTTCAAGAAAGCCTGTGGAA | 1505 | NRSRKPVE | 1616 |
| | AATGTTCCAGAGAGCTGTGGATGC | 1506 | NVPESCGC | 1617 |
| | ATTGTTCAGAAAGGCTGTGGGAAA | 1507 | IVQKGCGK | 1618 |
| | CATCGTTCAAGAAAGCCTGTGGAA | 1508 | HRSRKPVE | 1619 |
| | CTGTTCGGGAAAGTGGTGGAA | 1509 | LFGKVVE | 1620 |
| | CTTTTCAAGAAAGCTGAGGAG | 1510 | LFKKAEE | 1621 |
| | GGGTGTTCAAGTGGGTTGTGGAAT | 1511 | GCSSGLWN | 1622 |
| | GTGTTTAAGGAAGTTGTGGCA | 1512 | VFKEVVA | 1623 |
| | TATTATTCAAGAAAGTTGTGGGAG | 1513 | YYSRKLWE | 1624 |
| | TTCTTGAAGAAAGCTGTGGAG | 1514 | FLKKAVE | 1625 |
| PtMIR282 | AAAGGTGCAGGTGCAGATGTAATA | 1515 | KGAGADVI | 1626 |
| | AAAGGTGCAGGTGCAGATTTA | 1516 | KGAGADL | 1627 |
| | GAAGGTGCAGATGCAGATGAA | 1517 | EGADADE | 1628 |
| | TGGGGTGCGGGTGCTAATGCA | 1518 | WGAGANA | 1629 |
| PtMIR284 | GGCTATATCTCTCCTGAGCTT | 1519 | GYISPEL | 1630 |
| | GGCTCTATACCTCCTGAGCTT | 1520 | GSIPPEL | 1631 |
| | GGGGCTATCCCTCCTGGACTT | 1521 | GAIPPGL | 1632 |
| | GGTGCTAACCCTCCTGAGCCT | 1522 | GANPPEP | 1633 |
| | GGTGCTGTCCCTGCTGGGCTT | 1523 | GAVPAGL | 1634 |
| | GGTGTTGTCCCACCTGAGCTT | 1524 | GVVPPEL | 1635 |
| | GGTGTTGTCCCGCCTGAGCTT | 1525 | GVVPPEL | 1635 |
| | GTGCTGGCCTTCCTGAGCTTC | 1526 | VLAFLSF | 1636 |
| PtMIR287 | AAAATCAAGGACTTGCAATTCTTT | 1527 | KIKDLQFF | 1637 |
| | AATCAAGGAATGGCAATTCTG | 1528 | NQGMAIL | 1638 |
| | AATGAAGGCACCGCAATTCTA | 1529 | NEGTAIL | 1639 |
| | AATGAAGGCACTGCAATTTTA | 1530 | NEGTAIL | 1639 |
| | AATGAAGGCATTGCAAATCTG | 1531 | NEGIANL | 1640 |
| | CAATCTAGGAATTGCAATTCTCTA | 1532 | QSRNCNSL | 1641 |
| | CATCAAGGGGATGCAATTCTG | 1533 | HQGDAIL | 1642 |
| | GAACAAGGCATTGCAGTTCTT | 1534 | EQGIAVL | 1643 |
| | GAATGGAAGCACTGCAATTCTTCG | 1535 | EWKHCNSS | 1644 |
| | GACCGAGGCACTGCAATTCTA | 1536 | DRGTAIL | 1645 |
| | GACCGAGGCACTGCGATTCTA | 1537 | DRGTAIL | 1645 |
| | GGAATCAAGGCACTGCAATTGCAT | 1538 | GIKALQLH | 1646 |
| PtMIR291 | AGTGATATTGATTGGCTTGTT | 1539 | SDIDWLV | 1647 |
| | AGTGATGTTGATTTTGTTCGT | 1540 | SDVDFVR | 1648 |
| | CGGGTGATATTGGTTCGGCTCAAG | 1541 | RVILVRLK | 1649 |
| PtMIR295 | ACTGCTGTTAATTCATGGGTTACT | 1542 | TAVNSWVT | 1650 |
| PtMIR297 | TTGCAAGGGGAGCCCAACAGC | 1543 | LQGEPNS | 1651 |
| PtMIR298 | CTATGGGAGGCTTTGGAGAGG | 1544 | LWEALER | 1652 |
| | GGGATGGGAGGAGTTGGGAAG | 1545 | GMGGVGK | 1653 |
| | GGTATGGTAGGTCTTGGAAAG | 1546 | GMVGLGK | 1654 |
| | GTATGGGAGGCTTGGAAAGCA | 1547 | VWEAWKA | 1655 |
| PtMIR302 | GTTTTATCTGGGGCACTAGTACTGGGG | 1548 | VLSGALVLG | 1656 |
| PtMIR304 | TGGTGGGCAAGTCGTCCTTGGCTA | 1549 | WWASRPWL | 1657 |
| PtMIR310 | GAGAGTTGTCTTGCGTACACTTTA | 1550 | ESCLAYTL | 1658 |
| PtMIR315 | CTTAATTTGATCGAGTTATTGATG | 1551 | LNLIELLM | 1659 |
| | GCTAATCAGAGCGAGCCATTGAAT | 1552 | ANQSEPLN | 1660 |
| | GCTTACCTGGCCGAGCCGTTGGAC | 1553 | AYLAEPLD | 1661 |

TABLE 4
Comparisons of Pinus taeda and Arabidopsis miRNAs and miRNA Genes

| miRNA | Arabidopsis | | | | gene sequence |
|---|---|---|---|---|---|
| gene family | family name | Expressed | name of miRNA | name of gene | (SEQ ID NO:) |
| LpMIR1 | | N.A. | LpmiR1 | LpMIR1 | — |
| | | | (SEQ ID NO: 1662) | | |
| LpMIR2 | | N.A. | LpmiR2 | LpMIR2 | — |
| | | | (SEQ ID NO: 1663) | | |
| LpMIR7 | similar to AthMIR159 and | N.A. | LpmiR7 | LpMIR7 | — |
| | AthMIR319 | | (SEQ ID NO: 1664) | | |
| | | | LpmiR7-1 | LpMIR7-1 | — |
| | | | (SEQ ID NO: 1665) | | |
| | | | LpmiR7-2 | LpMIR7-2 | — |
| | | | (SEQ ID NO: 1666) | | |
| | | | LpmiR7-3 | LpMIR7-3 | — |
| | | | (SEQ ID NO: 1667) | | |
| | | | LpmiR7-4 | LpMIR7-4 | — |
| | | | (SEQ ID NO: 1668) | | |
| | | | LpmiR7-5 | LpMIR7-5 | — |
| | | | (SEQ ID NO: 1669) | | |
| | | | LpmiR7-6 | LpMIR7-6 | — |
| | | | (SEQ ID NO: 1670) | | |
| | | | LpmiR7-7 | LpMIR7-7 | 1713 |
| | | | (SEQ ID NO: 1671) | | |
| | | | LpmiR7-8 | LpMIR7-8 | 1714 |
| | | | (SEQ ID NO: 1672) | (antisense of | |
| | | | | LpMIR7-4) | |
| | | | LpmiR7-9 | LpMIR7-9 | 1715 |
| | | | (SEQ ID NO: 1673) | | |
| LpMIR9 | AthmiR160 | N.A. | LpmiR9 | LpMIR9 | — |
| | | | (SEQ ID NO: 1674) | | |
| LpMIR178 | similar to AthmiR156 | N.A. | LpmiR178 | LpMIR178 | — |
| | | | (SEQ ID NO: 1675) | | |
| | | | LpmiR178-1 | LpMIR178-1 | 1716 |
| | | | (SEQ ID NO: 1676) | | |
| | | | LpmiR178-2 | LpMIR178-2 | 1717 |
| | | | (SEQ ID NO: 1677) | | |
| LpMIR26 | | N.A. | LpmiR26 | LpMIR26 | — |
| | | | (SEQ ID NO: 1678) | | |
| | | | LpmiR26-1 | LpMIR26-1 | 1718 |
| | | | (SEQ ID NO: 1679) | | |
| | | | LpmiR26-2 | LpMIR26-2 | 1719 |
| | | | (SEQ ID NO: 1680) | | |
| LpMIR27 | | N.A. | LpmiR27 | LpMIR27a | 1720 |
| | | | (SEQ ID NO: 1681) | LpMIR27b | 1721 |
| | | | | LpMIR27c | 1722 |
| LpMIR28 | | N.A. | LpmiR28 | LpMIR28 | 1723 |
| | | | (SEQ ID NO: 1682) | | |
| LpMIR77 | | N.A. | LpmiR77 | LpMIR77 | 1724 |
| | | | (SEQ ID NO: 1683) | | |
| LpMIR82 | | N.A. | LpmiR82 | LpMIR82 | — |
| | | | (SEQ ID NO: 1684) | | |
| | | | LpmiR82-1 | LpMIR82-1 | 1725 |
| | | | (SEQ ID NO: 1685) | | |
| | | | LpmiR82-2 | LpMIR82-2 | 1726 |
| | | | (SEQ ID NO: 1686) | | |
| LpMIR89 | | N.A. | LpmiR89 | LpMIR89 | — |
| | | | (SEQ ID NO: 1687) | | |
| | | | LpmiR89-1 | LpMIR89-1 | 1727 |
| | | | (SEQ ID NO: 1688) | | |
| LpMIR95 | | N.A. | LpmiR95 | LpMIR95a | 1728 |
| | | | (SEQ ID NO: 1689 or | LpMIR95b | 1729 |
| | | | SEQ ID NO: 1690) | | |
| LpMIR100 | | N.A. | LpmiR100 | LpMIR100 | — |
| | | | (SEQ ID NO: 1691) | | |
| | | | LpmiR100-1 | LpMIR100-1a | 1730 |
| | | | (SEQ ID NO: 1692) | LpMIR100-1b | 1731 |
| LpMIR119 | | N.A. | LpmiR119 | LpMIR119a | 1732 |
| | | | (SEQ ID NO: 1693) | LpMIR119b | 1733 |
| LpMIR176 | | N.A. | LpmiR176 | LpMIR176 | — |
| | | | (SEQ ID NO: 1694) | | |
| | | | LpmiR176-1 | LpMIR176-1 | 1734 |
| | | | (SEQ ID NO: 1695) | | |
| | | | LpmiR176-2 | LpMIR176-2a | 1735 |
| | | | (SEQ ID NO: 1696) | LpMIR176-2b | 1736 |
| | | | LpmiR176-3 | LpMIR176-3a | 1737 |
| | | | (SEQ ID NO: 1697) | LpMIR176-3b | 1738 |
| LpMIR170 | | N.A. | LpmiR170 | LpMIR170 | — |
| | | | (SEQ ID NO: 1698 or | | |
| | | | SEQ ID NO: 1699) | | |
| | | | LpmiR170-1 | LpMIR170-1a | 1739 |
| | | | (SEQ ID NO: 1700 or | LpMIR170-1b | 1740 |
| | | | SEQ ID NO: 1701) | | |
| | | | LpmiR170-2 | LpMIR170-2a | 1741 |
| | | | (SEQ ID NO: 1702 or | LpMIR170-2b | 1742 |
| | | | SEQ ID NO: 1703) | | |
| | | | LpmiR170-3 | LpMIR170-3 | 1743 |
| | | | (SEQ ID NO: 1704 or | | |
| | | | SEQ ID NO: 1705) | | |
| LpMIR274 | AthMIR166 | N.A. | LpmiR274 | LpMIR274a | 1744 |
| | | | (SEQ ID NO: 1706) | LpMIR274b | 1745 |
| LpMIR277 | AthMIR396 | N.A. | LpmiR277 | LpMIR277 | 1746 |
| | | | (SEQ ID NO: 1707) | | |
| | | | LpmiR277-1 | LpMIR277-1 | — |
| | | | (SEQ ID NO: 1708) | | |
| LpMIR279 | AthMIR408 | N.A. | LpmiR279 | LpMIR279 | 1747 |
| | | | (SEQ ID NO: 1709 or | | |
| | | | SEQ ID NO: 1710) | | |
| LpMIR472 | | N.A. | LpmiR472 | LpMIR472 | — |
| | | | (SEQ ID NO: 1711) | | |
| | | | LpmiR472-1 | LpMIR472-1 | 1748 |
| | | | (SEQ ID NO: 1712) | | |

TABLE 5

Pinus taeda miRNA Target Sequences

| miRNA | | SEQ | Encoded | SEQ |
|---|---|---|---|---|
| gene | | ID | peptide | ID |
| family | Target sequence | NO: | sequence | NO: |
| LpmiR1 | AAAGCTGATTCGCACCAGGTGG | 1749 | n.d. | — |
| LpmiR100 | CGATAAACCATCGTGGAGCAGATG | 1750 | n.d. | — |
| | CGATAAACCATCGTGGAGCAGATG | 1751 | n.d. | — |
| | TCATAAGCCACCGAGGGGCGTATG | 1752 | n.d | — |
| | TTTCATCAACCAACGAGGGCCAAA | 1753 | FHQPTRAK | 1838 |
| LpmiR119 | CCGTGGTCTGGATGTCAAGAACAT | 1754 | PWSGCQEH | 1839 |
| | CGGTGGTCCGGAGGTCAAGAACAT | 1755 | RWSGGQEH | 1840 |
| | CGTGGCCCTGATGTCAAGAACATT | 1756 | RGPDVKNI | 1841 |
| | CGTGGTCTAGATGCCAAGAACATT | 1757 | RGLDAKNI | 1842 |
| | GTGGCCCTGATGTCAAGAACA | 1758 | VALMSRT | 1843 |
| | GTGGTCCAGATGTAAAGAAAA | 1759 | n.d. | — |
| | GTGGTCCGGAGGTCAAGAACA | 1760 | VVRRSRT | 1844 |
| | TCGCGGCCCAGATGTCAAGAACAC | 1761 | SRPRCQEH | 1845 |
| LpmiR176 | CACCAATGGCATTCTTTGATG | 1762 | HQWHSLM | 1846 |
| | CGGCAATGGCATGCCCTGTTT | 1763 | RQWHALF | 1847 |
| | CGTCAATGCTATGCTCTGTTC | 1764 | RQCYALF | 1848 |
| LpmiR178 | GGCCGTGCTCTCTCTCTTCTG | 1765 | GRALSLL | 1849 |
| | GGGCGTGCTCTCTCTCTTCTG | 1766 | GRALSLL | 1849 |
| | GGTGTGCTCTCTCTCTTCTGT | 1767 | GVLSLFC | 1850 |
| | GGTTGTGCTCTCTCTCTTCTG | 1768 | GCALSLL | 1851 |
| | TCTGTGCTTCCTCTCTTCTGA | 1769 | n.d. | — |
| | TGGCTGTGCTCTCTCTCTTCTGTC | 1770 | WLCSLSSV | 1852 |
| LpmiR26 | AAATGTGGATTGGCGAAGGGCTGG | 1771 | KCGLAKGW | 1853 |
| | AATTGTGGATAGGAGAAGGGCTGG | 1772 | n.d. | — |
| | ATCGTGTGGTTGGGAGAAGGGTTG | 1773 | IVWLGEGL | 1854 |
| | ATTGTTGATAGCAGAAGGGTTGAC | 1774 | IVDSRRVD | 1855 |
| | CAGTTGTGGATAGGAGAAGGGCTG | 1775 | QLWIGEGL | 1856 |
| | CTTGTGGATTGGAGAGGGTCTTCT | 1776 | LVDWRGSS | 1857 |
| | GAAATGTGGATAGCGGAGGGGCTG | 1777 | EMWIAEGL | 1858 |
| | TTTGTGGATAGTAGATGGGTGGGC | 1778 | FVDSRWVG | 1859 |
| LpmiR27 | ACTGTTCTGGCGTCCTGTTACTGG | 1779 | TVLASCYW | 1860 |
| | AGCTCCGGCATCTTGGTGCTG | 1780 | SSGILVL | 1861 |
| | ATGCAGTGCATCCTGGTACTG | 1781 | MQCILVL | 1862 |
| | CAGAACTGTTATCCTGGTGCTGGT | 1782 | QNCYPGAG | 1863 |
| | CTCACAGGCGTCCTGGTGCTG | 1783 | LTGVLVL | 1864 |
| | GACATTGGCATCCTGATGCTG | 1784 | DIGILML | 1865 |
| | TGCACTGGTATTCTGTAACTT | 1785 | n.d. | — |
| | TTGCTCTGACATTCTGGTATTGAT | 1786 | n.d. | — |
| LpmiR28 | GAAAAACAGTAGCAGATTCAAATG | 1787 | n.d. | — |
| | GAAACAGAGACAGATTCTGAGTGA | 1788 | n.d. | — |
| | GGAACAGTAATAGATTCTGGCACT | 1789 | GTVIDSGT | 1866 |
| | GTGAAGCAGTAACGGATTCCTATA | 1790 | n.d. | — |
| | TTGATACAGTAACAGATTCCGTTA | 1791 | n.d. | — |
| LpmiR7 | CAGGGAGCTCCCTTCGTTCTGACG | 1792 | QGAPFVLT | 1867 |
| | GGGAGCTTTCTTCAGTCCAAC | 1793 | GSFLQSN | 1868 |
| | GGGTGCTTCCTTCAGGCCAAC | 1794 | GCFLQAN | 1869 |
| | GTTGGAGCTCCCTTCAGTCCAACC | 1795 | VGAPFSPT | 1870 |
| LpmiR7-1 | ACGGGGAGCTTTCTTCAGTCCAAC | 1796 | TGSFLQSN | 1871 |
| | GTTGGAGCTCCCTTCAGTCCAACC | 1797 | VGAPFSPT | 1872 |
| LpmiR7-2 | ATTGGAGCTCCCTTCAAGCCAATC | 1798 | IGAPFKPI | 1873 |
| | GTTGGAGCTCCCTTCAGTCCAACC | 1799 | VGAPFSPT | 1872 |
| | TAGAGCTTTCTTCAGATCGAA | 1800 | n.d. | — |
| | TGGAGCTCCCTTCAAGCCAAT | 1801 | WSSLQAN | 1874 |
| LpmiR7-3 | GGAGCTCCCTTCAGTCCAACC | 1802 | GAPFSPT | 1875 |
| | GGGAGCTTTCTTCAGTCCAAC | 1803 | GSFLQSN | 1876 |
| LpmiR77 | ACCGGATCCCACGAAGCCTGC | 1804 | TGSHEAC | 1877 |
| | CACAGGATCCCACGCAGTTTGATC | 1805 | HRIPRSLI | 1878 |
| | CCGGATCCCACAAAGCCTGAT | 1806 | PDPTKPD | 1879 |
| | CCGGATCCCACACAGCCTGAT | 1807 | PDPTQPD | 1880 |
| | CCGGATCCCACGAAGCCTGCT | 1808 | PDPTKPA | 1881 |
| | GCCGGATCCCACCCAGCTTGC | 1809 | AGSHPAC | 1882 |
| | TACCAGATCCCACACAGCCTGCTT | 1810 | YQIPHSLL | 1883 |
| LpmiR82 | AAGCTGCCAGACTCGCTCGGGACT | 1811 | KLPDSLGT | 1884 |
| | AATCTGCCAGACTCCTTCGGGGAT | 1812 | NLPDSFGD | 1885 |
| | ACGCTGCCAGACTCGCTCGGGACT | 1813 | TLPDSLGT | 1886 |
| | CGCTGCTGGACTCGCTTGGGA | 1814 | RCWTRLG | 1887 |
| | CTCTGCCAGATTCCTTCGGGA | 1815 | LCQIPSG | 1888 |
| | CTTTGCCAGACTCGGTTGGGA | 1816 | LCQTRLG | 1889 |
| | GCTCCCAGACTCGCTTGGGAA | 1817 | APRLAWE | 1890 |
| | GCTGCCAGACTCGCTGGGGAA | 1818 | AARLAGE | 1891 |
| | GCTGCCAGACTCGCTGGGGGA | 1819 | AARLAGG | 1892 |
| | GCTGCCAGACTCGGTTGGGAA | 1820 | AARLGWE | 1893 |
| | GCTTCCAGACTCGTTCGGGAA | 1821 | ASRLVRE | 1894 |
| | TCTCCCAGACTCGGTTGGGAA | 1822 | SPRLGWE | 1895 |
| | TCTGCCAGACTCGCTCGGGAA | 1823 | SARLARE | 1896 |
| | TCTGCCAGACTCGCTGGGGAA | 1824 | SARLAGE | 1897 |
| | TCTGCCAGGCTTGCTTGTGAA | 1825 | SARLACE | 1898 |
| | TTTGCCAGATTCGGTTGGGAA | 1826 | FARFGWE | 1899 |
| | TTTGCCAGATTCGGTTGGGAG | 1827 | FARFGWE | 1899 |
| LpmiR89 | GTCTTATCTTTTACTGGCGGT | 1828 | VLSFTGG | 1900 |
| LpmiR9 | CTGGCATACAGGGGGCCTGGATCA | 1829 | LAYRGPGS | 1901 |
| | GCAGGCATGCAGGGAGCCAGGCAT | 1830 | AGMQGARH | 1902 |
| LpmiR95 | AGAGGCCCATGGGATTCTCTGGAG | 1831 | RGPWDSLE | 1903 |
| | TGGCGCATTGTGTTTTCGGAGAAA | 1832 | WRIVFSEK | 1904 |
| LpmiR95- | ACAGCGAATTAGCTTTCTGGAGAA | 1833 | n.d. | — |
| 1 | AGGGAAATGGATTCCCAGAGA | 1834 | REMDSQR | 1905 |
| | GAGCCGATTGGATTCCTGCAGAAT | 1835 | EPIGFLQN | 1906 |
| | GGTGAATTGGATTCATGGACT | 1836 | GELDSWT | 1907 |
| | GTTGGGAATTGGAATCCCTGAGAT | 1837 | n.d. | — |

n.d.: not determined

Thus, in some embodiments, a plant gene that is targeted for modulation has a nucleic acid sequence comprising any of SEQ ID NOs. 176-781, 1376-1553, and 1749-1837, and encodes a polypeptide having an amino acid sequence comprising any of SEQ ID NOs: 782-1246, 1554-1661, and 1838-1907. Furthermore, based on the knowledge that miRNAs can tolerate mismatches with their targets and still modulate the expression of those targets, in some embodiments a plant gene that is targeted for modulation comprises a nucleic acid sequence at least about 70% identical to any of SEQ ID NOs: 176-781, 1376-1553, and 1749-1837, and encodes a polypeptide comprising an amino acid sequence have 5 or fewer (e.g., 5, 4, 3, 2, or 1) changed amino acids as compared to the amino acids disclosed as SEQ ID NOs: 782-1246, 1554-1661, and 1838-1907.

Using the techniques disclosed in Examples 1-6, additional plant genes can be selected and miRNAs designed to modulate the expression of the genes in any desired plant. Additionally, the basic methodology disclosed in these Examples can be used to isolate miRNAs from any desired plant and to identify genes that can be targeted using the methods disclosed herein.

For example, the techniques disclosed in Examples 1-6 were employed to identify genes from Pinus taeda and to design miRNAs to modulate the expression of genes in Pinus sp. These sequences are summarized in Table 4.

In addition, knowledge of the sequence of a gene and/or a gene product can be used to design miRNAs to target the expression of the gene in any plant. For example, in some embodiments, genes associated with lignin biosynthesis are targeted for modulation. Lignin is a major component of wood, and the regulation of its biosynthesis has can have a major impact on paper and pulping processes. Several genes have been identified that are involved in the biosynthesis of lignin including, but not limited to sinapyl alcohol dehydrogenase (SAD), cinnamyl alcohol dehydrogenase (CAD), 4-coumarate:CoA ligase (4CL), cinnamoyl CoA O-methyltransferase (CCoAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), and phenylalanine ammonia lyase (PAL). Reviewed in Anterola & Lewis, 2002; Boerjan et al., 2003. Reduction in the activities of one or more of these genes has been shown to result in reduced lignin deposition (see Anterola & Lewis, 2002; Boerjan et al., 2003), and thus these genes provide potential targets for miRNA-mediated gene expression modulation.

In some embodiments, genes associated with cellulose biosyntheses are targeted for modulation. Representative, non-limiting genes that have been identified that are associated with cellulose biosynthesis include cellulose synthase (CeS; also referred to as CESA in some plants), cellulose synthase-like (CSL), glucosidase, glucan synthase, Korrigan endocellulase, callose synthase, and sucrose synthase.

In some embodiments, other plant genes are targeted for modulation using miRNAs. A non-limiting list of gene families that can be targeted include hormone-related genes, including but not limited to isopentyl transferase (ipt), gibberellic acid (GA) oxidase, auxin (AUX), auxin-responsive and auxin-induced genes, and members of the rooting locus (ROL) gene family; hemicellulose-related genes, disease-related genes, stress-related genes, growth-related genes and transcription factors.

It is understood that the target genes listed hereinabove are exemplary only, and that the methods and compositions of the presently disclosed subject matter can be applied to modulate the expression of any desired gene in any desired plant.

V. Nucleic Acids

The nucleic acid molecules employed in accordance with the presently disclosed subject matter include any nucleic acid molecule encoding a plant gene product, as well as the nucleic acid molecules that are used in accordance with the presently disclosed subject matter to modulate the expression of a plant gene. Thus, the nucleic acid molecules employed in accordance with the presently disclosed subject matter include, but are not limited to, the nucleic acid molecules described herein (for example, SEQ ID NOs: 1-1907); sequences substantially identical to those described herein (for example, sequences at least 70% identical to any of SEQ ID NOs: 1-1907); and subsequences and elongated sequences thereof. The presently disclosed subject matter also encompasses genes, cDNAs, chimeric genes, and vectors comprising the disclosed nucleic acid sequences.

An exemplary nucleotide sequence employed in the methods disclosed herein comprises sequences that are complementary to each other, the complementary regions being capable of forming a duplex of, in some embodiments, at least about 15 to 300 basepairs, and in some embodiments, at least about 15-24 basepairs. One strand of the duplex comprises a nucleic acid sequence of at least 15 contiguous bases having a nucleic acid sequence of a nucleic acid molecule of the presently disclosed subject matter. In one example, one strand of the duplex comprises a nucleic acid sequence comprising 15, 16, 17, or 18 nucleotides, or even longer where desired, such as 19, 20, 21, 22, 23, 24, 25, 26, 27, 28, 29, or 30 nucleotides, or up to the full length of any of those nucleic acid sequences described herein. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The term “subsequence” refers to a sequence of a nucleic acid molecule or amino acid molecule that comprises a part of a longer nucleic acid or amino acid sequence. An exemplary subsequence is a sequence that comprises part of a duplexed region of a pri-miRNA or a pre-miRNA including, but not limited to the nucleotides that become the mature miRNA after nuclease action or a single-stranded region in an miRNA precursor.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

Nucleic acids of the presently disclosed subject matter can be cloned, synthesized, recombinantly altered, mutagenized, or subjected to combinations of these techniques. Standard recombinant DNA and molecular cloning techniques used to isolate nucleic acids are known in the art. Exemplary, non-limiting methods are described by Silhavy et al., 1984; Ausubel et al., 1989; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

VI. Vectors

In some embodiments of the presently disclosed subject matter, miRNA precursor molecules are expressed from transcription units inserted into nucleic acid vectors (alternatively referred to generally as “recombinant vectors” or “expression vectors”). A vector is used to deliver a nucleic acid molecule encoding an miRNA into a plant cell to target a specific plant gene. The recombinant vectors can be, for example, DNA plasmids or viral vectors. Various expression vectors are known in the art. The selection of the appropriate expression vector can be made on the basis of several factors including, but not limited to the cell type wherein expression is desired. For example, Agrobacterium-based expression vectors can be used to express the nucleic acids of the presently disclosed subject matter when stable expression of the vector insert is sought in a plant cell.

In some embodiments, a vector is also used to deliver a nucleic acid molecule encoding an siRNA into a plant cell to target a specific miRNA precursor.

VI.A. Promoters

The expression of the nucleotide sequence in the expression cassette can be under the control of a constitutive promoter or an inducible promoter that initiates transcription only when the host cell is exposed to some particular external stimulus. For bacterial production of an miRNA and/or an siRNA, exemplary promoters include Simian virus 40 early promoter, a long terminal repeat promoter from retrovirus, an actin promoter, a heat shock promoter, and a metallothionein protein. For in vivo production of an miRNA and/or an siRNA in plants, exemplary constitutive promoters are derived from the CaMV 35S, rice actin, and maize ubiquitin genes, each described herein below. Exemplary inducible promoters for this purpose include the chemically inducible PR-1a promoter and a wound-inducible promoter, also described herein below.

Selected promoters can direct expression in specific cell types (such as leaf epidermal cells, mesophyll cells, root cortex cells) or in specific tissues or organs (roots, leaves or flowers, for example). Exemplary tissue-specific promoters include well-characterized root-, pith-, and leaf-specific promoters, each described herein below.

Depending upon the host cell system utilized, any one of a number of suitable promoters can be used. Promoter selection can be based on expression profile and expression level. The following are non-limiting examples of promoters that can be used in the expression cassettes.

VI.A.1. Constitutive Expression

35S Promoter. The CaMV 35S promoter can be used to drive constitutive gene expression. Construction of the plasmid pCGN1761 is described in the published patent application EP 0 392 225, which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker that includes NotI and XhoI sites in addition to the existing EcoRI site. This derivative is designated pCGN1761ENX. pCGN1761ENX is useful for the cloning of cDNA sequences or gene sequences (including microbial open reading frame (ORF) sequences) within its polylinker for the purpose of their expression under the control of the 35S promoter in transgenic plants. The entire 35S promoter-gene sequence-tml terminator cassette of such a construction can be excised by HindIII, SphI, SalI, and XbaI sites 5′ to the promoter and XbaI, BamHI and BglI sites 3′ to the terminator for transfer to transformation vectors such as those described below. Furthermore, the double 35S promoter fragment can be removed by 5′ excision with HindIII, SphI, SalI, XbaI, or PstI, and 3′ excision with any of the polylinker restriction sites (EcoRI, NotI or XhoI) for replacement with another promoter.

Actin Promoter. Several isoforms of actin are known to be expressed in most cell types and consequently the actin promoter is a good choice for a constitutive promoter. In particular, the promoter from the rice ActI gene has been cloned and characterized (McElroy et al., 1990). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al., 1991). These incorporate the ActI-intron 1, AdhI 5′ flanking sequence and AdhI-intron 1 (from the maize alcohol dehydrogenase gene) and sequence from the CaMV 35S promoter. Vectors showing highest expression were fusions of 35S and ActI intron or the ActI 5′ flanking sequence and the ActI intron. Optimization of sequences around the initiating ATG (of the β-glucuronidase (GUS) reporter gene) also enhanced expression. The promoter expression cassettes described by McElroy et al., 1991 can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al., 1993).

Ubiquitin Promoter. Ubiquitin is another gene product known to accumulate in many cell types and its promoter has been cloned from several species for use in transgenic plants (e.g. sunflower by Binet et al., 1991 and maize by Christensen et al., 1989). The maize ubiquitin promoter has been developed in transgenic monocot systems and its sequence and vectors constructed for monocot transformation are disclosed in the patent publication EP 0 342 926 which is herein incorporated by reference. Taylor et al., 1993 describe a vector (pAHC25) that comprises the maize ubiquitin promoter and first intron and its high activity in cell suspensions of numerous monocotyledons when introduced via microprojectile bombardment. The ubiquitin promoter is suitable for gene expression in transgenic plants, especially monocotyledons. Suitable vectors are derivatives of pAHC25 or any of the transformation vectors described in this application, modified by the introduction of the appropriate ubiquitin promoter and/or intron sequences.

VI.A.2. Inducible Expression

Chemically Inducible PR-1a Promoter. The double 35S promoter in pCGN1761ENX can be replaced with any other promoter of choice that will result in suitably high expression levels. By way of example, one of the chemically regulatable promoters described in U.S. Pat. No. 5,614,395 can replace the double 35S promoter. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemical/pathogen regulated tobacco PR-1a promoter is cleaved from plasmid pCIB1004 (for construction, see EP 0 332 104, which is hereby incorporated by reference) and transferred to plasmid pCGN1761ENX (Uknes et al., 1992).

pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is done by cleavage with XhoI and blunting with T4 DNA polymerase, followed by cleavage with HindIII and isolation of the larger vector-terminator-containing fragment into which the pCIB1004 promoter fragment is cloned. This generates a pCGN1761ENX derivative with the PR-1a promoter and the tml terminator and an intervening polylinker with unique EcoRI and NotI sites. The selected coding sequence can be inserted into this vector, and the fusion products (i.e., promoter-gene-terminator) can subsequently be transferred to any selected transformation vector, including those described below. Various chemical regulators can be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395, herein incorporated by reference.

Wound-Inducible Promoters. Wound-inducible promoters can also be suitable for gene expression. Numerous such promoters have been described (e.g. Xu et al., 1993; Logemann et al., 1989; Rohrmeier & Lehle, 1993; Firek et al., 1993; Warner et al., 1993) and all are suitable for use with the presently disclosed subject matter. Logemann et al., 1989 describe the 5′ upstream sequences of the dicotyledonous potato wunl gene. Xu et al., 1993 show that a wound-inducible promoter from the dicotyledon potato (pin2) is active in the monocotyledon rice. Further, Rohrmeier & Lehle, 1993 describe the cloning of the maize WipI cDNA, which is wound induced and which can be used to isolate the cognate promoter using standard techniques. Similarly, Firek et al., 1993 and Warner et al., 1993 have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

VI.A.3. Tissue-Specific Expression

Root Promoter. Another pattern of gene expression is root expression. A suitable root promoter is described by de Framond, 1991 and also in the published patent application EP 0 452 269, which is herein incorporated by reference. This promoter is transferred to a suitable vector such as pCGN1761ENX for the insertion of a selected gene and subsequent transfer of the entire promoter-gene-terminator cassette to a transformation vector of interest.

Pith Promoter. PCT International Publication No. WO 93/07278, which is herein incorporated by reference, describes the isolation of the maize trpA gene, which is preferentially expressed in pith cells. The gene sequence and promoter extending up to −1726 basepairs (bp) from the start of transcription are presented. Using standard molecular biological techniques, this promoter, or parts thereof, can be transferred to a vector such as pCGN1761 where it can replace the ³⁵S promoter and be used to drive the expression of a foreign gene in a pith-preferred manner. In fact, fragments containing the pith-preferred promoter or parts thereof can be transferred to any vector and modified for utility in transgenic plants.

Leaf Promoter. A maize gene encoding phosphoenol carboxylase (PEPC) has been described by Hudspeth & Grula, 1989. Using standard molecular biological techniques the promoter for this gene can be used to drive the expression of any gene in a leaf-specific manner in transgenic plants.

VI.B. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV ³⁵S terminator, the tml terminator, the nopaline synthase terminator, and the pea rbcS E9 terminator. With regard to RNA polymerase III terminators, these terminators typically comprise a run of 5 or more consecutive thymidine residues. In some embodiments, an RNA polymerase III terminator comprises the sequence TTTTTTT. These can be used in both monocotyledons and dicotyledons.

VI.C. Sequences for the Enhancement or Regulation of Expression

Numerous sequences have been found to enhance the expression of an operatively lined nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., 1987). In the same experimental system, the intron from the maize bronze1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al., 1987; Skuzeski et al., 1990).

VII. Recombinant Expression Vectors

Suitable expression vectors that can be used include, but are not limited to, the following vectors or their derivatives: yeast vectors, bacteriophage vectors (e.g., lambda phage), and plasmid and cosmid DNA vectors.

Numerous vectors available for plant transformation can be prepared and employed in the present methods. Exemplary vectors include pCIB200, pCIB2001, pCIB10, pCIB3064, pSOG19, pSOG35, and pSIT, each described herein. The selection of vector can depend upon the chosen transformation technique and the target species for transformation.

VII.A. Agrobacterium Transformation Vectors

Many vectors are available for transformation using Agrobacterium tumefaciens. These typically carry at least one T-DNA border sequence and include vectors such as pBIN19 (Bevan, 1984) and pXYZ. Below, the construction of two typical vectors suitable for Agrobacterium transformation is described.

pCIB200 and pCIB2001. The binary vectors pcIB200 and pCIB2001 are used for the construction of recombinant vectors for use with Agrobacterium and are constructed in the following manner. pTJS75kan is created by NarI digestion of pTJS75 (Schmidhauser & Helinski, 1985) allowing excision of the tetracycline-resistance gene, followed by insertion of an AccI fragment from pUC4K carrying an NPTII (Messing & Vierra, 1982; Bevan et al., 1983; McBride et al., 1990). XhoI linkers are ligated to the EcoRV fragment of PCIB7 which contains the left and right T-DNA borders, a plant selectable nos/nptII chimeric gene and the pUC polylinker (Rothstein et al., 1987), and the XhoI-digested fragment are cloned into SalI-digested pTJS75kan to create pCIB200 (see also EP 0 332 104, herein incorporated by reference).

pCIB200 contains the following unique polylinker restriction sites: EcoRI, SstI, KpnI, BglII, XbaI, and SalI. pCIB2001 is a derivative of pCIB200 created by the insertion into the polylinker of additional restriction sites. Unique restriction sites in the polylinker of pCIB2001 are EcoRI, SstI, KpnI, BglII, XbaI, SalI, MluI, BclI, AvrlI, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

pCIB10 and Hygromycin Selection Derivatives thereof. The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al., 1987. Various derivatives of pCIB10 are constructed which incorporate the gene for hygromycin B phosphotransferase described by Gritz et al., 1983. These derivatives enable selection of transgenic plant cells on hygromycin only (pCIB743), or hygromycin and kanamycin (pCIB715, pCIB717).

pSIT. pSIT is an Agrobacterium binary vector that can be used to stably express exogenous nucleic acids (for example, miRNAs and/or siRNAs) in plants. pSIT encodes two transcription units. The first is a transcription unit encoding a selectable marker under control of a promoter-transcription terminator pair that functions in plants cells. The second transcription unit encodes the gene of interest (for example, an miRNAs and/or siRNA) under the control of a second promoter-transcription terminator pair, which specifically directs the transcription to generate a functional miRNAs and/or siRNA in plant cells and which can be the same or different than the one operatively linked to the selectable marker. In some embodiments, an miRNAs and/or siRNA is operatively linked to an RNA polymerase III promoter (for example, the At7SL4 promoter) and the RNA-polymerase-III-recognized transcription terminator (for example, TTTTTTT). The integration of the miRNAs and/or siRNA cassette is guaranteed if the transformants survived through the antibiotic selection process due to the expression of the selection marker gene incorporated in the binary vector. The hpt (hygromycin phosphotransferase) selection marker gene is operatively under the control of a pair of Pnos promoter and Nos terminator. Other pairs of promoter and terminator that can drive selection marker gene expression also are suitable for the purpose.

VII.B. Other Plant Transformation Vectors

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. polyethylene glycol (PEG) and electroporation), and microinjection. The choice of vector can depend on the technique chosen for the species being transformed. Below, the construction of typical vectors suitable for non-Agrobacterium transformation is described.

pCIB3064. pCIB3064 is a pUC-derived vector suitable for direct gene transfer techniques in combination with selection by the herbicide BASTA® (or phosphinothricin). The plasmid pCIB246 comprises the CaMV 35S promoter in operational fusion to the E. coli β-glucuronidase (GUS) gene and the CaMV 35S transcriptional terminator and is described in PCT International Publication No. WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025.

The GUS gene is then excised from pCIB3025 by digestion with SalI and SacI, the termini rendered blunt and religated to generate plasmid pCIB3060. The plasmid pJIT82 is obtained from the John Innes Centre (Norwich, United Kingdom), and a 400 bp SmaI fragment containing the bar gene from Streptomyces viridochromogenes is excised and inserted into the HpaI site of pCIB3060 (Thompson et al., 1987). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

pSOG19 and pSOG35. pSOG35 is a transformation vector that utilizes the E. coli gene dihydrofolate reductase (DHFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech, Palo Alto, Calif., United States of America) that comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry a β-lactamase gene from the pUC vector for ampicillin resistance and have HindIII, SphI, PstI and EcoRI sites available for the cloning of foreign substances.

VII.C. Selectable Markers

For certain target species, different antibiotic or herbicide selection markers can be preferred. Selection markers used routinely in transformation include the nptII gene, which confers resistance to kanamycin and related antibiotics (Messing & Vierra, 1982; Bevan et al., 1983), the bar gene, which confers resistance to the herbicide phosphinothricin (White et al., 1990; Spencer et al., 1990), the hph gene, which confers resistance to the antibiotic hygromycin (Blochlinger & Diggelmann, 1984), the dhfr gene, which confers resistance to methotrexate (Bourouis & Jarry, 1983), and the 5-enolpyruvylshikimate-3-phosphate (EPSP) synthase gene, which confers resistance to glyphosate (U.S. Pat. Nos. 4,940,935 and 5,188,642).

VIII. Transformation

Once a nucleic acid sequence of the presently disclosed subject matter has been cloned into an expression system, it is transformed into a plant cell. The receptor and target expression cassettes of the presently disclosed subject matter can be introduced into the plant cell in a number of art-recognized ways. Methods for regeneration of plants are also well known in the art. For example, Ti plasmid vectors have been utilized for the delivery of foreign DNA, as have direct DNA uptake, liposomes, electroporation, microinjection, and microprojectiles. In addition, bacteria from the genus Agrobacterium can be utilized to transform plant cells.

The presently disclosed subject matter also provides a method for stably modulating expression of a gene in a plant. In some embodiments, the method comprises (a) transforming a plurality of plant cells with a vector comprising a nucleic acid sequence encoding a microRNA (miRNA) operatively linked to a promoter and a transcription termination sequence; (b) growing the plant cells under conditions sufficient to select for a plurality of transformed plant cells that have integrated the vector into their genomes; (c) screening the plurality of transformed plant cells for expression of the miRNA encoded by the vector; (d) selecting a transformed plant cell that expresses the miRNA; and (e) regenerating the plant from the transformed plant cell that expresses the miRNA, whereby expression of the plant gene is stably modulated. In some embodiments, the method comprises (a) transforming a plurality of plant cells with an Agrobacterium tumefaciens binary vector comprising (i) a nucleic acid sequence encoding a selectable marker; and (ii) a nucleic acid sequence encoding a microRNA (miRNA) operatively linked to a promoter and a transcription termination sequence; (b) treating the plant cells with a drug under conditions sufficient to kill those plant cells that did not receive the binary vector, wherein the selectable marker provides resistance to the drug, to create a first plurality of transformed plant cells; (c) growing the first plurality of transformed plant cells under conditions sufficient to select for a second plurality of transformed plant cells that have integrated the binary vector into their genomes; (d) screening the second plurality of transformed plant cells for expression of the miRNA encoded by the expression vector; (e) selecting a transformed plant cell that expresses the miRNA; and (f) regenerating the plant from the transformed plant cell that expresses the miRNA, whereby expression of the gene in the plant is stably modulated.

The presently disclosed subject matter is based on the introduction of a stable and heritable miRNAs and/or siRNAs into plant cells to specifically manipulate a gene of the interest. As disclosed herein, this concept has been demonstrated through Agrobacterium transformation, but would also be applicable to other approaches for transformation, such as bombardment. Thus, it should be understood that the mechanism of transformation of a plant cell is not limited to the Agrobacterium-mediated techniques disclosed in certain embodiments herein. Any transformation technique that results in stable expression of a nucleic acid (for example, an miRNAs and/or siRNA) of the presently disclosed subject matter can be employed with the methods disclosed herein. Below are descriptions of representative techniques for transforming both dicotyledonous and monocotyledonous plants, as well as a representative plastid transformation technique.

VIII.A. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation-mediated uptake, particle bombardment-mediated delivery, or microinjection. Examples of these techniques are disclosed in Paszkowski et al., 1984; Potrykus et al., 1985; Reich et al., 1986; and Klein et al., 1987. In each case the transformed cells are regenerated to whole plants using standard techniques known in the art.

Agrobacterium-mediated transformation is a useful technique for transformation of dicotyledons because of its high efficiency of transformation and its broad utility with many different species. Agrobacterium transformation typically involves the transfer of the binary vector carrying the foreign DNA of interest (e.g. pSIT) to an appropriate Agrobacterium strain that can depend on the complement of vir genes carried by the host Agrobacterium strain either on a co-resident Ti plasmid or chromosomally (e.g. strain C58 or strains pCIB542 for pCIB200 and pCIB2001; Uknes et al., 1993). The transfer of the recombinant binary vector to Agrobacterium is accomplished by a triparental mating procedure using E. coli carrying the recombinant binary vector, a helper E. coli strain that carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, 1988).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050; 5,036,006; and 5,100,792; all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue.

VIII.B. Transformation of Monocotyledons

Transformation of most monocotyledon species has now also become routine. Exemplary techniques include direct gene transfer into protoplasts using PEG or electroporation, and particle bombardment into callus tissue. Transformations can be undertaken with a single DNA species or multiple DNA species (i.e., co-transformation), and both these techniques are suitable for use with the presently disclosed subject matter. Co-transformation can have the advantage of avoiding complete vector construction and of generating transgenic plants with unlinked loci for the gene of interest and a selectable marker, enabling the removal of the selectable marker in subsequent generations, should this be regarded as desirable. However, a disadvantage of the use of co-transformation is the less than 100% frequency with which separate DNA species are integrated into the genome (Schocher et al., 1986).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al., 1990 and Fromm et al., 1990 have published techniques for transformation of A188-derived maize line using particle bombardment. Furthermore, WO 93/07278 and Koziel et al., 1993 describe techniques for the transformation of elite inbred lines of maize by particle bombardment. This technique utilizes immature maize embryos of 1.5-2.5 mm length excised from a maize ear 14-15 days after pollination and a PDS-1000He biolistic particle delivery device (DuPont Biotechnology, Wilmington, Del., United States of America) for bombardment.

Transformation of rice can also be undertaken by direct gene transfer techniques utilizing protoplasts or particle bombardment. Protoplast-mediated transformation has been disclosed for Japonica-types and Indica-types (Zhang et al., 1988; Shimamoto et al., 1989; Datta et al., 1990). Both types are also routinely transformable using particle bombardment (Christou et al., 1991). Furthermore, WO 93/21335 describes techniques for the transformation of rice via electroporation.

Patent Application EP 0 332 581 describes techniques for the generation, transformation, and regeneration of Pooideae protoplasts. These techniques allow the transformation of Dactylis and wheat. Furthermore, wheat transformation has been disclosed in Vasil et al., 1992 using particle bombardment into cells of type C long-term regenerable callus, and also by Vasil et al., 1993 and Weeks et al., 1993 using particle bombardment of immature embryos and immature embryo-derived callus.

A representative technique for wheat transformation, however, involves the transformation of wheat by particle bombardment of immature embryos and includes either a high sucrose or a high maltose step prior to gene delivery. Prior to bombardment, embryos (0.75-1 mm in length) are plated onto MS medium with 3% sucrose (Murashige & Skoog, 1962) and 3 mg/l 2,4-dichlorophenoxyacetic acid (2,4-D) for induction of somatic embryos, which is allowed to proceed in the dark. On the chosen day of bombardment, embryos are removed from the induction medium and placed onto the osmoticum (i.e., induction medium with sucrose or maltose added at the desired concentration, typically 15%). The embryos are allowed to plasmolyze for 2-3 hours and are then bombarded. Twenty embryos per target plate are typical, although not critical. An appropriate gene-carrying plasmid (such as pCIB3064 or pSG35) is precipitated onto micrometer size gold particles using standard procedures. Each plate of embryos is shot with the DuPont biolistics helium device using a burst pressure of about 1000 pounds per square inch (psi) using a standard 80 mesh screen. After bombardment, the embryos are placed back into the dark to recover for about 24 hours (still on osmoticum). After 24 hours, the embryos are removed from the osmoticum and placed back onto induction medium where they stay for about a month before regeneration. Approximately one month later the embryo explants with developing embryogenic callus are transferred to regeneration medium (MS+1 mg/liter naphthaleneacetic acid (NAA), 5 mg/liter GA), further containing the appropriate selection agent (10 mg/l BASTA® in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been disclosed. See WO 94/00977 and U.S. Pat. No. 5,591,616, both of which are incorporated herein by reference. See also Negrotto et al., 2000, incorporated herein by reference. Like other Agrobacterium-mediated binary vector system used for the transformation of monocotyledons, pSIT can also be employed to modify monocotyledons.

VIII.C. Transformation of Plastids

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif., United States of America) coated with DNA from representative plasmids essentially as disclosed (Svab & Maliga, 1993). Bombarded seedlings are incubated on T medium for two days after which leaves are excised and placed abaxial side up in bright light (350-500 μmol photons/m²/s) on plates of RMOP medium (Svab et al., 1990) containing 500 μg/ml spectinomycin dihydrochloride (Sigma, St. Louis, Mo., United States of America). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook & Russell, 2001). BamHI/EcoRI-digested total cellular DNA is separated on 1% Tris-borate-EDTA (TBE) agarose gels, transferred to nylon membranes (Amersham Biosciences, Piscataway, N.J., United States of America) and probed with ³²P-labeled random primed DNA sequences corresponding to a 0.7 kb BamHI/HindIII DNA fragment from pC8 containing a portion of the rps7/12 plastid targeting sequence. Homoplasmic shoots are rooted aseptically on spectinomycin-containing MS/IBA medium (McBride et al., 1994) and transferred to the greenhouse.

IX. Plants. Breeding, and Seed Production

IX.A. Plants

The presently disclosed subject matter also provides plants comprising the disclosed compositions. In some embodiments, the plant is characterized by a modification of a phenotype or measurable characteristic of the plant, the modification being. attributable to the presence of an expression cassette comprising a nucleic acid molecule of the presently disclosed subject matter. In some embodiments, the modification involves, for example, nutritional enhancement, increased nutrient uptake efficiency, enhanced production of endogenous compounds, or production of heterologous compounds. In some embodiments, the modification includes having increased or decreased resistance to an herbicide, environmental stress, or a pathogen. In some embodiments, the modification includes having enhanced or diminished requirement for light, water, nitrogen, or trace elements. In some embodiments, the modification includes being enriched for an essential amino acid as a proportion of a polypeptide fraction of the plant. In some embodiments, the polypeptide fraction can be, for example, total seed polypeptide, soluble polypeptide, insoluble polypeptide, water-extractable polypeptide, and lipid-associated polypeptide. In some embodiments, the modification includes overexpression, underexpression, antisense modulation, sense suppression, inducible expression, inducible repression, or inducible modulation of a gene. In alternative embodiments, the modifications can include decreased or increased lignin content, lignin composition and/or structure changes, decreased or increased cellulose content, crystallinity and degree of polymerization (DP) changes, fiber property and morphology modifications, and/or increased resistance to pathogens, common diseases, and environment stresses in a tree.

IX.B. Breeding

The plants obtained via transformation with a nucleic acid sequence of the presently disclosed subject matter can be any of a wide variety of plant species, including monocots and dicots, and angiosperms and gymnosperms; however, the plants used in the method for the presently disclosed subject matter are selected in some embodiments from the list of agronomically important target crops set forth hereinabove. The modification of expression of a gene in accordance with the presently disclosed subject matter in combination with other characteristics important for production and quality can be incorporated into plant lines through breeding. Breeding approaches and techniques are known in the art. See e.g., Welsh, 1981; Wood, 1983; Mayo, 1987; Singh, 1986; Wricke & Weber, 1986.

The genetic properties engineered into the transgenic seeds and plants disclosed above are passed on by sexual reproduction or vegetative growth and can thus be maintained and propagated in progeny plants. Generally, maintenance and propagation make use of known agricultural methods developed to fit specific purposes such as tilling, sowing, or harvesting. Specialized processes such as hydroponics or greenhouse technologies can also be applied. As the growing crop is vulnerable to attack and damage caused by insects or infections as well as to competition by weed plants, measures are undertaken to control weeds, plant diseases, insects, nematodes, and other adverse conditions to improve yield. These include mechanical measures such as tillage of the soil or removal of weeds and infected plants, as well as the application of agrochemicals such as herbicides, fungicides, gametocides, nematicides, growth regulants, ripening agents, and insecticides.

Use of the advantageous genetic properties of the transgenic plants and seeds according to the presently disclosed subject matter can further be made in plant breeding, which aims at the development of plants with improved properties such as tolerance of pests, herbicides, or abiotic stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants.

Depending on the desired properties, different breeding measures are taken. The relevant techniques are well known in the art and include, but are not limited to, hybridization, inbreeding, backcross breeding, multi-line breeding, variety blend, interspecific hybridization, aneuploid techniques, etc. Hybridization techniques can also include the sterilization of plants to yield male or female sterile plants by mechanical, chemical, or biochemical means. Cross-pollination of a male sterile plant with pollen of a different line assures that the genome of the male sterile but female fertile plant will uniformly obtain properties of both parental lines. Thus, the transgenic seeds and plants according to the presently disclosed subject matter can be used for the breeding of improved plant lines that, for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions (for example, drought).

IX.C. Seed Production

Embodiments of the presently disclosed subject matter also provide seed from plants modified using the disclosed methods.

In seed production, germination quality, and uniformity of seeds are essential product characteristics. As it is difficult to keep a crop free from other crop and weed seeds, to control seedborne diseases, and to produce seed with good germination, fairly extensive and well-defined seed production practices have been developed by seed producers who are experienced in the art of growing, conditioning, and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (tetramethylthiuram disulfide; TMTD®; available from R. T. Vanderbilt Company, Inc., Norwalk, Conn., United States of America), methalaxyl (APRON XL®; available from Syngenta Corp., Wilmington, Del., United States of America), and pirimiphos-methyl (ACTELLIC®; available from Agriliance, LLC, St. Paul, Minn., United States of America). If desired, these compounds are formulated together with further carriers, surfactants, and/or application-promoting adjuvants customarily employed in the art of formulation to provide protection against damage caused by bacterial, fungal, or animal pests. The protectant coatings can be applied by impregnating propagation material with a liquid formulation or by coating with a combined wet or dry formulation. Other methods of application are also possible such as treatment directed at the buds or the fruit.

X. Transgenic Plants

A “transgenic plant” is one that has been genetically modified to contain and express an miRNA and/or an siRNA. A transgenic plant can be genetically modified to contain and express at least one homologous or heterologous DNA sequence operatively linked to and under the regulatory control of transcriptional control sequences which function in plant cells or tissue or in whole plants. As used herein, a transgenic plant also refers to progeny of the initial transgenic plant where those progeny contain and are capable of expressing the homologous or heterologous coding sequence under the regulatory control of the plant-expressible transcription control sequences described herein. Seeds containing transgenic embryos are encompassed within this definition as are cuttings and other plant materials for vegetative propagation of a transgenic plant.

When plant expression of a homologous or heterologous gene or coding sequence of interest is desired, that coding sequence is operatively linked in the sense orientation to a suitable promoter and advantageously under the regulatory control of DNA sequences which quantitatively regulate transcription of a downstream sequence in plant cells or tissue or in planta, in the same orientation as the promoter, so that a sense (i.e., functional for translational expression) mRNA is produced. A transcription termination signal, for example, as polyadenylation signal, functional in a plant cell is advantageously placed downstream of an miRNA- and/or siRNA-encoding sequence, and a selectable marker which can be expressed in a plant, can be covalently linked to the inducible expression unit so that after this DNA molecule is introduced into a plant cell or tissue, its presence can be selected and plant cells or tissue not so transformed will be killed or prevented from growing.

Where tissue specific expression of the plant-expressible miRNA and/or siRNA coding sequence is desired, the skilled artisan can choose from a number of well-known sequences to mediate that form of gene expression as disclosed herein. Environmentally regulated promoters are also well known in the art and are disclosed herein, and the skilled artisan can choose from well-known transcription regulatory sequences to achieve the desired result.

Summarily, the presently disclosed subject matter can be employed, among other applications, to perform the following:
1. Specifically downregulate a target gene in a stable and heritable manner;
2. Enhance target gene expression by downregulating negative regulators;
3. Regulate transcriptional activity of a target promoter; and
4. Molecular regulation through miRNA-induced silencing signal movement.

### EXAMPLES

The following Examples have been included to illustrate modes of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Isolation of Small RNAs from P. trichocarpa

Total RNA was isolated from developing xylem tissue of P. trichocarpa or P. taeda, from pooled tension- and compression-stressed developing xylem of P. trichocarpa stems (bend for 4 days), from P. trichocarpa in vitro plants, or from pooled P. trichocarpa in vitro plants wit or without exposure to cold (4° C. for 24 hours), heat (37° C. for 24 hours), dehydration (draught for 14 hours), salinity (300 mM NaCl for 14 hours), or water (plants covered with water for 14 hours), using the cetyl trimethyl ammonium bromide (CTAB) method as described in Chang et al. 1993. Cloning of miRNAs was performed as described (Lau et al., 2001; Lagos-Quintana et al., 2002; Elbashir et al., 2001b). Briefly, isolated total RNA was separated on a 12% denaturing polyacrylamide gel. A band corresponding to RNA of about 16-36 nt in size was excised and the RNA was recovered from the gel slice. The recovered RNA was dephosphorylated with alkaline phosphatase, and a 5′-phosphorylated-3′-adaptor oligonucleotide with the sequence 5′-CTGTAGGCACCATTCATCAC-3′ (SEQ ID NO: 155) with a 5′-phosphate and a 3′-amino-modifier C-7 (i.e. a seven-carbon spacer with a primary amino group) was then ligated to the dephosphorylated RNA. The ligated products were separated from non-ligated RNA and the adaptor oligonucleotide on a 12% denaturing polyacrylamide gel. A band corresponding to the ligation product was excised from the gel, and the ligated RNA was recovered. The RNA was phosphorylated at the 5′ end and a new 5′ adaptor oligonucleotide (5′-ATGTCGTGaggcacctgaaa-3′ (SEQ ID NO: 156; the sequence in uppercase is a DNA strand and in lowercase is an RNA strand) containing hydroxyl groups at both 5′ and 3′ ends was ligated to the 5′-phosphorylated ligation product from the previous step. The new ligation product was gel purified and eluted from the gel slice.

Reverse transcription was performed by using a RT primer (5′-GATGAATGGTGCCTAC-3′; SEQ ID NO: 157), followed by PCR using a 5′ primer (5′-GTCGTGAGGCACCTGAAA-3′; SEQ ID NO: 158) and a 3′ primer (5′-GATGAATGGTGCCTACAG-3′; SEQ ID NO: 159). The PCR product was then digested with Ban I and concatamerized using T4 DNA ligase. The products of the ligation reaction were separated on an agarose gel, and a gel slice corresponding to concatamers of a size range of larger than 500 basepairs (bp) was isolated and the nucleic acids recovered from the gel slice. The single-stranded regions of the ends of the concatamers were filled in by incubation with Taq polymerase, and the DNA product was directly ligated into the pCR2.1-TOPO® vector using the TOPO TA CLONING® kit (Invitrogen Corp., Carlsbad, Calif., United States of America).

### Example 2

### Isolation of P. trichocarpa miRNAs

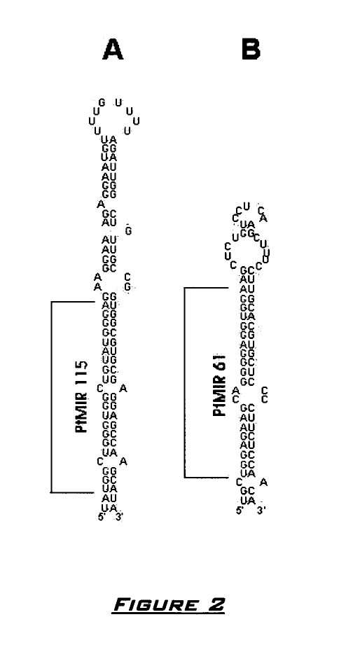

After the subcloning described in Example 1, inserts were sequenced from P. trichocarpa. After excluding sequences corresponding to rRNA, tRNA, snRNA, retrotransposons/transposons, and small RNAs with 2 nt or more mismatches with the P. trichocarpa genome, the remaining small RNA sequences and their surrounding sequences from the P. trichocarpa genome were used to predict the secondary structures of these small RNAs using the mfold program (Zuker, 2003). 52 miRNA families were identified (Table 1) based on their authentic pre-miRNA stem-loop structures (see FIG. 2, showing two examples) or their significant homology to miRNAs identified in other species.

These miRNAs were subjected to BLAST analyses against the GENBANK® database (available from the National Center for Biotechnology Information (NCBI) website) and the miRBase sequence database (available from the website of the Wellcome Trust Sanger Institute). According to the results from BLAST analyses, the cloried sequences were divided into two groups: group I and group II. Of these, 19 had either identical or highly homologous sequences to those of some Arabidopsis miRNAs (Palatnik et al., 2003; Sunkar & Zhu, 2004; see Table 1). The other 33 miRNA sequences were did not show significant homology to Arabidopsis miRNAs. Interestingly, only 3 (PtmiR 73, PtmiR 132 and PtmiR 181) of these 33 miRNAs were found in Arabidopsis, indicating that a majority of the identified P. trichocarpa xylem miRNAs are unique to wood formation.

### Example 3

### Isolation of P. taeda miRNAs

After the subcloning described in Example 1, inserts were sequenced from P. taeda. After excluding sequences corresponding to rRNA, tRNA, snRNA, and retrotransposons/transposons, the remaining small RNA sequences and their surrounding sequences from the P. taeda expressed sequence tags (ESTs) deposited in dbEST of the GENBANK® database were used to predict the secondary structures of these small RNAs using the mfold program (Zuker, 2003). 15 miRNA families were identified (Table 4, LpMIR1, LpMIR2, LpMIR7, LpMIR9, LpMIR178, LpMIR26, LpMIR27, LpMIR28, LpMIR77, LpMIR82, LpMIR89, LpMIR95, LpMIR100, LpMIR119, and LpMIR176) based on their authentic pre-miRNA stem-loop structures or their significant homology to miRNAs identified in other species.

These miRNAs were subjected to BLAST analyses against the GENBANK® database and the miRBase sequence database (available from the website of the Wellcome Trust Sanger Institute. According to the results from BLAST analyses, the cloned sequences were divided into two groups: group I and group II. Of these, 3 had either identical or highly homologous sequences to those of some Arabidopsis miRNAs (Palatnik et al., 2003; Sunkar & Zhu, 2004; see Table 1). The other 12 miRNA sequences did not show significant homology to Arabidopsis miRNAs.

### Example 4

### Identification of Additional miRNAs from P. trichocarpa

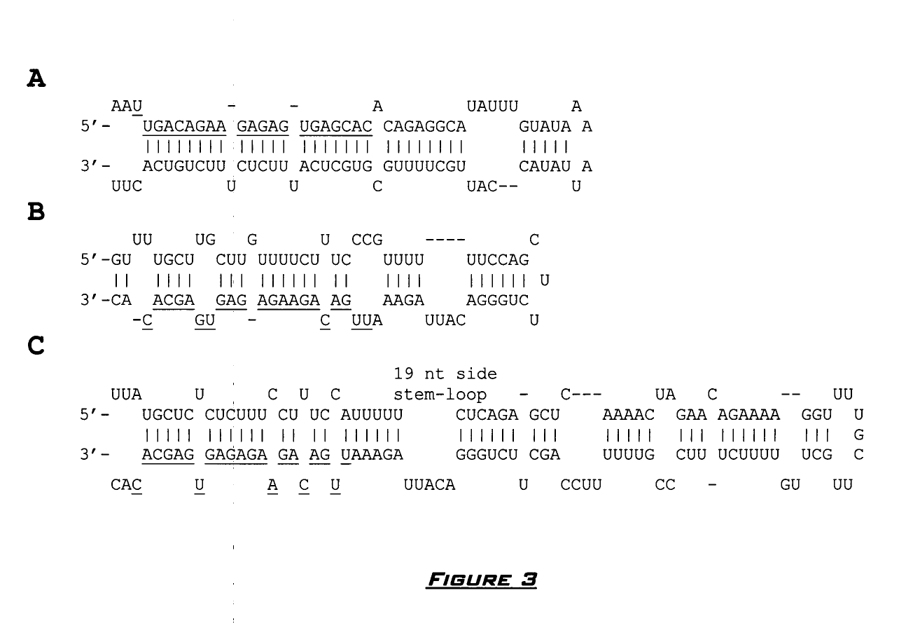

When the genomic sequences surrounding the closely related homologs (i.e., the P. trichocarpa miRNAs that showed 1 and 2 mismatches to the isolated P. trichocarpa miRNAs) were analyzed, 66 additional loci were identified. Some of the isolated miRNA showed high homology to each other, for example, PtmiR 71 and PtmiR 142 (Table 1), resulting in 3 loci each of which had a sequence showing high homology to two miRNAs. Among these 3 loci, one locus had a sequence showing a 1 nt mismatch to both PtmiR 71 and PtmiR 142, and the other two loci each had a sequence showing a 1 nt mismatch to PtmiR 71 and 2 nt mismatch to PtmiR 142. Moreover, one locus (PtMIR 156-1) harboring an miRNA with two mismatches to PtmiR 156 was able to form stable stem-loop structures with the miRNA sequences present in either the 5′ or the 3′ arm, and two stem-loop structures (one is shorter and another is longer) were found when the miRNA was present in the 3′ arm (see FIG. 3). Moreover, the four PtmiR 71 genes had a sequence showing a 1 nt mismatch to PtmiR 142.

### Example 5

### Identification of Additional miRNAs from P. taeda

When the EST sequences surrounding the closely related homologs (i.e., the P. taeda miRNAs that showed 1 and 2 mismatches to the isolated P. taeda miRNAs) were analyzed, 17 additional loci were identified (Table 4). Whether any of the P. trichocarpa miRNA families are present in P. taeda has also been investigated. By allowing zero to two nucleotide substitutions, the sequences of some PtmiRNAs were searched against the P. taeda EST database to identify their P. taeda homologs and the surrounding sequences. Analysis of the LpmiRNA sequence-containing loci in P. taeda by the mfold program (Zuker, 2003) resulted in the identification of 5 novel P. taeda miRNA families (LpMIR170, LpMIR274, LpMIR277, LpMIR279, and LpMIR472. representing by 10 additional loci (Table 4).

### Example 6

### Identification of Potential miRNA Target Genes

Based on the miRNA sequences, target genes for the isolated Populus trichocarpa miRNAs were identified by searching the genome and predicted transcripts of P. trichocarpa with the program PATSCAN (Dsouza & Larsen, 1997), which can be used to identify mRNAs capable of base pairing with one of the miRNAs with a score of 3.0 or less (see Jones-Rhoades et al., 2004 for detail description for scoring method). The same method was used to identify potenitial target genes for miRNAs isolated from Pinus taeda by seaching throught the Pine Gene Index Release 6.0 produced by The Institute for Genomic Research (TIGR; available at the website of TIGR). This included potential target genes for 35 poplar and pine miRNAs that did not show any homology to Arabidopsis miRNAs (Table 2).

### Discussion of Example 6

The predicted targets comprise, in general, regulatory and defense related genes. While some of the targets are associated with development, and/or with cellulose biosynthesis, many of them are implicated in the lignin biosynthesis network. For example, LpMIR 178 was found to target a cellulose synthase, an enzyme involved in the synthesis of the backbone of the cell wall. The predicted target of PtmiR 6 encodes a UVR8 protein, which positively regulates phenylpropanoid metabolism associated with cinnamate 4-hydroxylase (C4H) in response to UV-B induction (Hu et al., 1998; Jin et al., 2000; Kliebenstein et al., 2002). Also, PtmiR 241 and PtmiR 13 each targets genes that encodes laccases and a mononuclear blue copper protein family member. These two protein families were suggested to be involved in lignin formation (Nersissian et al., 1999). A common target of PtmiR 29, 71, and 142 encode MYB factor proteins, which are transcription factors known to bind promoters of a variety of lignin biosynthetic pathway genes encoding, for example, PAL, C4H, 4-coumaroyl-CoA ligase (4CL), 5-hydroxyconiferaldehyde O-methyltransferase (COMT) and cinnamyl alcohol dehydrogenase (CAD; Tamagnone et al., 1998; Borevitz et al., 2000). Down- or up-regulating these genes results in drastic lignin reduction or augmentation, respectively (Tamagnone et al., 1998; Borevitz et al., 2000). Suppression of a LIM protein, a predicted target of PtmiR 172, also inhibited PAL, 4CL, and CAD expression, resulting in significant lignin reduction (Kawaoka et al., 2000; Kawaoka & Ebinuma, 2001). The most striking discovery was the perfect sequence complementarity between PtmiR 172 and another target, the G lignin-specific CAD, suggesting a role for PtmiR 172 in a negative feedback mechanism in, perhaps, controlling the preferential biosynthesis of specific lignin types.

### Example 7

### Expression of PtmiR Nucleic Acids in P. trichocarpa Tissues

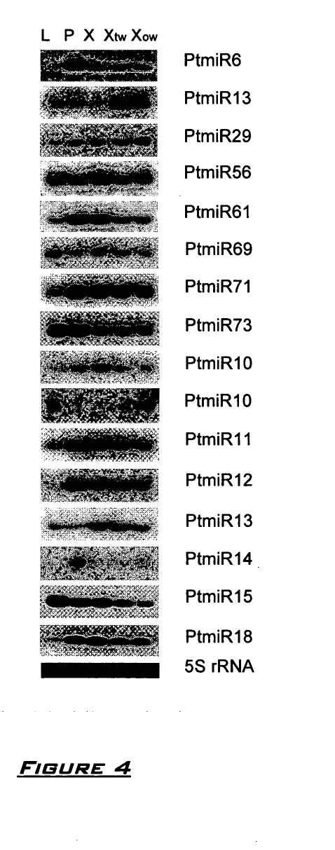

The expression of some of the PtmiRs in various P. trichocarpa tissues was characterized by Northern analysis (FIG. 4). This included xylem tissues suffering from tension stress from tension wood (TW) and from compression stress from stem wood opposite to TW, called opposite wood (OW). TW and OW can be easily created by bending the tree stem. The tested PtmiR s are all expressed at some level in woody tissues (for example, phloem, secondary growth, tension wood, and opposite wood).

Northern hybridization was performed essentially as described in Hutvágner et al., 2000. Total RNA (30 μg) was denatured for 10 minutes at 65-70° C., separated on a 12% polyacrylamide/8 M urea gel (Amersham Biosciences, Piscataway, N.J., United States of America) in a PROTEAN II apparatus (Bio-Rad Laboratories, Inc., Hercules, Calif., United States of America), and electro-blotted onto a HYBOND™-N⁺ membrane (Amersham) using a Trans-Blot SD Semi-Dry Electrophoretic Transfer Cell (Bio-Rad). After UV cross-linking and air drying, blots were prehybridized in ULTRAHYB™-Oligo hybridization buffer (Ambion Inc., Austin, Tex., United States of America), and hybridized with [γ-³²P]ATP-labeled DNA oligonucleotides complementary to small RNA sequences. The hybridization was carried out overnight in ULTRAHYB™-Oligo buffer at 37° C. After hybridization, blots were washed twice with a wash buffer containing 2×SSC and 0.5% SDS at 37° C. for 0.5 hour each time. Signals were visualized by autoradiography at −80° C.

Interestingly, while PtmiR 29 is expressed strongly in xylem, its Arabidopsis homolog (AtmiR159) was not expressed in Arabidopsis stem, as reported by Park et al. See Park et al., 2002. Instead, AtmiR159 was found most highly expressed in Arabidopsis leaves, contrasting directly with the considerably lower expression of its P. trichocarpa homolog, PtmiR 29, in leaves than in lignifying tissues. Thus, miRNA sequence conservation between plant species might not suggest conserved miRNA functions in these species.

### Discussion of Example 7

Based on the expression patterns of these PtmiRs showing high levels of transcripts in wood forming tissues, xylem in particular, and on the predicted target miRNAs (see Table 2), the disclosed PtmiRs might play significant roles in regulating wood development in plants. The expression patterns and predicted target miRNA functions also point to critical roles for these PtmiRs in regulating lignin, cellulose, and hemicellulose biosynthesis. The strong expression of PtmiR 73 in leaf together with its target gene function associated with disease resistance (see Table 2) is direct evidence for the involvement of PtmiR 73 in the regulation of disease and stress tolerance.

### Example 8

### Identification of Potential siRNA Target Sites in any RNA Sequence

The sequence of an RNA target of interest, such as a plant mRNA transcript, is screened for target sites, for example by using a computer-based folding algorithm. In a non-limiting example, the sequence of a gene or RNA gene transcript derived from a database, such as the GENBANK® database or any other database containing nucleotide sequence data (for example, a database containing sequence data from plants, such as Arabidopsis, P. trichocarpa, rice, etc.) is used to generate siRNA targets having complementarity to the target. Such sequences can be obtained from a database, or can be determined experimentally as disclosed herein and/or known in the art. Target sites that are known include, for example, those target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well.

Target sites can include single-stranded regions of miRNA precursors. As disclosed herein and shown in FIG. 2, miRNA precursors adopt a stem-loop structure consisting of double-stranded and single-stranded regions. siRNA molecules are designed that hybridize to the double-stranded or single stranded regions of an miRNA precursor or to the miRNA sequence, thus causing aberrant processing of the precursor and inhibiting miRNA production.

Various parameters can be used to determine which sites are the most suitable target sites within the target RNA sequence. These parameters include, but are not limited to secondary or tertiary RNA structure, the nucleotide base composition of the target sequence, the degree of homology between various regions of the target sequence, and the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. In a non-limiting example, anywhere from 1 to 1000 target sites are chosen within the transcript based on the size of the siRNA construct to be used. High throughput screening assays can be developed for screening siRNA molecules using methods known in the art, such as with multi-well or multi-plate assays to determine efficient reduction in target gene expression.

### Example 9

### siRNA-Mediated Modulation of Gus Gene Expression in Transgenic Tobacco Design of siRNAs Directed Against the GUS Gene

Based on the standard design rules (Elbashir et al., 2002) two 19 nt sequences (designated GT1 and GT2) targeting two distinct sites in the GUS mRNA were selected for constructing the expression vectors. Individual siRNA templates comprised the 19 nt fragment linked via a 9 nt spacer to the reverse complement of the same 19 nt sequence. Each template was cloned into a vector comprising a human H1 RNA transcription unit under the control of its cognate gene promoter (FIG. 9). The resulting transcript was predicted to adopt an inverted hairpin RNA structure containing one (for GT1) or two (for GT2) 3′ overhanging uridines, giving rise to siRNA-like transcripts containing GT1 or GT2 sequences (FIG. 9). As shown in FIG. 9, GT1 produces an siRNA-like transcript comprising SEQ ID NO: 172—9 nt spacer—SEQ ID NO: 173 (bottom left), and GT2 produces a transcript comprising SEQ ID NO 174—9 nt spacer—SEQ ID NO: 175.

RNA Silencing with Human H1 Promoter-Containing Constructs. Agrobaterium tumefaciens C58 cells were transformed with the GT1 and GT2 vectors and used to transform a transgenic tobacco line expressing a GUS transgene (Hu et al., 1998). To transfer to tobacco, GUS-containing tobacco leaf disks were infected with the Agrobacterium C58 strain harboring the siRNA construct. Transformants were selected on MS104 containing 25 mg/L hygromycin and 300 mg/L claforan. The hygromycin-resistant shoots were placed on hormone-free MSO agar medium containing 25 mg/L hygromycin and 300 mg/L claforan for root regeneration, and transgenic tobacco seedlings were planted in soil and grown in a greenhouse.

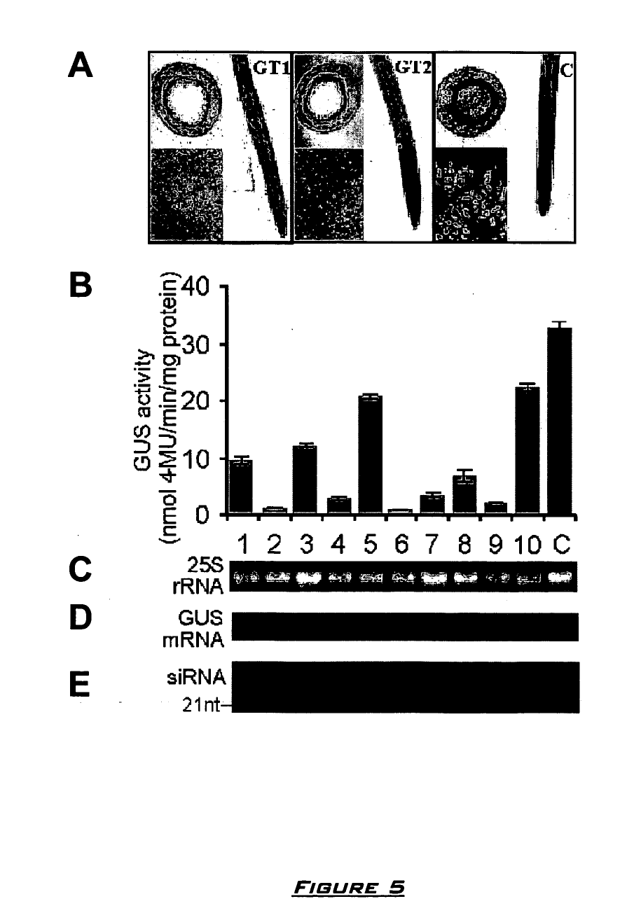

Twenty-three transgenic plants were produced from the GT1 construct and nineteen from the GT2 construct. Transgenic plants and GUS-carrying control plants were characterized at about one month old. The stem, leaf, and root of a majority of the GT1 and GT2 transgenics exhibited either reduced or no GUS staining (FIG. 5A). Assays of GUS protein activity in leaves indicated that 74% of the GT1 transgenics had a reduction in GUS activity ranging from 12 to 94%, and 84% of the GT2 transgenics exhibited a reduction in GUS activity of 31 to 97%. The reduction in GUS activity (see FIG. 5B) reflected diminished GUS mRNA levels in these plants (see FIGS. 5C and 5D). Small discrete RNAs of about 21 nt in length were present in the transgenic lines having reduced GUS mRNA and protein activity, but absent from the control line (see FIG. 5E). Overall, the abundance of this 21 nt RNA was inversely correlated with the abundance of GUS mRNA in these plants (see FIGS. 5C and 5E).

The gene silencing efficiency appeared to be independent of the GUS mRNA target sites and of the number of uridine residues (1 vs. 2) in the engineered siRNA transcripts. Furthermore, the silencing effect remained in about 90% of the T₁ plants analyzed.

Cloning of the Arabidopsis 7SL4 Promoter. Two oligonucleotides corresponding to the promoter region of the Arabidopsis thaliana At7SL4 gene were designed based upon data present in the publicly available Arabidopsis database (see the website for the Institute for Genomic Research). These primers are SLpF (5′-GGAATTCTGCGTTTGAAGAAGA GTGTTTGA-3′; SEQ ID NO: 160) as the forward primer (with the addition of an Eco RI site at the 5′ end) and SLpR (5′-GCCCGGG AAGATCGGTTCGTGTAATATAT-3′; SEQ ID NO: 161) as the reverse primer (with addition of a Sma I site at the 5′ end). These two primers flank the At7SL4 gene promoter at both ends and were used for PCR amplification of the promoter fragment from Arabidopsis thaliana (Columbia ecotype) genomic DNA.

The PCR product amplified from Arabidopsis genomic DNA using primers SLpF and SLpR was cloned into the PCR®2.1-TOPO® system (Invitrogen Corp., Carlsbad, Calif., United States of America) and the sequence of the promoter fragment confirmed by sequencing. The resulting At7SL4 promoter clone was named pCRSLp7, and contained the following At7SL4 promoter sequence: GGAATTCTGCGTTTGAAGAAGAGTGTTTGA TGTTCTCAAGTAAGTGAGTCTTATTGGGAATAATATTAACTCATGTTCTT CTTGCATTTGATTTCTTTGCCGCTCTCTTCTTCTATCTCAAATCTGTCTCT TCAATTTCACAGTTGGGCTTTTTATTAGTCTATAATGGGACTCAAAATAA GGCTTTGGCCCACATCAAAAAGATAAGTCAAATGAAAACTAAATTCAGT CTTTTGTCCCACATCGATCACTCTACTCGTTTTGTGTTTGTTTATATATTA CACGAACCGATCTTCCCGGGC (SEQ ID NO: 162). The sequences of the SLpF and SLPR primers are underlined.

Cloning of the Arabidopsis At7SL4 Gene 3′ Non-translated Sequence. To clone the 3′-NTS of the At7SL4 gene, two oligos were synthesized based on sequence information available in the the Arabidopsis database as described hereinabove. The primers used were as follows: SLtF 5′-GTCTAGATTTTGATTTTGTTTTCCAAAACTTTCTACG-3′ (SEQ ID NO: 163), was used as the forward primer (adds an XbaI site added to the 5′ end of the 3′-NTS); and SLtR 5′-GAAGCTTGGTGTTGATCACAACGATACA-3′ (SEQ ID NO: 164) was used as the reverse primer (adds a HindIII site to the 3′ end of the 3′-NTS). PCR was employed to amplify a nucleic acid molecule comprising the 3′-NTS using these two primers and Arabidopsis thaliana (Columbia ecotype) genomic DNA. The amplified nucleic acid molecule was cloned into the PCR®2.1-TOPO® system (Invitrogen Corp.) and sequenced (plasmid referred to herein as pCRSLt2). The correct At7SL4-3′-NTS nucleotide sequence was determined to be: GTCTAGATTTTGATTTT GTTTTCCAAAACTTTCTACGCTTTTTGTTTTTGGGTTTAATGCTTTAAGAG GGMCAAAAACAAAGCTGTGAAAACTGAAAGCAAACTTTGAACAAAGCA AGAGACTTAAGAGTTGTATTTACAGCTTTTGTTCGATGTATGGAAATGTA CAATTTTTTTGCTACTCAAAGAAATGAGACTTAAGAGTCAACGTTAAAAG AGCCAGGAGTAAAATGTCTAGGTATGATCTCAATTGTATCGTTGTGATC AACACCAAGCTTC (SEQ ID NO: 165). The sequences of the SLtF and SLtR primers are underlined.

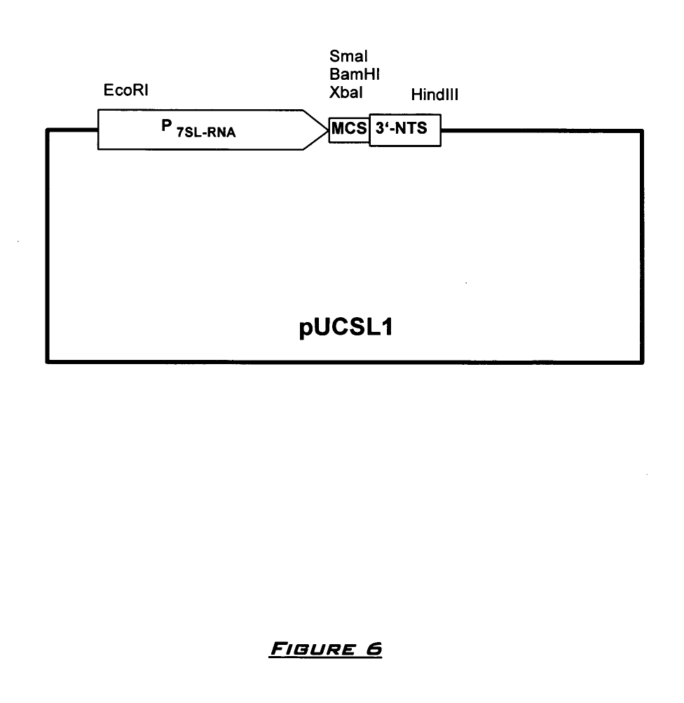

Assembly of the siRNA Delivery Cassette. The 7SL4-RNA promoter sequence was released from pCRSLp7 by digestion with Eco RI and Sma I and then inserted into a pUC19 vector at the Eco RI and Sma I cloning sites, yielding a plasmid referred to herein as pUCSLp7-1. To assemble the siRNA delivery cassette including the elements of the 7SL4-RNA promoter and the 3′-NTS fragment, the At7SL4-3′-NTS sequence was released from pCRSLt2 by digestion with Xba I and Hind III. The At7SL4-3′-NTS sequence was thereafter ligated into the Xba I and Hind III cloning sites of pUCSLp7-1 to produce a construct named pUCSL1. This construct contained the siRNA delivery cassette in a pUC19 backbone vector. The siRNA expression cassette contains the At7SL4 promoter sequence and the At7SL4-3′-NTS sequence. Between these two elements is a multiple cloning site (MCS) including sites for Sma I, Bam HI, and Xba I for insertion of target sequences (see FIG. 6).

Plant 7SL Promoter-mediated siRNA Silencing of GUS Expression in Transgenic Tobacco. A plant promoter-based system was also tested. DNA-dependent RNA polymerase III 7SL RNA genes from Arabidopsis thaliana were employed, because the transcription of these small genes is controlled exclusively by their upstream external regulatory sequence elements (USE and TATA) and terminates at a run of five to seven thymidines. These features allowed for the incorporation of these sequences into expression vectors to efficiently produce siRNA duplexes that contained three to four 3′ overhanging uridines. From an A. thaliana At7SL4, the promoter and 3′-NTS region were cloned by PCR amplification as disclosed hereinabove. The plasmid containing the At7SL4 promoter and 3′-NTS was named pUCSL1 (see FIG. 6).

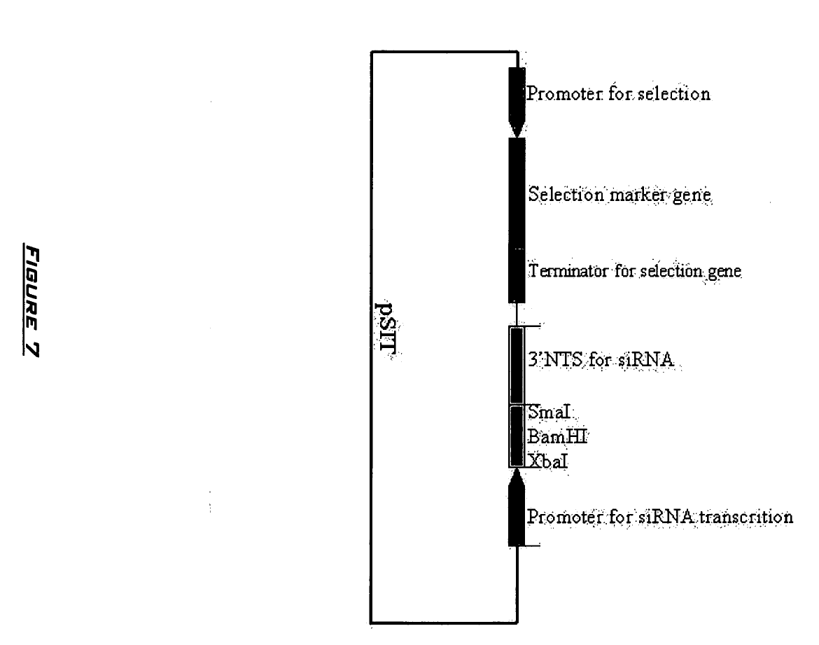

In addition to the GT1 and GT2 sequences described hereinabove, an additional 19 nt GUS mRNA sequence, referred to herein as GT3, was selected for constructing an additional siRNA template, following the general design described hereinabove. siRNA templates corresponding to GT1, GT2, and GT3 were cloned into the pSIT expression vector (see FIG. 7), which was then mobilized into A. tumefaciens C58 cells for transforming the transgenic GUS tobacco line described hereinabove (see also Hu et al., 1998). A total of 89 plants were produced containing one of these three expression constructs.

The same analysis schemes described hereinabove were employed to screen transgenic plants. It was determined that 83% of these transgenic plants exhibited a reduction in GUS enzyme activity ranging from 20 to 99%. No apparent difference in overall GUS activity reduction efficiency was observed among these three expression constructs. The observed reduction in GUS enzyme activity correlated with diminished GUS mRNA level, and with the appearance/abundance of GUS-specific siRNAs. Together, these results validated a plant promoter-based siRNA gene silencing system.

### Example 10

### pSIT System for Stable Transformation of Plants

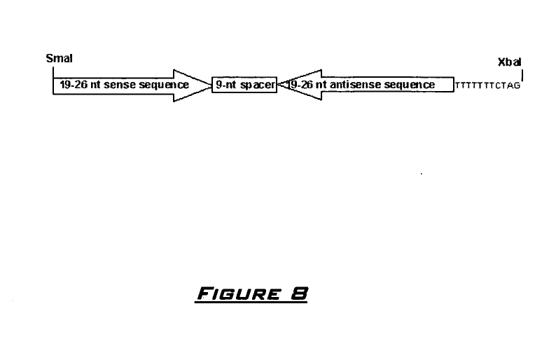

In order to introduce stably expressed miRNAs and/or siRNAs to plant tissues, a binary vector transformation system mediated by Agrobacterium was developed. The binary vector construct contained an siRNA delivery cassette and a selectable marker gene under the control of separate promoters, and is referred to herein as pSIT (small interfering RNA transformation system). See FIG. 7. Cloning sites for Sma I, Bam HI, and Xba I have been included in pSIT, and can be used for the insertion of target gene sequences in a structure designed to form a double-stranded RNA when the target gene sequences are transcribed. The insert structure is in some embodiments a 19 to 26-nucleotide sequence corresponding to the sense strand of a target gene followed by the complementary antisense sequence. The sense and antisense sequences are separated by a 9-nucleotide spacer (5′-TTCAGATGA-3′; see FIG. 8). At the 3′-end of the structure, a string of several thymidines (in some embodiments, a string of 7) was added to signal termination of transcription from the promoter.

### Example 11

### siRNA-Based Modulation of miRNA Genes

siRNA-based gene modification system can be used for modulating gene expression in plants (for example, trees). Representative, non-limiting genes the expression of which can be modulated include genes encoding the miRNAs disclosed as SEQ ID NOs: 1-59, 1247-1295, and 1662-1712 (i.e. genes comprising the nucleotide sequences disclosed as SEQ ID NOs: 60-156, 1296-1375, and 1713-1748), as well as miRNA genes involved in the regulation of the lignin and cellulose biochemical pathways. Moreover, the system is particularly useful for the manipulation of the miRNA genes that modulate multiple family members. Only a short sequence of the target gene is needed in the siRNA system, allowing the design of an siRNA target sequence to be highly specific and discernable from the other miRNA family member genes or other unknown genes which share a high sequence homology with the target member.

Based on the predicted stem-loop structure of an miRNA precursor, the nucleotide sequence of a loop region is determined. An siRNA is synthesized that hybridizes to this loop region, and an siRNA delivery cassette is generated. The siRNA delivery cassette is cloned into pSIT using the techniques described herein, and the vector is transformed into a plant cell. The transformed plant cell is used to regenerate a plant, and the expression of the plant gene targeted by the miRNA is determined in the regenerated plant and compared to the expression of the same plant gene in a wild type plant (i.e. a plant that has not been transformed with the pSIT construct.

### REFERENCES

The references listed below as well as all references cited in the specification are incorporated herein by reference to the extent that they supplement, explain, provide a background for or teach methodology, techniques and/or compositions employed herein.
Adelman et al. (1983) DNA 2:183-193.
Agrawal S (ed.) Methods in Molecular Biology, volume 20, Humana Press, Totowa, N.J., United States of America.
Altschul et al. (1990) J Mol Biol 215:403-410.
Ambros et al. (2003) Curr Biol 13:807-818.
Anterola & Lewis (2002) Phytochemistry 61:221-94.
Aravin et al. (2003) Dev Cell 5:337-350.
Ausubel et al., eds (1989) Current Protocols in Molecular Biology. Wiley, New York, N.Y., United States of America.
Bartel (2004) Cell 116:281-297.
Bartel & Bartel (2003) Plant Physiol 132:709-717.
Bevan (1984) Nucl. Acids Res 12:8711-21.
Bevan et al. (1983) Nature 304:184-187.
Binet et al. (1991) Plant Mol Biol 17:395-407.
Blochinger & Diggelmann (1984) Mol Cell Biol 4:2929-2931.
Boerjan et al. (2003) Annu Rev Plant Biol 54:519-46.
Borevitz et al. (2000) Plant Cell 12:2383-2393.
Bourouis & Jarry (1983) EMBO J. 2:1099-1104.
Callis et al. (1987) Genes Dev 1:1183-1200.
Chang et al. (1993) Plant Mol Biol Rep 11: 113-116.
Chibbar et al. (1993) Plant Cell Rep 12:506-509.
Christensen & Quail (1989) Plant Mol Biol 12:619-632.
Christou et al. (1991) Bio/Technology 9: 957-962.
Datta et al. (1990) Bio/Technology 8:736-740.
de Framond (1991) FEBS Lett 290:103-6.
Dsouza et al. (1997) Trends Genet 13:497-8.
Dostie et al. (2003) RNA 9:631-632.
Ebel et al. (1992) Biochem 31:12083-12086.
Elbashir et al. (2001a) Nature 411:494-498.
Elbashir et al. (2001b) Genes Dev 15:188-200.
Elbashir et al. (2002) Methods 26:199-213.
EP 0 292 435
EP 0332 104
EP 0 332 581
EP 0 392 225
EP 0 452 269
Firek et al. (1993) Plant Mol Biol 22:129-142.
Freier et al. (1986) Proc Natl Acad Sci USA 83:9373-9377.
Fromm (1990) Biotechnology (NY) 8:833-839.
Gallie et al. (1987) Nucl Acids Res 15:8693-8711.
Glover & Hames (1995) DNA Cloning: A Practical Approach, 2nd ed. IRL Press at Oxford University Press, Oxford; New York.
Goeddel (1990) Gene Expression Technology. Methods in Enzymology, Volume 185, Academic Press, San Diego, Calif., United States of America.
Gritz & Davies (1983) Gene 25:179-188.
Hamilton & Baulcombe (1999) Science 286:950-952.
Henikoff & Henikoff (1992) Proc Natl Acad Sci USA 89:10915-10919.
Höfgen & Willmitzer (1988) Nucl Acids Res 16:9877.
Houbaviy et al. (2003) Dev Cell 5:351-358.
Hu et al. (1998) Proc Natl Acad Sci USA 95:5407-5412.
Hudspeth & Grula (1989) Plant Molec Biol 12:579-589.
Hutvágner & Zamore (2002) Curr Opin Genet Dev 12:225-232.
Hutvágner et al. (2000) RNA 6:1445-1454.
Jefferson et al. (1987) EMBO J. 6:3901-3907.
Jin et al. (2000) EMBO J. 19:6150-6161.
Jones-Rhoades et al. (2004) Molecular Cell 14:787-799.
Karlin & Altschul (1993) Proc Natl Acad Sci USA 90:5873-5877.
Kasschau et al. (2003) Dev Cell 4:205-217.
Kawaoka & Ebinuma (2001) Phytochemistry 57:1149-1157.
Kawaoka et al. (2000) Plant J 22:289-301.
Kawasaki & Taira (2003) Nature 423:838-842.
Kliebenstein et al. (2002) Plant Physiol 130:234-243.
Koziel et al. (1993) Bio/Technology 11:194-200.
Lagos-Quintana et al. (2001) Science 294:853-858.
Lagos-Quintana et al. (2003) RNA 9:175-179.
Lagos-Quintana et al. (2002) Curr Biol 12:735-739.
Lau et al. (2001) Science 294:858-862.
Lee et al. (2002) Nature Biotechnol 20:500-505.
Lee & Ambros (2001) Science 294:862-864.
Lee et al. (1993) Cell 75:843-854.
Lee et al. (2003) Nature 425:415-419.
Lee et al. (2002) EMBO J. 21:4663-4670.
Lim et al. (2003a) Science 299:1540.
Lim et al. (2003b) Genes Dev 17:991-1008.
Liave et al. (2002). Science 297:2053-2056.
Logemann et al. (1989) Plant Cell 1:151-158.
Mayo (1987) The Theory of Plant Breeding, Second Edition, Clarendon Press, New York, N.Y., United States of America.
McBride et al., (1994) Proc Natl Acad Sci USA 91:7301-7305.
McBride & Summerfelt (1990) Plant Mol Biol 14: 269-276.
McElroy et al. (1991) Mol. Gen. Genet 231:150-160.
McElroy et al. (1990) Plant Cell 2:163-71.
Messing & Vieira (1982) Gene 19:259-268.
Michael et al. (2003) Mol. Cancer Res 1:882-891.
Mourelatos et al. (2002) Genes Dev 16:720-728.
Murashige & Skoog (1962) Physiol Plant 15:473-497.
Needleman & Wunsch (1970) J Mol Biol 48:443-453.
Negrotto et al. (2000) Plant Cell Reports 19:798-803.
Nersissian et al. (1999) Protein Sci 7:1915-1929.
Palatnik et al. (2003) Nature 425:257-263.
Park et al. (2002) Curr Biol 12:1484-1495.
Paszkowski et al. (1984) EMBO J. 3:2717-2722.
PCT International Publication No. WO 93/07278
PCT International Publication No. WO 93/21335
PCT International Publication No. WO 94/00977
Pearson & Lipman (1988) Proc Natl Acad Sci USA 85:2444-2448.
Potrykus et al. (1985) Mol Gen Genet 199:169-177.
Reinhart et al. (2002) Genes Dev 16:1616-1626.
Rhoades et al. (2002) Cell 110:513-520.
Rohrmeier & Lehle (1993) Plant Mol Biol 22:783-792.
Rothstein et al. (1987) Gene 53:153-161.
Sambrook & Russell (2001) Molecular Cloning: A Laboratory Manual, 3rd ed. Cold Spring Harbor Laboratory Press, Cold Spring Harbor, N.Y.
Scharfmann et al. (1991) Proc Natl Acad Sci USA 88:4626-4630.
Schmidhauser & Helinski (1985) J Bacteriol 164:446-455.
Schocher et al. (1986) Bio/Technology 4:1093-1096.
Shimamoto et al. (1989) Nature 338:274-276.
Silhavy (1984) Experiments with Gene Fusions. Cold Spring Harbor Laboratory, Cold Spring Harbor, N.Y., United States of America.
Singh (1986) Breeding for Resistance to Diseases and Insect Pests, Springer-Verlag, New York, N.Y., United States of America.
Skuzeski et al. (1990) Plant Mol Biol 15:65-79.
Smith & Waterman (1981) Adv Appl Math 2:482-489.
Spencer et al. (1990). Theor Appl Genet 79:625-631.
Sunkar & Zhu (2004) Plant Cell 16:2001-19.
Svab et al. (1990) Proc Natl Acad Sci USA 87:8526-8530.
Svab & Maliga (1993) Proc Natl Acad Sci USA 90:913-917.
Tamagnone et al. (1998) Plant Cell 10:135-154.
Thompson et al. (1987) EMBO J. 6:2519-2523.
Tibanyenda et al. (1984) Eur J Biochem 139:19-27.
Tijssen (1993) Laboratory Techniques in Biochemistry and Molecular Biology—Hybridization with Nucleic Acid Probes. Elsevier, New York, United States of America.
Turner et al. (1987) Cold Spring Harb Symp Quant Biol LII:123-133.
Uknes et al. (1993) Plant Cell 5:159-169.
Uknes et al. (1992) Plant Cell 4:645-656.
U.S. Pat. Nos. 4,940,935; 4,945,050; 5,036,006; 5,100,792; 5,188,642; 5,523,311; 5,591,616; and 5,614,395.
Vasil et al. (1992) Bio/Technology 10:667-674.
Vasil et al. (1993) Bio/Technology 11:1553-1558.
Wang et al. (2004) Nucleic Acids Res 32:1688-1695.
Warner et al. (1993) Plant J 3:191-201.
Weeks et al. (1993) Plant Physiol 102:1077-1084.
Welsh (1981) Fundamentals of Plant Genetics and Breeding, John Wiley & Sons, New York, N.Y., United States of America.
White et al. (1990) Nucl Acids Res 18:1062.
Wightman et al. (1993) Cell 75:855-862.
Williams et al. (1993) J Clin Invest 92:503-508.
Wood, ed. (1983) Crop Breeding, American Society of Agronomy, Madison, Wis., United States of America.
Wricke & Weber (1986) Quantitative Genetics and Selection Plant Breeding, Walter de Gruyter and Co., Berlin, Germany.
Xu et al. (1993) Plant Mol Biol 22:573-588.
Zeng & Cullen (2003) RNA 9:112-123.
Zhang et al. (1988) Plant Cell Reports 7: 379-384.
Zuker (2003) Nucleic Acids Res 31:3406-15.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for stably modulating expression of a plant gene, the method comprising:
(a) providing a vector encoding a microRNA (miRNA) targeted to the plant gene; and
(b) transforming a plant cell with the vector, whereby stable expression of the miRNA in the plant cell is provided.

2. The method of claim 1, wherein the modulating is inhibiting.

3. The method of claim 1, wherein the vector is an Agrobacterium binary vector.

4. The method of claim 1, wherein the vector comprises:
(a) a promoter operatively linked to a nucleic acid molecule encoding the miRNA molecule; and
(b) a transcription termination sequence.

5. The method of claim 4, wherein the vector is an Agrobacterium binary vector.

6. The method of claim 4, wherein the promoter is a DNA-dependent RNA polymerase III promoter.

7. The method of claim 6, wherein the promoter is selected from the group consisting of an RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, a tRNA gene promoter, and functional derivatives thereof.

8. The method of claim 7, wherein the Arabidopsis thaliana 7SL RNA gene promoter comprises the sequence presented in SEQ ID NO: 162.

9. The method of claim 4, wherein the nucleic acid sequence encoding the microRNA (miRNA) molecule comprises a sense region, an antisense region, and a loop region, positioned in relation to each other such that upon transcription, a resulting RNA transcript is capable of forming a hairpin structure via intramolecular hybridization of the sense strand and the antisense strand.

10. The method of claim 9, wherein the nucleic acid sequence encoding the microRNA (miRNA) molecule comprises a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

11. The method of claim 1, wherein the plant gene comprises a nucleotide sequence selected from the group consisting of SEQ ID NOs: 176-781, 1376-1553, and 1749-1837, and sequences at least 80% identical to any of SEQ ID NOs: 176-781, 1376-1553, and 1749-1837.

12. The method of claim 1, wherein the plant is a dicot.

13. The method of claim 1, wherein the plant is a monocot.

14. The method of claim 1, wherein the plant is a tree.

15. The method of claim 14, wherein the tree is an angiosperm.

16. The method of claim 14, wherein the tree is a gymnosperm.

17. The method of claim 14, wherein the tree is a member of the genus Populus.

18. The method of claim 1, wherein the stable expression of the microRNA (miRNA) in the plant occurs in a location or tissue selected from the group consisting of epidermis, root, vascular tissue, xylem, meristem, cambium, cortex, pith, leaf, flower, seed, and combinations thereof.

19. A method for stably modulating expression of a plant gene, the method comprising:
(a) transforming a plurality of plant cells with an Agrobacterium tumefaciens binary vector comprising:
(i) a nucleic acid sequence encoding a selectable marker; and
(ii) a nucleic acid sequence encoding a microRNA (miRNA) operatively linked to a promoter and a transcription termination sequence;
(b) treating the plant cells with a drug under conditions sufficient to kill those plant cells that did not receive the binary vector, wherein the selectable marker provides resistance to the drug, to create a first plurality of transformed plant cells;
(c) growing the first plurality of transformed plant cells under conditions sufficient to select for a second plurality of transformed plant cells that have integrated the binary vector into their genomes;
(d) screening the second plurality of transformed plant cells for expression of the miRNA encoded by the expression vector;
(e) selecting a transformed plant cell that expresses the miRNA; and
(f) regenerating the plant from the transformed plant cell that expresses the miRNA, whereby expression of the gene in the plant is stably modulated.

20. A vector for stably expressing a microRNA (miRNA) molecule in a plant, the vector comprising:
(a) a promoter operatively linked to a nucleic acid molecule encoding the miRNA molecule; and
(b) a transcription termination sequence.

21. The vector of claim 20, wherein the vector is an Agrobacterium binary vector.

22. The vector of claim 20, wherein the promoter is a DNA-dependent RNA polymerase III promoter.

23. The vector of claim 22, wherein the promoter is selected from the group consisting of RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, a tRNA gene promoter, and functional derivatives thereof.

24. The vector of claim 23, wherein the Arabidopsis thaliana SL7 RNA gene promoter comprises the sequence presented in SEQ ID NO: 162.

25. The vector of claim 20, wherein the nucleic acid sequence encoding the microRNA (miRNA) molecule comprises a sense region, an antisense region, and a loop region, positioned in relation to each other such that upon transcription, a resulting RNA transcript is capable of forming a hairpin structure via intramolecular hybridization of the sense strand and the antisense strand.

26. The vector of claim 25, wherein the nucleic acid sequence encoding the microRNA (miRNA) molecule comprises a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

27. The vector of claim 20, wherein the plant gene has a nucleotide sequence comprising a nucleotide sequence selected from the group consisting of any of SEQ ID NOs: 176-781, 1376-1553, and 1749-1837, and nucleotide sequences at least 80% identical to any of SEQ ID NOs: 176-781, 1376-1553, and 1749-1837.

28. A kit comprising the vector of claim 20 and at least one reagent for introducing a vector of claim 18 into a plant cell.

29. The kit of claim 28, further comprising instructions for introducing the vector into a plant cell.

30. A plant cell comprising a vector of claim 20.

31. A transgenic plant comprising a vector of claim 20.

32. Transgenic seed or progeny from a transgenic plant of claim 31.

33. A method for stably inhibiting the expression of a gene in a plant cell, the method comprising stably transforming the plant cell with a vector encoding a microRNA (miRNA) molecule, wherein the miRNA molecule comprises a nucleotide sequence at least 70% identical to a contiguous 17-24 nucleotide subsequence of the gene.

34. The method of claim 33, wherein the gene is selected from the group consisting of coniferaldehyde-5-hydroxylase (Cald5H), a lignin-related gene, a cellulose-related gene, a hemicellulose-related gene, a hormone-related gene, a disease-related gene, a stress-related gene, a growth-related gene, and a transcription factor gene.

35. The method of claim 34, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCoAOMT), caffeate O-methyltransferase (COMT), ferulate-5-hydroxylase (F5H), cinnamate-4-hydroxylase (C4H), p-coumarate-3-hydroxylase (C3H), and phenylalanine ammonia lyase (PAL).

36. The method of claim 34, wherein the cellulose-related gene is selected from the group consisting of cellulose synthase, cellulose synthase-like, glucosidase, glucan synthase, and sucrose synthase.

37. The method of claim 34, wherein the hormone-related gene is selected from the group consisting of isopentyl transferase (ipt), gibberellic acid (GA) oxidase, auxin (AUX), and a rooting locus (ROL) gene.

38. The method of claim 33, wherein the miRNA molecule is encoded by a nucleic acid molecule comprising a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

39. The method of claim 33, wherein the plant gene comprises a nucleotide sequence selected from the group consisting of any of SEQ ID NOs: 176-781, 1376-1553, and 1749-1837, and nucleotide sequences at least 80% identical to any of SEQ ID NOs: 176-781, 1376-1553, and 1749-1837.

40. A method for enhancing the expression of a gene in a plant cell, the method comprising introducing into the plant cell a vector encoding a short interfering RNA (siRNA) molecule comprising a sequence that hybridizes under physiological conditions to a loop region or a stem region of a pre-microRNA that comprises a microRNA (miRNA) that modulates expression of the gene, thereby resulting in downregulation of expression of the miRNA and enhanced expression of the gene.

41. The method of claim 40, wherein the microRNA (miRNA) comprises a nucleotide sequence selected from the group consisting of any of SEQ ID NOs: 1-59, 1247-1295, and 1662-1712, and nucleotide sequences at least 70% identical to any of SEQ ID NOs: 1-59, 1247-1295, and 1662-1712.

42. An expression vector comprising a nucleic acid sequence encoding a microRNA (miRNA) molecule that stably down regulates expression of a plant gene.

43. The expression vector of claim 42, wherein the nucleic acid sequence encoding the microRNA (miRNA) molecule comprises a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

44. The expression vector of claim 42, wherein the miRNA comprises a nucleotide sequence of about 17-24 contiguous nucleotides with up to 5 mismatches of a ribonucleic acid (RNA) transcribed from a gene selected from the group consisting of a lignin-related gene, a cellulose-related gene, a hemicellulose-related gene, a hormone-related gene, a disease-related gene, a stress-related gene, a medicine-related gene, and a transcription factor gene.

45. The expression vector of claim 44, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCoAOMT), caffeate O-methyltransferase (COMT), ferulate-5-hydroxylase (F5H), cinnamate-4-hydroxylase (C4H), p-coumarate-3-hydroxylase (C3H), and phenylalanine ammonia lyase (PAL).

46. The expression vector of claim 44, wherein the cellulose-related gene is selected from the group consisting of cellulose synthase, cellulose synthase-like, glucosidase, glucan synthase, and sucrose synthase.

47. The expression vector of claim 44, wherein the hormone-related gene is selected from the group consisting of isopentyl transferase (ipt), gibberellic acid (GA) oxidase, auxin (AUX), and a rooting locus (ROL) gene.

48. A plant cell comprising an expression vector of claim 42.

49. The plant cell of claim 48, wherein the plant cell is from a plant selected from the group consisting of poplar, pine, eucalyptus, sweetgum, other tree species, tobacco, Arabidopsis, rice, corn, wheat, cotton, potato, and cucumber.

50. A vector for the stable expression of a microRNA (miRNA) in a plant, wherein the vector comprises a promoter for expressing the miRNA, a transcription termination sequence, and a cloning site between the promoter and the transcription termination sequence into which a nucleic acid molecule encoding the miRNA can be cloned.

51. The vector of claim 50, wherein the microRNA (miRNA) comprises a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

52. The vector of claim 51, wherein the promoter is a DNA-dependent RNA polymerase III promoter.

53. The vector of claim 52, wherein the promoter is selected from the group consisting of RNA polymerase Ill H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof.

54. The vector of claim 53, wherein the Arabidopsis thaliana 7SL RNA gene promoter comprises SEQ ID NO: 162.

55. The vector of claim 51, wherein the vector is a plasmid vector.

56. The vector of claim 55, wherein the vector further comprises a selectable marker.

57. The vector of claim 55, wherein the cloning site comprises a recognition sequence for at least one restriction enzyme that is not present elsewhere in the plasmid vector.

58. A method for stably modulating expression of a plant gene, the method comprising:
(a) transforming a plurality of plant cells with a vector comprising a nucleic acid sequence encoding a microRNA (miRNA) operatively linked to a promoter and a transcription termination sequence;
(b) growing the plant cells under conditions sufficient to select for a plurality of transformed plant cells that have integrated the vector into their genomes;
(c) screening the plurality of transformed plant cells for expression of the miRNA encoded by the vector;
(d) selecting a transformed plant cell that expresses the miRNA; and
(e) regenerating the plant from the transformed plant cell that expresses the miRNA, whereby expression of the plant gene is stably modulated.

59. The method of claim 58, wherein the nucleic acid sequence encoding the microRNA (miRNA) comprises:
(a) a sense region;
(b) an antisense region; and
(c) a loop region,
wherein the sense, antisense, and loop regions are positioned in relation to each other such that upon transcription, a resulting RNA transcript is capable of forming a hairpin structure via intramolecular hybridization of the sense strand and the antisense strand.

60. The method of claim 58, wherein the vector is an Agrobacterium binary vector that comprises a nucleic acid encoding a selectable marker operatively linked to a promoter.

61. The method of claim 58, wherein the nucleic acid sequence encoding the miRNA comprises a nucleotide sequence selected from the group consisting of any of SEQ ID NOs: 1-59, 1247-1295, and 1662-1712, and sequences at least 70% identical to any of SEQ ID NOs: 1-59, 1247-1295, and 1662-1712.

62. The method of claim 58, wherein the plant gene comprises a nucleotide sequence selected from the group consisting of any of SEQ ID NOs: 60-156, 1296-1375, and 1713-1748, and nucleotide sequences at least 80% identical to any of SEQ ID NOs: 60-156, 1296-1375, and 1713-1748.

63. An isolated microRNA (miRNA) comprising a nucleotide sequence of one of SEQ ID NOs: 1-59, 1247-1295, and 1662-1712, and sequences at least 70% identical to any of SEQ ID NOs: 1-59, 1247-1295, and 1662-1712.

64. The isolated microRNA (miRNA) of claim 63, wherein the miRNA modulates expression of a gene expressed in a tree of the genus Populus.

65. The isolated microRNA (miRNA) of claim 64, wherein the tree is a Populus trichocarpa tree.

66. The isolated microRNA (miRNA) of claim 63, wherein the miRNA modulates expression of a gene expressed in a tree of the genus Pinus.

67. The isolated microRNA (miRNA) of claim 66, wherein the tree is a Pinus taeda tree.
